(19) United States
(12) Patent Application Publication
(10) Pub. No.: US 2023/0174930 A1
(43) Pub. Date: Jun. 8, 2023

(54) Organotypic Brain Slice Culture Model of Human Neuroinflammatory Environment Associated with Neurodegeneration

(71) Applicant: Tiaki Therapeutics Inc., Cambridge, MA (US)

(72) Inventors: Lauren H. Martens, Wayland, MA (US); Kelley C. Larson, Quincy, MA (US); Barbara A. Tate, Bedford, MA (US)

(21) Appl. No.: 17/964,715

(22) Filed: Oct. 12, 2022

Related U.S. Application Data
(60) Provisional application No. 63/254,835, filed on Oct. 12, 2021.

(51) Int. Cl. C12N 5/079

(52) U.S. Cl. CPC C12N 5/0618; C12N 2503/04

(57) ABSTRACT

Described herein are methods, compositions, and assay systems that provide a useful ex vivo model for neurodegenerative disorders, by providing a whole brain organotypic slice culture model maintains viability of mature CNS cells from adult animals. In addition, the whole brain slice cultures described herein exhibit hallmarks of chronic neuroinflammation, where hallmarks are consistent across age-related neurodegenerative disorders. Also provided herein is a cultured whole brain slice which exhibits at least one characteristic of chronic neuroinflammation.

### CROSS REFERENCE TO RELATED APPLICATIONS

This application claims the benefit of and priority to U.S. Provisional Application No. 63/254,835, filed Oct. 12, 2021, which is hereby incorporated by reference in its entirety.

Organotypic brain slice culture model of human neuroinflammatory environment associated with neurodegeneration.

### BACKGROUND

Neurodegenerative disorders, such as Alzheimer's disease (AD), Huntington's disease (HD), and amyotrophic lateral sclerosis (ALS), are age-related diseases resulting in neuronal dysfunction, neuronal death, cognitive decline, and death. With an aging population, prevalence of these disorders will continue to increase, creating a significant public health issue. Despite decades of research, there are no cures or preventative treatments for these disorders.

Part of the challenge of therapeutic discovery for these diseases lays in the lack of suitable model systems that recapitulate the molecular aspects of these diseases. While there are known genetic predispositions for certain neurodegenerative disorders, many patients with AD and ALS have no known genetic predispositions, i.e., their cases are idiopathic. While the genetic basis for HD is known to stem from mutations in the huntingtin gene, the molecular mechanisms by which mutant huntingtin contributes to neurodegeneration are unclear.

Organotypic brain slice cultures represent an advance in the field of therapeutic discovery for neurodegenerative diseases. Organotypic brain slices cultured under conditions in which the slice retains the three-dimensional tissue organization, morphology, composition and physiological properties of the source organ, presenting an advantage over traditional primary cell culture models that typically comprise cultures of cells that have been dissociated from the originating tissue.

Organotypic brain slice culture models have been established for Alzheimer's disease, Huntingtin's disease, and ALS. See, Croft et al., Preparation of organotypic brain slice cultures for the study of Alzheimer's disease, F1000Research 7:592 (2018). See also, Sheppard et al, Lipopolysaccharide-induced neuroinflammation induces presynaptic disruption through a direct action on brain tissue involving microglia-derived interleukin 1 beta, Journal of Neuroinflammation 16:106 (2019). See also, Andre et al., A novel ex vivo Huntington's disease model for studying GABAergic neurons and cell grafts by laser microdissection, PLoSONE 13(3) (2018). However, these models are limited in that they largely rely on immature CNS tissue from embryonic or early postnatal animals, which are profoundly different from mature adult brain tissue that more accurately reflects age-related changes characteristic of the disease state.

Organotypic hippocampal slice culture models have been established from brain tissue of older animals (e.g., 1 year-old mice). See Schommer et al., Method for organotypic tissue culture in the aged animal, MethodsX 4 (2017) 166-171, which is hereby incorporated by reference in its entirety. However, hippocampal slice culture involves dissection of the hippocampus from the harvested brain for culturing. Hippocampal tissue is distinguished from other regions of the brain in that it comprises neural stem cell and progenitor cell populations with the potential for neurogenesis. By contrast, neurons from other regions of the brain, e.g., cortical regions, largely lack the potential for neurogenesis.

Other studies have explored methods for the organotypic culture of whole brain slices from adult rodents. See Staal, Jerome A et al. “Characterization of cortical neuronal and glial alterations during culture of organotypic whole brain slices from neonatal and mature mice.” PloS one vol. 6,7 (2011): e22040. doi:10.1371/journal.pone.0022040, which is hereby incorporated by reference in its entirety. See also Mewes, Agneta et al. “Organotypic brain slice cultures of adult transgenic P301S mice—a model for tauopathy studies.” PloS one vol. 7,9 (2012): e45017. doi:10.1371/journal.pone.0045017, which is hereby incorporated by reference in its entirety. However, the cultures described suffer from major limitations, such as a significant loss of neuronal viability in long-term culture (e.g., 4 DIV), or loss of major cell types, e.g., mature microglia, in the cultured slices.

Accordingly, there remains a lack of brain slice culture models featuring mature CNS cells in their native architecture/milieu, which may be useful in modeling aspects of CNS neurodegenerative diseases such as AD, HD, and ALS.

### SUMMARY OF THE INVENTION

Described herein is a cultured whole brain slice obtained from an adult rodent of at least 17 days of age, wherein the brain slice comprises adult microglia and has been cultured for at least 3 days in vitro. In certain aspects, described herein is a cultured whole brain slice obtained from an adult rodent, wherein the cultured whole brain slice has been cultured in a serum-free or reduced serum culture medium, and wherein the cultured whole brain slice has maintained viability in the serum-free or reduced serum culture medium for one day or more. In certain embodiments, prior to culturing in the serum-free or reduced serum culture medium, the cultured whole brain slice was cultured in a recovery cell culture medium. In certain embodiments, the recovery cell culture medium comprises serum, optionally more than about 5% serum, optionally at least about 10% serum, optionally about 20% serum, optionally wherein the serum is bovine, equine, ovine, caprine or porcine, optionally wherein the serum is heat-inactivated. In certain embodiments, the cultured whole brain slice was cultured in the recovery cell culture medium for more than 2 days, optionally for at least 3 days, optionally for at least 4 days. In certain embodiments, the serum-free or reduced serum culture medium comprises no more than about 5% serum, optionally wherein the serum-free or reduced serum culture medium is a serum-free culture medium. In certain embodiments, the serum-free or reduced serum culture medium comprises no more than half the serum concentration present in the recovery cell culture medium.

In certain aspects, described herein is a cultured whole brain slice, wherein the cultured whole brain slice exhibits at least one characteristic of chronic neuroinflammation. In certain embodiments, at least one characteristic of chronic inflammation comprises increased release of one or more pro-inflammatory cytokines as compared to naïve rodent cerebrospinal fluid, optionally wherein the cultured whole brain slice exhibits the increased release at DIV1, optionally wherein the cultured whole brain slice exhibits the increased release at DIV5, DIV6, DIV7, or greater than DIV7, optionally wherein the one or more pro-inflammatory cytokines are selected from IL-6, TNF-α, IL-1, and INFγ. In certain embodiments, the at least one characteristic of chronic inflammation comprises a gene expression signature characteristic of chronic neuroinflammation. In certain embodiments, the gene expression signature comprises increases in one or more genes selected from the group consisting of: C3, Gfap, Cd9, Msn, Cd68, Klk6, Gjb1, Trem2, Gusb, Trf, Bcas1, Tgfb1, Ccnd1, Grn, Olig2, Tnfrsf1a, Fgf2, Sgpl1, C1qc, Mbp, S100b, C1qa, Myrf, Csf1, Cxcl16, Cd14, Nes, Gsn, Lrrc25, Mag, Ugt8a, Car2, Tspo, Sox10, Cybb, Spi1, Stat3, Pmp22, Serpinb6a, and Arhgef10, and, optionally, the gene expression signature comprises decreases in one or more genes selected from the group consisting of: Gabra1, Syt1, Gabrb2, Slc12a5, Pcsk2, Calb1, Grin1, Rasgrp1, Scn1a, Grin2b, Cx3cl1, Drd1, Gabrg2, Scn2a1, Vip, Gad1, Camk4, Prkcg, Syt13, Thy1, Cntnap1, Cnksr2, Ryr2, Cnr1, Sncb, Unc13a, Nell2, Cdk5r1, Cacna1b, Epha7, Ptprn2, Grin2a, Gabra4, Cds1, Fgf14, Grm1, Ipcef1, Htr1a, Slc8a1, Gad2.

In certain aspects, described herein is a cultured whole brain slice, wherein the cultured whole brain slice exhibits a gene expression signature, wherein the gene expression signature comprises stable correlation with a human neuroinflammatory disease signature of interest. In certain embodiments, the human neuroinflammatory disease signature of interest is selected from the group consisting of AD neuroinflammatory subset, Huntington's disease (HD), ALS neuroinflammatory subset, progressive supranuclear palsy (PSP), and frontotemporal dementia (FTD). In certain embodiments, the cultured whole brain slice is obtained from a rodent. In certain embodiments, the rodent is at least 17 days of age. In certain embodiments, the adult rodent is at least 19 days of age. In certain embodiments, the adult rodent is at least 21 days of age, optionally at least 42 days of age. In certain embodiments, the rodent is a mouse or rat. In certain embodiments, the cultured whole brain slice comprises cortical brain tissue. In certain embodiments, the cultured whole brain slice further comprises subcortical brain tissue. In certain embodiments, the cortical and subcortical brain tissue comprises neurons or their progenitor cells. In certain embodiments, the cortical and subcortical brain tissue comprises microglia or their progenitor cells. In certain embodiments, the cortical and subcortical brain tissue comprises astrocytes. In certain embodiments, the cortical and subcortical brain tissue comprises oligodendrocytes or their progenitor cells. In certain embodiments, the cortical and subcortical brain tissue comprises endothelial cells. In certain embodiments, the cultured whole brain slice has a thickness of about 100-400 μm, optionally about 300 μm.

In certain aspects, described herein is an assay system, comprising the cultured whole brain slice of any one of the preceding claims and a cell culture medium. In certain embodiments, the cell culture medium is a serum-free or reduced serum culture medium. In certain embodiments, the assay system comprises a plurality of cultured whole brain slices according to any one of the preceding claims. In certain embodiments, the assay system further comprises a candidate therapeutic. In certain embodiments, the candidate therapeutic is administered to the cultured whole brain slice.

In certain aspects, described herein is a method of producing a cultured whole brain slice, comprising: culturing a whole brain slice obtained from a rodent in a recovery cell culture medium comprising serum; and culturing the whole brain slice in a serum-free or reduced serum culture medium. In certain embodiments, the recovery cell culture medium comprises more than about 5% serum, optionally at least about 10% serum, optionally about 20% serum, optionally wherein the serum is bovine, equine, ovine, caprine or porcine, optionally wherein the serum is heat-inactivated. In certain embodiments, the method comprises culturing the cultured whole brain slice in the recovery cell culture medium for more than 2 days, optionally for at least 3 days, optionally for at least 4 days. In certain embodiments, the method comprises culturing the brain slice in the serum-free or reduced serum culture medium for at least 1 day, optionally for at least 2, 3, 4, 5, or more than 5 days. In certain embodiments, the serum-free or reduced serum culture medium comprises no more than about 5% serum, optionally wherein the serum-free or reduced serum culture medium is a serum-free culture medium. In certain embodiments, the serum-free or reduced serum culture medium comprises no more than half the serum concentration present in the recovery cell culture medium. In certain embodiments, the whole brain slice is obtained from an adult rodent at least 17 days of age, optionally at least 19 days of age, optionally at least 21 days of age, optionally at least 42 days of age. In certain embodiments, the whole brain slice comprises cortical and subcortical brain tissue. In certain embodiments, cortical and subcortical brain tissue comprises neurons or their progenitors. In certain embodiments, the cortical and subcortical brain tissue comprises microglia or their progenitors. In certain embodiments, the cortical and subcortical brain tissue comprises astrocytes. In certain embodiments, the cortical and subcortical brain tissue comprises oligodendrocytes or their precursors.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 are images of the brain slice overview (middle) and brain slices in culture (lower).

FIG. 2 is an illustration depicting process of culture conditions of brain slices followed by tissue and media analysis.

FIG. 3A is a graph depicting elevated Interleukin-6 (IL-6) in brain slice cultures during the acute injury and chronic neuroinflammation phases.

FIG. 3B is a graph depicting elevated Tumor Necrosis Factor Alpha (TNFa) in brain slice cultures during the acute injury and chronic neuroinflammation phases.

FIG. 4 are graphs showing differential gene expression in organotypic cortical slices (OGCS) over time.

FIG. 5 is a heatmap of OGCS gene expression over days in culture (results are average of n=2-3 per time point).

FIG. 6 is a graph showing comparisons of gene expression changes of Alzheimer's Disease (AD) transcriptome and gene expression changes of OGCS after 6 days of culture.

FIG. 7 are graphs showing comparisons of gene expression changes of Alzheimer's Disease (AD) transcriptome and gene expression changes of OGCS after the indicated number of days of culture (DIV1, 3, 4, 6, 8, 11 and 14).

FIG. 8 are graphs showing comparisons of gene expression changes of ALS glial activation transcriptomes of sub-populations from the indicated brain regions and of gene expression changes of OGCS after 8 days of culture.

FIG. 9 are heatmaps depicting the OGCS gene expression patterns and the ALS glial activation sub-population gene expression patterns with respect to inflammation, synaptic function and neurodegenerative genes. ‘mcl” is motor cortex lateral; “mcm” is medial motor cortex, “fc” is frontal cortex.

FIG. 10 are graphs showing comparisons of gene expression changes of ALS oxidative stress sub-populations from the indicated brain regions and of gene expression changes of OGCS after 8 days of culture.

FIG. 11 are heatmaps depicting the OGCS gene expression patterns and the ALS oxidative stress sub-population gene expression patterns with respect to inflammation, synaptic function and neurodegenerative genes. ‘mcl” is motor cortex lateral; “mcm” is medial motor cortex, “fc” is frontal cortex.

FIG. 12 are graphs showing comparisons of gene expression changes of ALS retrotransposon sub-populations from the indicated brain regions and of gene expression changes of OGCS after 8 days of culture.

FIG. 13 are heatmaps depicting the OGCS gene expression patterns and the ALS retrotransposon/TDP-43 sub-population gene expression patterns with respect to inflammation, synaptic function and neurodegenerative genes. ‘mcl” is motor cortex lateral; “mcm” is medial motor cortex, “fc” is frontal cortex.

FIG. 14 are graphs showing alignment of gene expression of Huntington's disease datasets GSE26927 and GSE3790 (caudate nucleus/CN) and gene expression of OGCS.

FIG. 15 are graphs showing alignment of gene expression samples from Huntington's disease patients from caudate nucleus or frontal cortex (Brodmann Area 9/BA9) and gene expression of OGCS.

FIG. 16 are graphs showing alignment of gene expression of Huntington's disease patients from caudate nucleus or Brodmann Area 9 and gene expression of OGCS.

FIG. 17 is a heatmap showing gene expression signatures of AD, HD and ALS neuroinflammatory diseases and OGCS for the indicated pathways.

FIG. 18 is a heatmap showing gene expression signatures for the indicated pathways of AD, HD and ALS neuroinflammatory diseases, OGCS, and OGCS (DIV5-8) treated with recombinant msTGFβ.

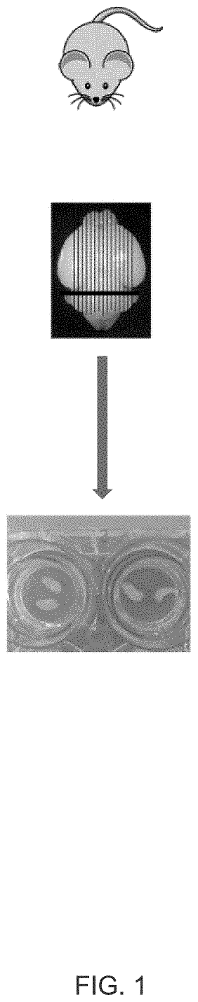

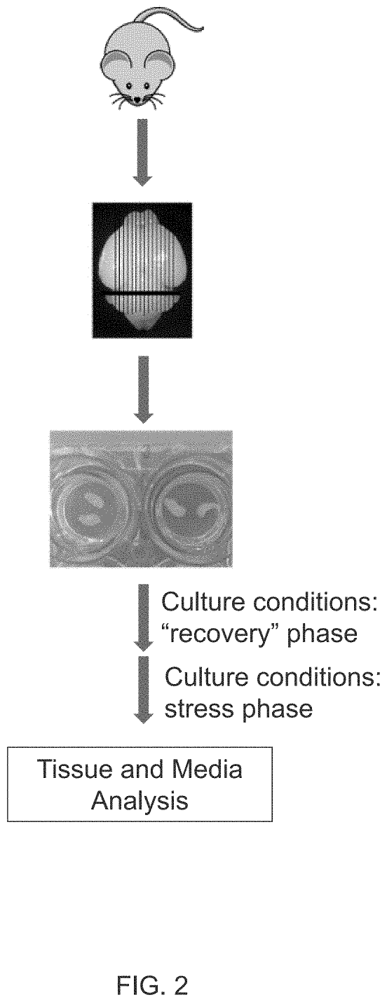

### DETAILED DESCRIPTION OF THE INVENTION

### Definitions

Terms used in the claims and specification are defined as set forth below unless otherwise specified.

The term, “reduced serum culture” as used herein means less than 20% (e.g., less than 15%, 10%, 5%, 2%, 1% or serum free) serum of the total volume of culture medium. “Serum free” refers to less than about 1% serum (e.g., 0.3% serum).

The term, “about”, as used herein, refers to +/−15% of the specified value.

Abbreviations used in this application include the following: DIV, which refers to days of culture ex vivo, OGCS refers to organotypic cortical slices. AD refers to Alzheimer's disease, HD refers to Huntington's disease, ALS refers to amyotrophic lateral sclerosis, PSP refers to progressive supranuclear palsy, and FTD refers to frontotemporal dementia.

It must be noted that, as used in the specification and the appended claims, the singular forms “a,” “an” and “the” include plural referents unless the context clearly dictates otherwise.

Overview

The methods, compositions, and systems described herein provide a useful ex vivo model for neurodegenerative disorders, by providing a whole brain organotypic slice culture model maintains viability of mature CNS cells from adult animals, e.g., over 4 DIV. In addition, the whole brain slice cultures exhibit hallmarks of chronic neuroinflammation, where hallmarks are consistent across age-related neurodegenerative disorders such as, e.g., AD, HD, and ALS.

Accordingly, provided herein is a cultured whole brain slice from an adult animal, wherein the cultured whole brain slice has been cultured for at least 3 DIV. Also provided herein is a cultured whole brain slice which exhibits at least one characteristic of chronic neuroinflammation.

In some embodiments, the cultured whole brain slice has maintained viability of adult microglia. In some embodiments, the cultured whole brain slice has maintained viability of adult microglia for at least 3 DIV.

Animal Sources of Slice

In some embodiments, the adult animal from which the brain slice is obtained is a mammal. In some embodiments, the mammal is a rodent.

In some embodiments, the rodent is a mouse. A skilled artisan would readily recognize that the whole brain slice may be obtained from any suitable mouse. In some embodiments, the mouse is of the species Mus musculus. In some embodiments, the mouse is a laboratory mouse. The laboratory mouse strain may be of an inbred mouse strain. The laboratory mouse may be of an outbred mouse strain. In some embodiments, the mouse is a wild-type mouse, e.g., does not comprise an engineered genetic manipulation, e.g., is not a transgenic mouse, or knockout or knockin mouse, or a genome-edited mouse. In some embodiments, the mouse comprises a spontaneous mutation.

In some embodiments, the mouse is a genetically engineered mouse, comprising an engineered genetic manipulation that increases expression, decreases expression or removes expression of a gene or genes of interest

Exemplary mouse strains from which the brain slice may be obtained include, but are not limited to, C57BL, C57BL/6, 129Sv, BALB/C, ICR, NOD, SJL, Swiss-Webster, C3H, DBA, FVB, CBA, Hairless, MRL 1pr, NZB, NCR, AKR, A CFTR, CD1, SCID, nude, NMRI, JPNL3, and SENCAR mice, and any substrains thereof.

In some embodiments, the rodent is a rat. The rat may be of the species Rattus norvegicus. A skilled artisan would readily recognize that the whole brain slice may be obtained from any suitable rat. In some embodiments, the rat is a laboratory rat. The laboratory rat strain may be of an inbred rat strain. The laboratory rat may be of an outbred rat strain. In some embodiments, the rat is a wild-type rat, e.g., does not comprise an engineered genetic manipulation, e.g., is not a transgenic rat, or knockout or knockin rat, or a genome-edited rat. In some embodiments, the rat comprises a spontaneous mutation.

In some embodiments, the rat is a genetically engineered rat, comprising an engineered genetic manipulation.

Exemplary rat strains from which the brain slice may be obtained include, but are not limited to, Wistar, Long-Evans, Sprague Dawley, BBDP, Brattleboro, hairless, Lewis, RCS, shaking rat Kawasaki, and Zucker.

In some embodiments, the age of the rodent from which the brain slice is harvested is at least 17 days (postnatal day 17). In some embodiments, the age of the rodent from which the brain slice is harvested is at least 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 90, 120, 150, 180, 270, 365 days, or more than 365 days. In particular embodiments, the age of the rodent from which the brain slice is harvested is at least 19 days (postnatal day 19). In particular embodiments, the age of the rodent from which the brain slice is harvested is at least 21 days (postnatal day 21). In particular embodiments, the age of the rodent from which the brain slice is harvested is at least 33 days (postnatal day 33). In particular embodiments, the age of the rodent from which the brain slice is harvested is at least 42 days (postnatal day 42). In particular embodiments, the age of the rodent from which the brain slice is harvested is at least 77 days (postnatal day 77). In some embodiments, the brain slice has a thickness of between about 50-1000 μm. In some embodiments, the brain slice has a thickness of between about 100-500 μm. In some embodiments, the brain slice has a thickness of between about 150-450 μm. In some embodiments, the brain slice has a thickness of between about 200-400 μm. In particular embodiments, the brain slice has a thickness of about 300 μm.

In some embodiments, the brain slice comprises forebrain and midbrain regions. In some embodiments, the brain slice comprises the hindbrain region. In some embodiments, the brain slice does not comprise the hindbrain region.

Methods of Preparing

Described herein are methods of preparing assay systems that are a useful ex vivo model for neurodegenerative disorders, by providing a whole brain organotypic slice culture model maintains viability of mature CNS cells from adult animals. Thus, described herein are methods of producing a cultured whole brain slice, comprising culturing a whole brain slice obtained from a rodent in a recovery cell culture medium comprising serum; and culturing the whole brain slice in a serum-free or reduced serum culture medium. In certain embodiments, the methods comprise producing the recovery cell culture medium comprises more than about 5% serum, optionally at least about 10% serum, optionally about 20% serum, optionally wherein the serum is bovine, equine, ovine, caprine or porcine, optionally wherein the serum is heat-inactivated.

In certain embodiments, the methods comprise culturing the cultured whole brain slice in the recovery cell culture medium for more than 2 days, optionally for at least 3 days, optionally for at least 4 days. In certain embodiments, the methods comprise culturing the brain slice in the serum-free or reduced serum culture medium for at least 1 day, optionally for at least 2, 3, 4, 5, or more than 5 days. In certain embodiments, the serum-free or reduced serum culture medium comprises no more than about 5% serum, optionally wherein the serum-free or reduced serum culture medium is a serum-free culture medium. In certain embodiments, the reduced serum culture medium comprises 0-5% serum, 1-6% serum, 1-10% serum, 0-2% serum, or 5-10% serum. In certain embodiments, the serum-free or reduced serum culture medium comprises no more than half the serum concentration present in the recovery cell culture medium. In certain embodiments, the whole brain slice is obtained from an adult rodent at least 17 days of age, optionally at least 19 days of age, optionally at least 21 days of age, optionally at least 42 days of age. In certain embodiments, the whole brain slice comprises cortical brain tissue. In certain embodiments, the whole brain slice further comprises sub-cortical brain tissue. In certain embodiments, the cortical and subcortical brain tissue comprises neurons or their progenitors. In certain embodiments, the cortical and subcortical brain tissue comprises microglia or their progenitors. In certain embodiments, the cortical and subcortical brain tissue comprises astrocytes. In certain embodiments, the cortical and subcortical brain tissue comprises oligodendrocytes or their precursors.

Methods of Use

The methods, compositions, and assay systems described herein provide a useful ex vivo model for neurodegenerative disorders, by providing a whole brain organotypic slice culture model maintains viability of mature CNS cells from adult animals. Thus, in certain embodiments the methods, systems and compositions described herein are useful for the understanding and identification of pathways and genes that are useful for development of treatments for neurodegenerative disorders, such as, but not limited to, Alzheimer's disease (AD), Huntington's disease (HD), amyotrophic lateral sclerosis (ALS), progressive supranuclear palsy (PSP), and frontotemporal dementia (FTD).

In certain embodiments, the methods, systems, and compositions described herein are useful for identification of gene expression signatures that are correlated with human neuroinflammatory disease signatures including, but not limited to, Alzheimer's disease (AD), Huntington's disease (HD), amyotrophic lateral sclerosis (ALS), progressive supranuclear palsy (PSP), and frontotemporal dementia (FTD).

In certain embodiments, the methods, systems, and compositions described herein are useful for identification of a drug target for the treatment of human neuroinflammatory disease including, but not limited to, Alzheimer's disease (AD), Huntington's disease (HD), amyotrophic lateral sclerosis (ALS), progressive supranuclear palsy (PSP), and frontotemporal dementia (FTD).

In certain embodiments, the methods, systems, and compositions described herein are useful for identification and/or development of a candidate therapeutic for the treatment or prevention of a neuroinflammatory disease including, but not limited to, Alzheimer's disease (AD), Huntington's disease (HdD), amyotrophic lateral sclerosis (ALS), progressive supranuclear palsy (PSP), and frontotemporal dementia (FTD).

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry^{3rd} Ed. (Plenum Press) Vols A and B (1992).

### Example 1: Establishment of Methods and Systems for Ex Vivo Brain Slice Culture Model of Chronic Neuroinflammation

Described herein is a novel ex vivo assay system and method, using organotypic cortical slices (OGCS), that models chronic neuroinflammation found in humans and is useful for drug screening. The systems and methods herein allow the OGCS system to survive and partially recover through the initial injury phase (recovery phase, e.g., DIV1-4), followed by a change in conditions that allow the establishment of a stable neuroinflammatory state that bares the hallmarks of neuroinflammation found in humans diseases including AD, HD, MS, ALS, FTD, PSP, etc. (stress phase, e.g., DIV 5+). Hallmarks of inflammation include the expression of key genes and secretion of key proteins in the early (acute injury, DIV1-4) phase and the late (chronic inflammation, DIV>5) phase.

Materials and Methods

C57BL/6 mice (Taconic) at postnatal day 21 were euthanized and their brains removed. The hindbrain was resected and discarded. Transverse sections at 300 μm thickness were obtained, then cultured in a “recovery” cell culture medium comprising Neurobasal A (ThermoFisher Scientific), 20% heat-inactivated horse serum, 10 mM glucose, and 0.8% antibiotics/antimycotics cocktail (ThermoFisher Scientific). After a complete media change on DIV1, the cultured slices were subjected to 50% culture media exchange every other day during this period.

On DIV4, cultured slices were switched to a “stress” culture media comprising Neurobasal A, 1% B27, 10 mM glucose, and 0.8% antibiotics/antimycotics cocktail. The cultured slices were subjected to 50% culture media exchange every other day, with the “stress” culture media.

### Example 2: Brain Slice Culture Model Exhibits Maintained Viability

Materials and Methods

The viability of the brain slice culture model is assessed using a LIVE/DEAD cell viability assay (ThermoFisher Scientific) where the brain slice is dissociated and cells are exposed to dyes that are either membrane permeable or impermeable, permitting interrogation of membrane integrity and cell viability. Transcriptomic data are also used to identify the change or loss of expression of cell type specific transcripts as a representation of the cell populations present in the brain slice.

### Results

A substantial loss of all cell types occurs between day 0 and day 3 in culture, with the largest drop occurring during the first 24 hours in culture. The total cell numbers then remain relatively stable from day 4 up to 14 in culture. Transcriptomic assessment of cell type specific gene expression indicates that all cell types are lost early in the culture process, but remain stable after DIV4 and in the absence of serum.

### Example 3: Brain Slice Culture Model Maintains Adult Microglia and Other Mature CNS Cell Populations in Culture

Materials and Methods

The presence of microglia in the cultured brain slices was assessed using two methods. Microglia were purified from dissociated brain slices using CD11b microbeads (Miltenyi Biotec). RNA or protein was purified from the isolated cells from day 0 up to day 14 in culture. The presence of microglia in the slice was also evaluated using immunohistochemical staining for CD68 (AbD Serotec), a lysosomal marker known to increase expression under inflammatory conditions.

Results

Gene expression specific to various CNS cell types including, neurons, microglia, astrocytes, and oligodendrocytes, can be detected and analyzed from the brain slices grown in stress culture media. Microglia-specific transcriptomic data was obtained from the purified microglia from cultures brain slices. CD68-positive microglia were detected in slices (immunohistochemistry data not shown), thereby indicating a microglial inflammatory state and proliferation. An increasing number of astrocytes is corroborated by the increased Gfap gene expression, as well as increased GFAP protein expression that has been observed over time in the platform (data not shown). NeuN and/or MAP2 immunostaining was used to visualize the presence of surviving neurons in the cultured brain slice (immunohistochemistry not shown), but the number of healthy neurons declined from DIV0 to DIV14.

### Example 4: Brain Slice Culture Model Exhibits Hallmarks of Acute and Chronic Neuroinflammation, as Determined by Cytokine Secretion

The pro-inflammatory cytokines IL-6 and TNF-α have been shown to be elevated in postmortem brains from Huntington's Disease and Alzheimer's Disease patients. See: Chen et al., Cerebrospinal Fluid Inflammatory Cytokine Aberrations in Alzheimer's Disease, Parkinson's Disease and Amyotrophic Lateral Sclerosis: A Systematic Review and Meta-Analysis, Frontiers in Immunology, 9 (2018), Yang et al., Microglial Activation in the Pathogenesis of Huntington's Disease, Frontiers in Aging Neuroscience, 9 (2017), Taipa et al., Proinflammatory and anti-inflammatory cytokines in the CSF of patients with Alzheimer's disease and their correlation with cognitive decline, Neurobiology of Aging, 76 (2019), Bjorkqvist et al., A novel pathogenic pathway of immune activation detectable before clinical onset in Huntington's disease, Journal of Experimental Medicine, 205(8) (2008). IL-6 and TNF-α secretion was assessed in the cultured brain slices to determine whether the brain slice culture model recapitulates the increased cytokine secretion phenotype found in Huntington's Disease and Alzheimer's Disease.

Materials and Methods

Conditioned media was collected between days 1-9 in cultures and stored frozen (−80° C.) until analyzed. CSF from 7-10-week-old mice was obtained from equal numbers of male and female mice. CSF was then pooled and aliquoted and stored frozen. Conditioned media was used to measure levels of a panel of secreted proteins. Media and CSF were analyzed using the FirePlex® Mouse Discovery Cytokines-Immunoassay Panel to quantify 70 individual cytokines.

Results

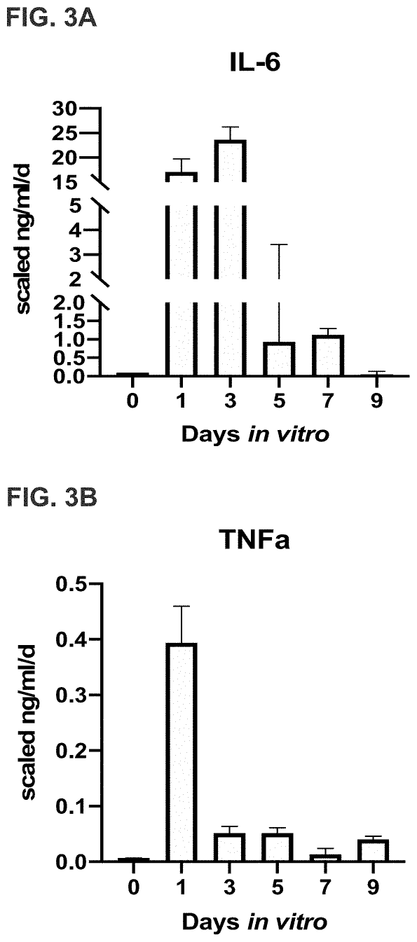

Cytokine secretion from the cultured slices were compared to naïve murine cerebrospinal fluid at baseline (Day 0). Results for IL-6 secretion are depicted in FIG. 3A (mean+SEM). Naïve murine CSF exhibited [0.093±0 ng/ml/d] IL-6. The cultured brain slices demonstrated a spike in IL-6 secretion on DIV 1 [17.13±2.62] and DIV 3 [23.65±2.62]. The cultured brain slices also demonstrated increased IL-6 secretion on DIV 5 [0.94±2.47] and DIV 7 [1.13±0.17], as compared to baseline CSF.

Results for TNF-α secretion are depicted in FIG. 3B (mean+SEM). Naïve murine CSF exhibited [0.007±0.001 ng/ml/d] TNF-α. The cultured brain slices demonstrated a spike in TNF-α secretion on DIV 1 [0.39±0.07] and increased TNF-α secretion as compared to naïve CSF on DIV 3 [0.05±0.01]. The cultured brain slices also demonstrated increased TNF-α secretion on DIV 5 [0.05±0.01], DIV 7 [0.01±0.01], and DIV 9 [0.04±0.01] compared to baseline CSF.

Assessment of IL-6 and TNF-α secretion in the cultured brain slices demonstrated that the brain slice culture model recapitulates the increased cytokine secretion phenotype found in Huntington's Disease and Alzheimer's Disease.

### Example 5: Brain Slice Culture Model Exhibits a Reproducible Transcriptomics Signature Over Time

Materials and Methods

Slice Culture Harvest and Methods

Brain slices were obtained from C57BL/6 mice and cultured as described in Example 1.

RNA Isolation and Analysis

RNA was isolated from cultured slices on DIV0, DIV1, DIV3, DIV4, DIV6, DIV8, DIV11, and DIV14. Isolated RNA was first examined for quality using the Agilent RNA 6000 Nano kit according to manufacturer's instructions. Any sample with an RNA integrity number (RIN) less than 7 was not used for downstream analysis. RNA was quantified using a Nanodrop ONE spectrophotometer. RNA was then analyzed using the nCounter Nanostring neuropathology panel and mouse immunology panel (plus 30 custom spike in genes) according to manufacturer's instructions.

100 ng of RNA used to hybridize to Nanostring probe sets; mouse Neuropathology or mouse Immunology plus 30 custom genes.

Transcriptomics Data Analysis

All transcriptomic data were processed and analyzed using the nSolver software (NanoString). Every panel contained 8-10 negative controls that were used to estimate noise levels. Counts determined to be below the noise threshold were assigned the value designated to the noise cutoff. Genes whose expression did not exceed noise levels in any treatment group were not included in any statistical tests. T-tests, false discovery rate statistics, and volcano plots were computed using Prism (Graphpad). Pathway analysis of gene lists was conducted using GProfiler (https://biit.cs.ut.ee/gprofiler/gost). Differential gene expression was correlated between the human neuroinflammatory subpopulation and the brain slice culture using parametric statistics (Pearson). Statistics reported include rho, p-value of correlation, r₂, and when relationship of the correlation is informative, slope of the best-fit line.

Results

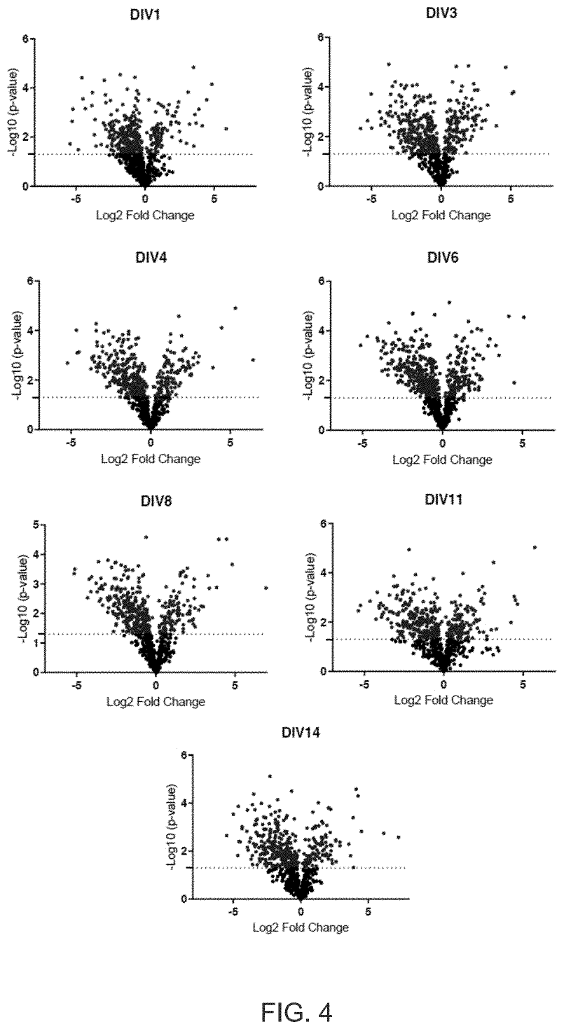

Transcriptomic changes in the cultured slices were evaluated longitudinally at DIV0, 1, 3, 4, 6, 8, 11, and 14. See Tables 5-11 (listing differentially expressed genes at DIV 1, 3, 4, 6, 8, 11, or 14 v. DIV 0). A large number of the total 770 genes were significantly altered at each time point: 358 genes at DIV1, 437 genes at DIV3, 389 genes at DIV4, 459 genes at DIV6, 400 genes at DIV8, 372 genes at DIV11, and 366 genes at DIV14. As seen in FIG. 4, a large number of genes were differentially expressed over time. Interestingly, some of the most highly upregulated genes over time include genes involved in matrix remodeling (Mmp12), oxidative stress (Hmox1), and cell surface interactions (Cd44). The most downregulated genes are involved in microglial homeostasis (P2ry12, Tmem119), as well as, molecules involved in healthy neuron-microglia communication (Cx3cl1). As the slices remained in culture for longer periods of time, increased numbers of neuronal signaling genes were downregulated (for example, GABA receptors Gabra1, Gabrb2) while matrix remodeling (Mmp12) and complement related genes (C3) were upregulated.

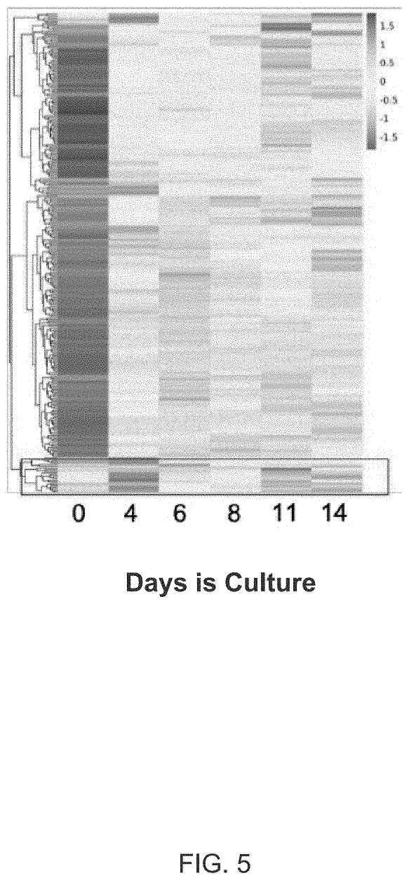

Hierarchical clustering indicated a dramatic change between DIV0 and DIV4, which change was maintained through DIV14 (FIG. 5). A small cluster of genes continued to change between DIV4 to DIV14 (red box, FIG. 5). KEGG pathway analysis on these 23 genes in the small cluster indicated pathways such as PI3K-AKT signaling, MAPK signaling, and cell adhesion molecules continue to decline from DIV4-DIV14 (Tables 12-18). Overall, the brain slice culture model exhibited a reproducible gene expression pattern over time.

### Example 6: Brain Slice Culture Model Transcriptomics Correlates with Human Alzheimer's Disease Transcriptome

To assess whether the gene expression changes observed in the cultured brain slices correlated with gene expression changes attributed to Alzheimer's disease (AD) in humans, the alterations in gene expression in the culture model were compared to gene expression patterns observed in AD brains.

Materials and Methods

Slices were cultured and harvested as described in Example 1. RNA was isolated and transcriptomics data analyzed as described in Example 5.

The slice culture transcriptomics data was analyzed and compared to a subpopulation of patients in the Religious Orders Study and the Memory and Aging Project (ROSMAP) RNAseq data characterized by a having a neuroinflammatory signature (De Jager et al. Scientific Data 2018), which is publicly available through AMPAD via SAGE bionetworks. The genes that overlapped between the NanoString platform and the RNA-seq data from ROSMAP were compared in terms of their differential expression. The strength and significance of association was measured via Spearman rank correlation.

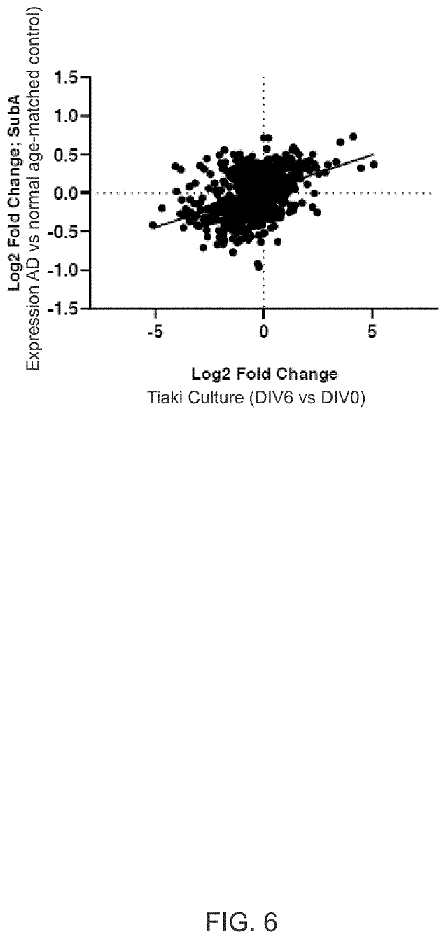

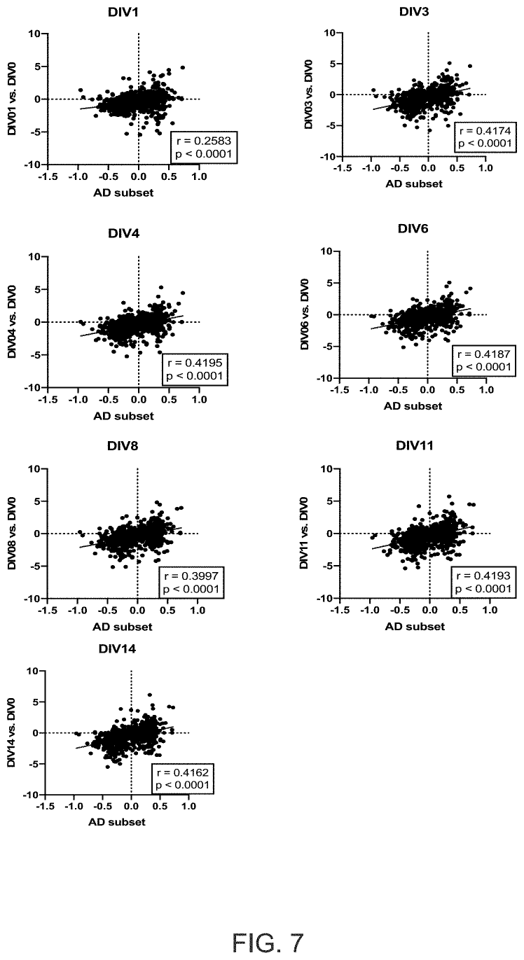

Results are shown in FIG. 6. Differential gene expression data for the cultured brain slice model is shown as Log 2 fold change between DIV0 and DIV6 (Y-axis). Results for each of the DIV time points sampled are shown in FIG. 7 The SubA data represents differential expression between age-matched control and AD brain (X-axis). Each data point represents differential expression of an individual gene. There was statistically significant positive correlation between the two datasets (Pearson correlation coefficient rho=0.4201, p<0.0001). The results suggested a significant association between the cultured brain slice ex vivo AD signature and the neuroinflammatory subpopulation of human AD signature.

Stabilization of Neuropathology Signature Over Time

Expression levels at each time point were compared to the expression profile at DIV6, as DIV6 was determined to align with human AD. This data should allow us to understand at what time point the disease profile has stabilized, as well as inform the end user as to what time point to initiate compound treatment for either preventative intervention or therapeutic intervention. As seen in FIG. 7, correlation coefficients did not stabilize at 0.95 until DIV4. This suggests that the chronic, disease associated inflammatory profile did not stabilize until DIV4. Therefore, any treatments initiated before DIV4 would be considered preventative. Likewise, treatments initiated at DIV4 or later would be considered ameliorative.

### Discussion

The brain slice culture model demonstrated dramatic changes in the transcriptome. When compared to DIV0 profiles, 350-500 genes significantly changed depending on the timepoint. Many of the genes changing reflect the biology of the slice culture platform process. Genes involved in stress and cytokine release were increased, due to the stress and inflammation that occurs during the process of preparing and plating the brain slices. Conversely, genes involved in neuronal signaling and tissue integrity were downregulated, as the cultured brain slices underwent neurodegenerative processes.

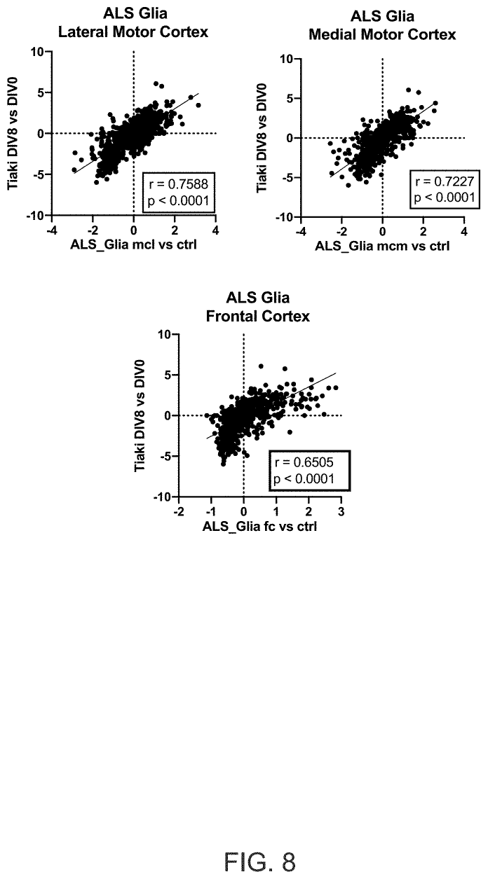

Independent bioinformatic analysis indicates that the brain slice culture model disclosed herein correlates with a subpopulation of the human Alzheimer's disease ROSMAP data set, with a spearman's correlation coefficient (rho) of 0.35 and a p-value of <0.01. This alignment with human AD begins at DIV4, as DIV4, DIV6, DIV8, DIV11, and DIV14 all highly correlate (rho>0.94) (FIG. 8). Therefore, in the brain slice culture model, it is possible to modulate targets of interest in both preventative (prophylactic) and therapeutic (e.g., ameliorative) treatment paradigms.

Overall, the transcriptome from the brain slice culture model was dynamic, as indicated by longitudinal changes in multiple biological processes. In addition, gene changes were found to correlate with human AD patient RNAseq profiles. The time course of the cultured brain slice model transcriptomic profile not only reflects neurodegenerative processes that would be expected to occur in the brain, but correlates with human disease. The alignment with a subpopulation of the ROSMAP patients indicates that the brain slice culture model disclosed herein is suitable for target identification and validation campaigns to enable drug discovery activities to identify a disease-modifying therapeutic for the treatment of neuroinflammatory AD.

### Example 7: Brain Slice Culture Model Aligns with Transcriptome of Human ALS Patient Subgroups with a Glial Activation Signature

A recent transcriptomics analysis of ALS postmortem cortex samples identified three subpopulations with distinct molecular subtypes of ALS: a subtype associated with glial activation signature, a subtype associated with oxidative and proteotoxic stress, and a subset associated with TARDBP/TDP-43 dysfunction. Tam et al., Cell Reports 29, 1164-1177, Oct. 29, 2019, which is hereby incorporated by reference in its entirety. To assess whether the gene expression changes observed in the cultured brain slices correlated with gene expression changes attributed to ALS or to particular ALS subtypes in humans, the alterations in gene expression in the culture model were compared to gene expression patterns observed in the ALS subgroups described in Tam et al.

Materials and Methods

Human ALS Data Sets

Data from two publicly available datasets (GSE122649 and GSE124439) were used to identify subpopulations of ALS patients. See Tam et al., Postmortem Cortex Samples Identify Distinct Molecular Subtypes of ALS: Retrotransposon Activation, Oxidative Stress, and Activated Glia, Cell Reports 29 (2019). Data files and required meta data was acquired and transformed from GEO. The data were processed and quality controls were assessed. The data were filtered and normalized using limma voom methodology. Differential expression was determined between healthy controls and ALS patients. We utilized the subpopulations as defined by Tam et al. to determine alignment with the Tiaki neuroinflammatory signature.

Generation of Slice Culture Whole Tissue RNA

Slices were cultured and harvested as described in Example 1. RNA was isolated and transcriptomics data analyzed as described in Example 6.

RNA Sequencing and Differential Expression Analysis of Slice Culture Whole Tissue RNA

RNA sequencing and initial QC was performed at Insight Genetics (Nashville, Tenn.). Resulting Fastq files were transferred to Immuneering for full QC, count transformation and normalization, and quantification of differential gene expression (neuroinflamed vs. reference). The limma voom methodology was used to transform data to logged counts per million and Trimmed Mean of M values (TMM) was used for inter-sample normalization. Limma voom was used to perform differential expression analysis [see Ritchie et al., limma powers differential expression analyses for RNA-sequencing and microarray studies, Nucleic Acids Research 43(7) (2015)].

Results

Results for the ALS Glial Activation subgroup are shown in FIG. 8. Differential gene expression data for the cultured brain slice model is shown as Log 2 fold change between DIV0 and DIV8 (Y-axis). The ALS Glial Activation subpopulation data from lateral motor cortex, medial motor cortex, and frontal cortex represents differential expression between age-matched control and the applicable ALS subpopulation. Each data point represents differential expression of an individual gene.

As shown in FIG. 8, there was significant correlation between the cultured brain slice model dataset and the ALS Glial Activation lateral motor cortex dataset (r=0.7588, p=<0.0001). There was also significant correlation between the cultured brain slice model dataset and the ALS Glial Activation medial motor cortex dataset (r=0.7227, p=<0.0001). There was also significant correlation between the cultured brain slice model dataset and the ALS Glial Activation frontal cortex dataset (r=0.6505, p=<0.0001).

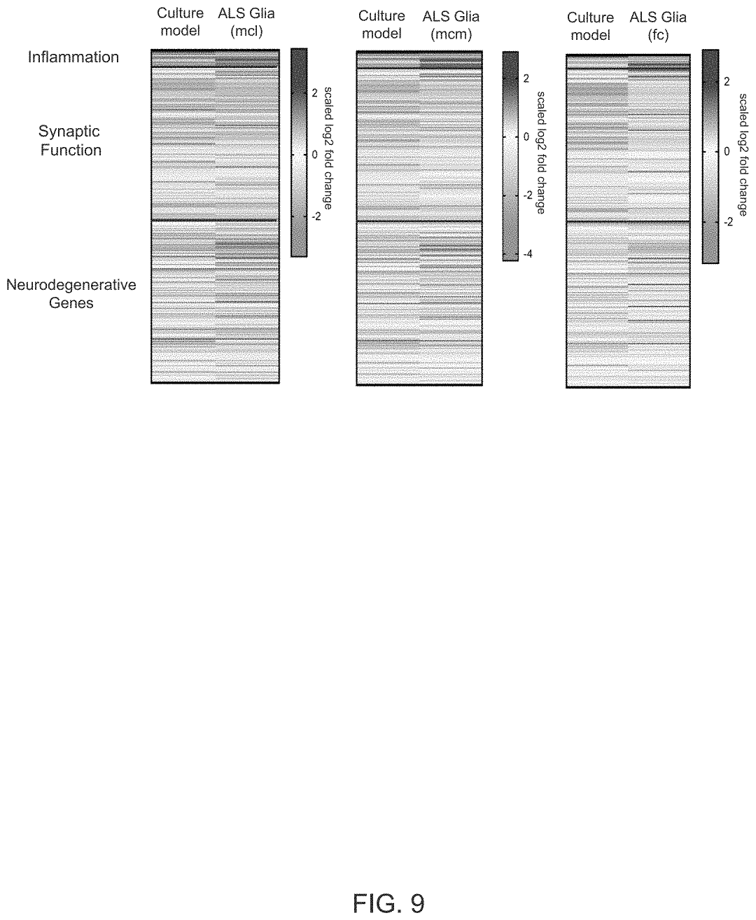

FIG. 9 depicts the alignment of the brain slice culture model signature with the transcriptomics signature driven by inflammatory, synaptic function, and neurodegeneration-related genes in the ALS Glial Activation subgroup. As shown in FIG. 9, the brain slice culture model aligned with the ALS Glial Activation subgroup, as shown by increased gene expression of inflammatory genes (including C3, Hmox1, C1qc which have a ˜2-fold change in ALS and Tiaki datasets), decreased gene expression of genes involved in synaptic function (including Calb1, Gabrb2, Thy1 which have a ˜2-fold change in ALS and Tiaki datasets), and increased expression of genes linked to neurodegenerative disease (including Cd44, Gfap, Spi1 which have a ˜2-fold change in ALS and Tiaki datasets). These functional subclusters were defined by gene function using gProfiler analysis.

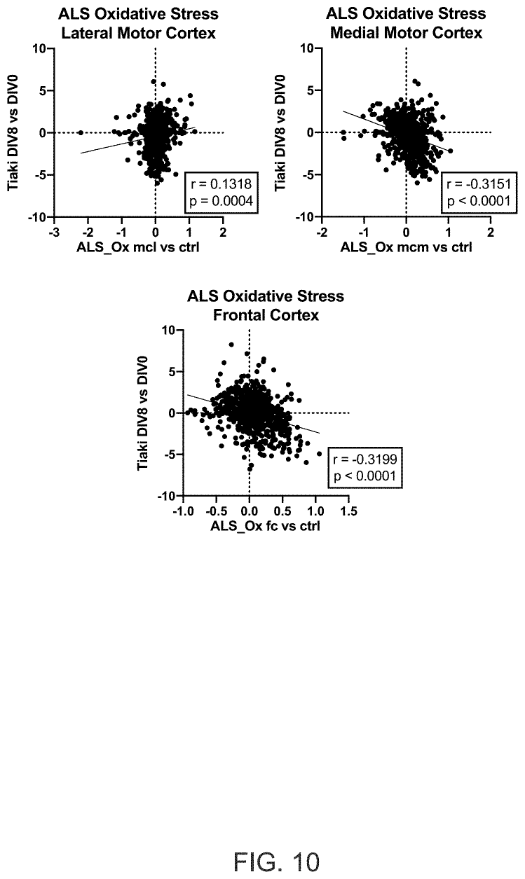

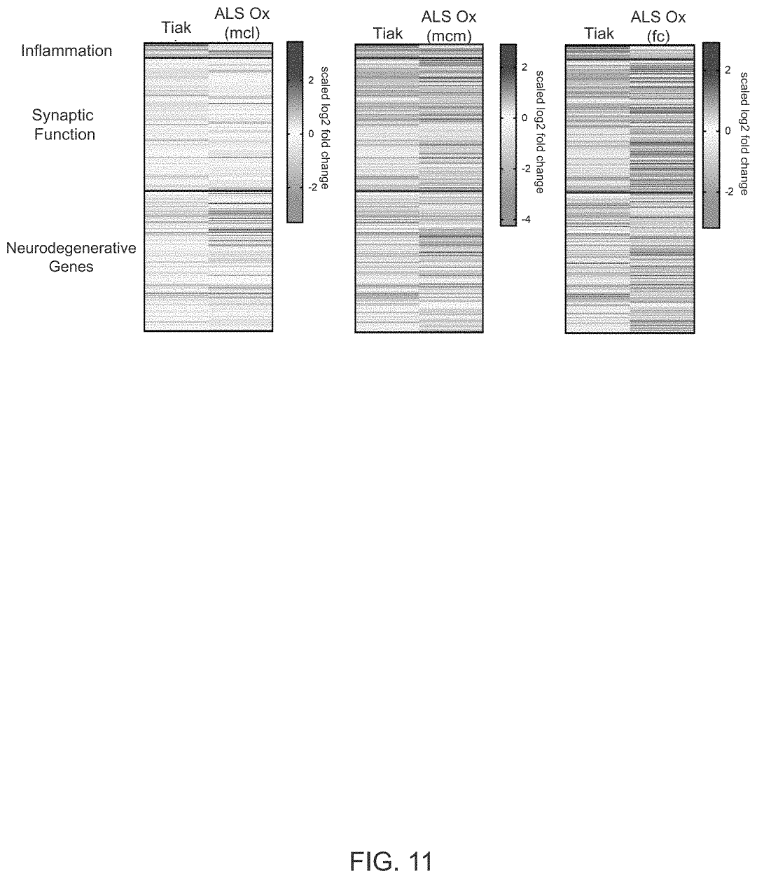

Results for the ALS Oxidative Stress patient subgroup are depicted in FIGS. 10 and 11. The transcriptomics signature for the brain slice culture model did not align with the ALS Oxidative Stress subgroup, which was enriched for genes associated with cellular stress pathways. These results indicate that cellular stress responses seen in the ALS Oxidative Stress patient subgroup did not drive the biology that was characteristic of the brain slice culture model.

While the invention has been particularly shown and described with reference to a preferred embodiment and various alternate embodiments, it will be understood by persons skilled in the relevant art that various changes in form and details can be made therein without departing from the spirit and scope of the invention.

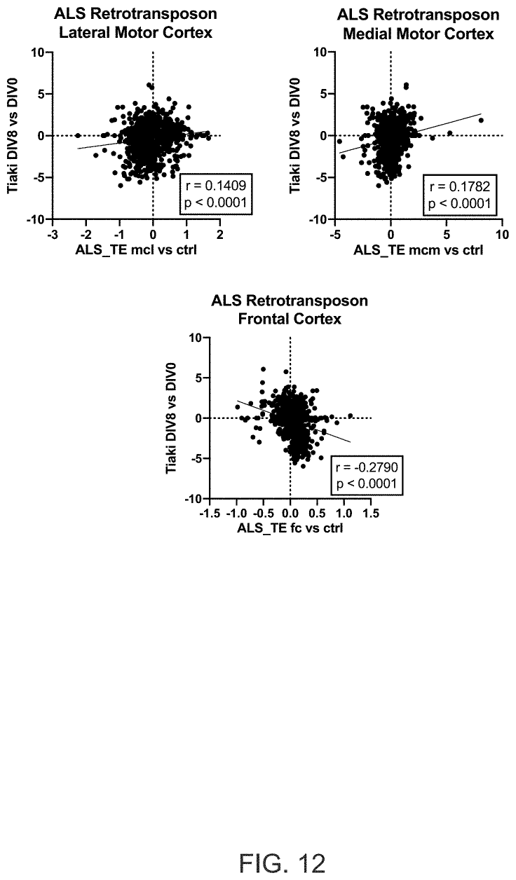

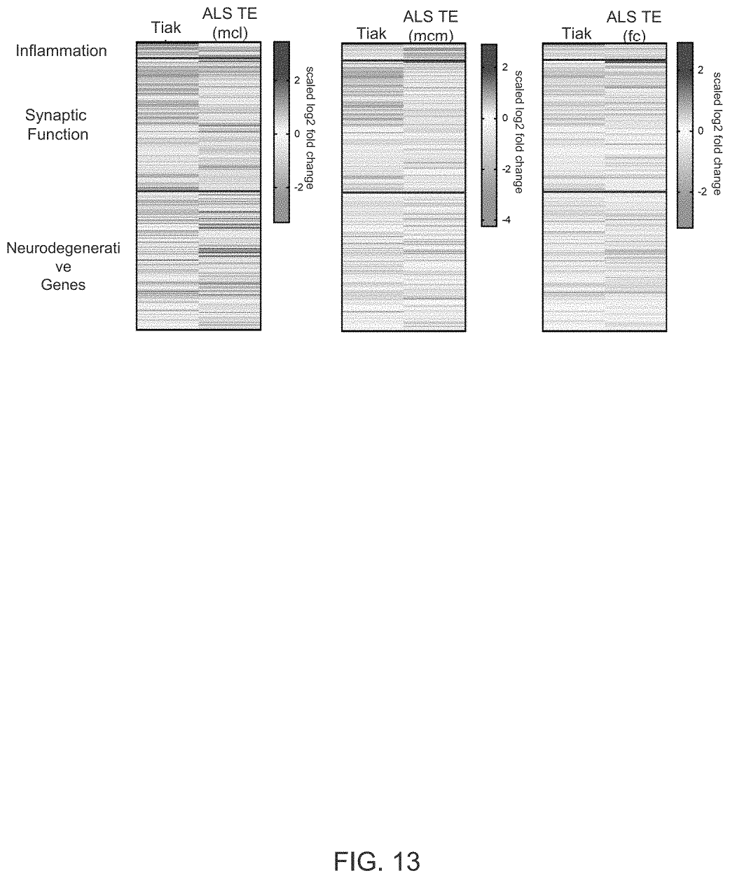

Results for the ALS TARDBP/TDP-43 (retrotransposon-related) patient subgroup are depicted in FIGS. 12 and 13. The transcriptomics signature for the brain slice culture model did not align with the TARDBP/TDP-43 subgroup.

### Example 8: Brain Slice Culture Model Aligns with Transcriptome of Brain Regions Exhibiting Neurodegeneration in Human Huntington's Disease Patients

To assess whether the brain slice culture model described herein is a suitable model for Huntington's disease, the whole tissue transcriptome from the slice cultures was compared to the caudate nucleus of Huntington's disease patients.

Materials and Methods

Human Huntington's Disease Patient Datasets

Publicly available datasets were evaluated for data quality, patient number, and available metadata for patient samples. Selected datasets included: GSE3790 (described in Hodges A et al., 2006, which is hereby incorporated by reference in its entirety). GSE3790 comprises an expression profiling by array of 4 brain regions, cerebellum, frontal cortex (BA9 and BA4), and caudate nucleus, for 36 control and 44 HD patient brain samples. Selected datasets also include GSE26927 (Durrenberger et al., 2015, which is hereby incorporated by reference in its entirety). GSE26927 comprises an expression profiling by array of 10 control and 10 HD patient brain samples from the ventral caudate. Data analysis was performed by BISC Global—raw data files were acquired from GEO, as well as any meta data. The data was processed to assess quality control, background correction, count normalization (rma) and transformation, followed by quantification of differential gene expression (HD vs Control). For GSE3790, analysis for all 4 brain regions was performed separately.

Generation of Tiaki Slice Culture Whole Tissue RNA

Tiaki slice cultures were created and maintained as described herein. After establishment of the chronic neuroinflammatory state, slices were collected and processed to RNA. Each RNA preparation was derived from a mixture of cultures that were balanced for gender. A total of 5 preparations were submitted for RNA sequencing. Brain tissue that was chopped and immediately processed to RNA served as the reference sample (DIV 0).

RNA Sequencing and Differential Expression Analysis

RNA sequencing and initial QC was performed at Insight Genetics (Nashville, Tenn.). Resulting Fastq files were transferred to Immuneering for full QC, count transformation and normalization, and quantification of differential gene expression (neuroinflamed vs. reference). The limma voom methodology was used to transform data to logged counts per million and Trimmed Mean of M values (TMM) was used for inter-sample normalization. Limma voom was used to perform differential expression analysis [see Ritchie et al., limma powers differential expression analyses for RNA-sequencing and microarray studies, Nucleic Acids Research 43(7) (2015)].

Alignment of Tiaki Transcriptome to HD Patient Transcriptome

The comparison was carried out based on the overlapping gene ID's between the brain slice culture RNAseq and HD data sets. Gene sets were compared based on significance; all genes, genes with a significant p-value (cut-off 0.05), or genes with a significant adjusted p-value (cut-off 0.05). A non-parametric Kendall correlation was used to compare the brain slice culture and HD data sets. Heat map visualizations of the dysregulation observed in the gene sets associated with HD were created by assigning a score of 1 or −1 to genes that exhibited significant (adj p<=0.05) up or downregulation, respectively. Genes that exhibited trends (adj p<=0.15) for up or downregulation were assigned a score of 0.5 or −0.5, respectively. No statistical analyses were performed using these scores.

Results

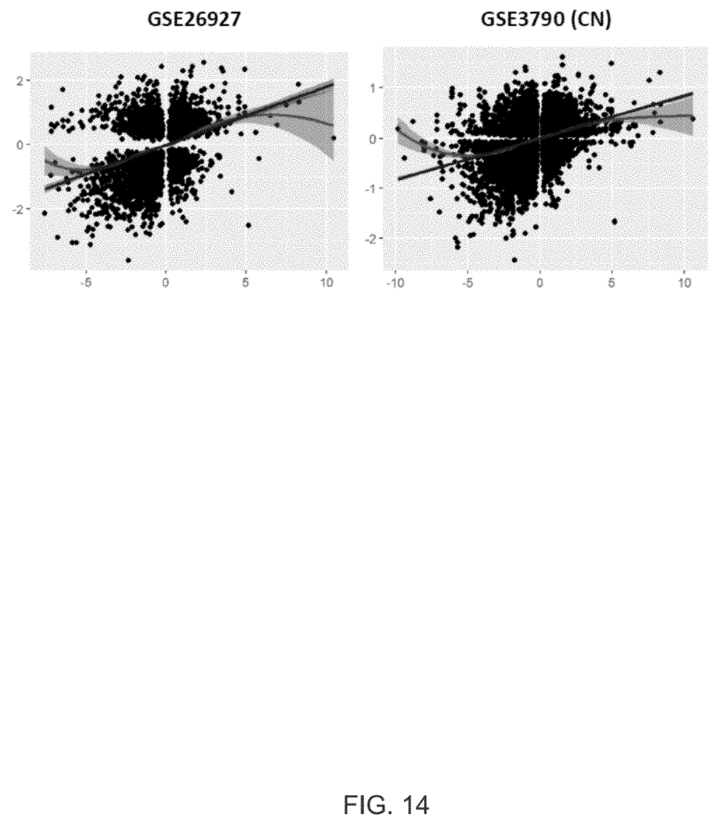

Comparison of gene signatures using the false discovery rate (FDR) adjusted p-value cut off for differential gene expression (log 2), the brain slice culture transcriptome exhibited a modest, but significant alignment with the transcriptome from the Huntington's Disease patients (FIG. 14). The overlap of the brain slice culture transcriptome was similar for the two datasets; GSE26927 (Kendall τ coefficient=0.208, p<0.01) compared with the caudate nucleus dataset from GSE3790 (Kendall τ coefficient=0.216, p<0.01).

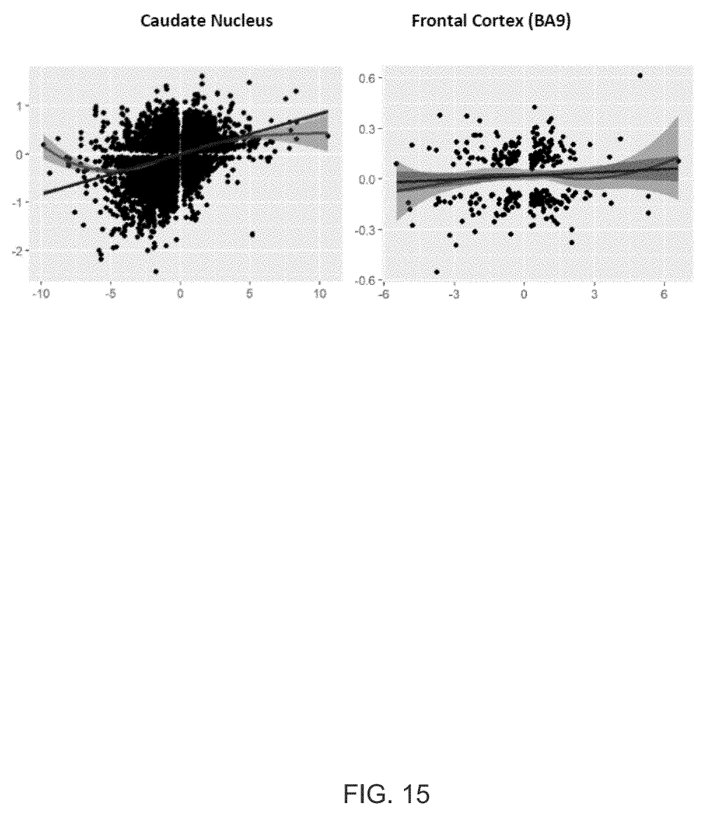

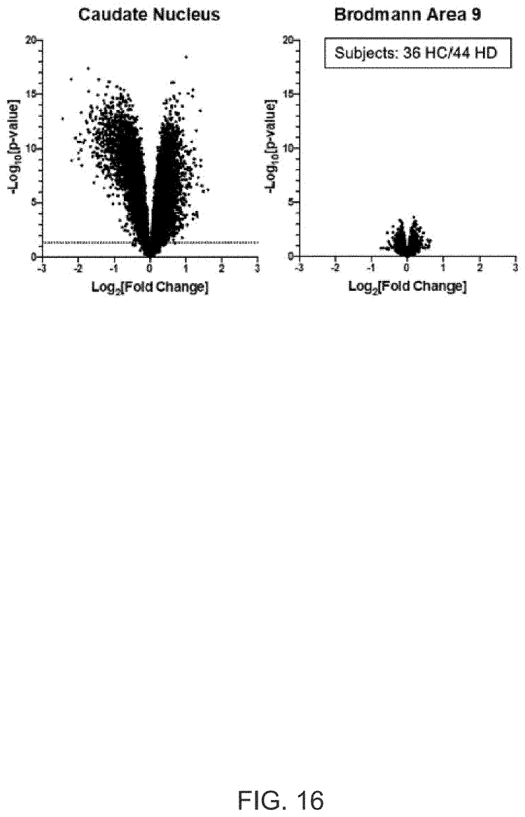

The brain region exhibiting neuronal death, network dysfunction, and the greatest pathology load for HD is the caudate nucleus. Interestingly, this is the brain region with the greatest alignment of gene dysregulation between the brain slice culture transcriptome and HD patients (FIG. 15). The correlation between the Tiaki whole tissue transcriptome is greater with the caudate nucleus (Kendall τ coefficient=0.216, p<0.01) compared with the frontal cortex (BA9; Kendall τ coefficient=0.0324, p>0.05). Interestingly, none of the log 2 fold changes for BA9 achieved the FDR statistical cutoff. Therefore, a p-value cutoff of 0.05 was used in order to compare the brain slice culture and BAD HD dataset. This lack of significant changes is likely due to the lack of pathology in the BA9 brain region in HD, thereby resulting in less deviation from control subjects (FIG. 16).

### Example 9: Brain Slice Culture Model Aligns with Shared Transcriptomic Neuroinflammatory Disease Signature Across Various Neurodegenerative Diseases

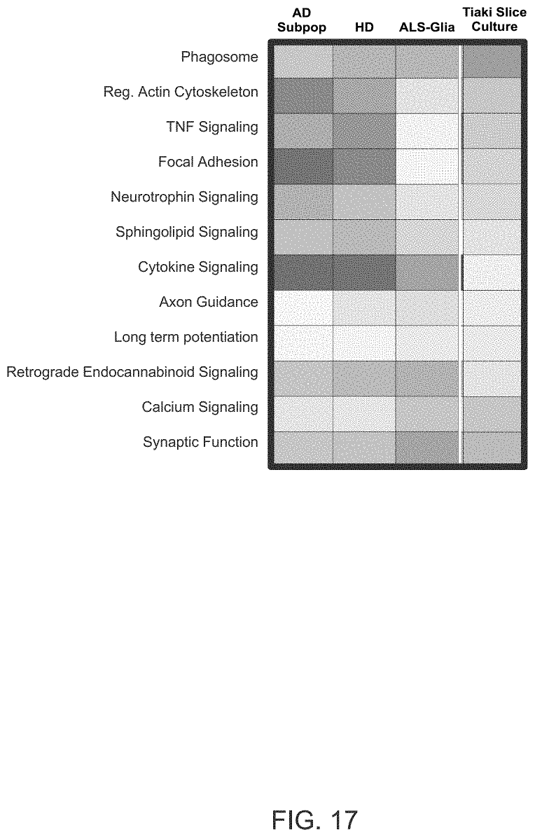

A gene set enrichment analysis (GSEA) was performed using the Kegg pathways in order to determine overlap of dysfunctional biological pathways between the cultured brain slices, HD patients, and the AD and ALS patient subpopulations. Interestingly, multiple pathways demonstrated similar dysregulation (FIG. 17). Overall, there is significant overlap between the dysfunctional biology associated with CNS neuroinflammation associated with various neurodegenerative diseases. This dysfunctional biology is modeled by the Tiaki brain slice culture system.

### Example 10: Brain Slice Culture Alignment with Human Neuroinflammatory Signature can be Reversed Through Modulation of Molecular Targets

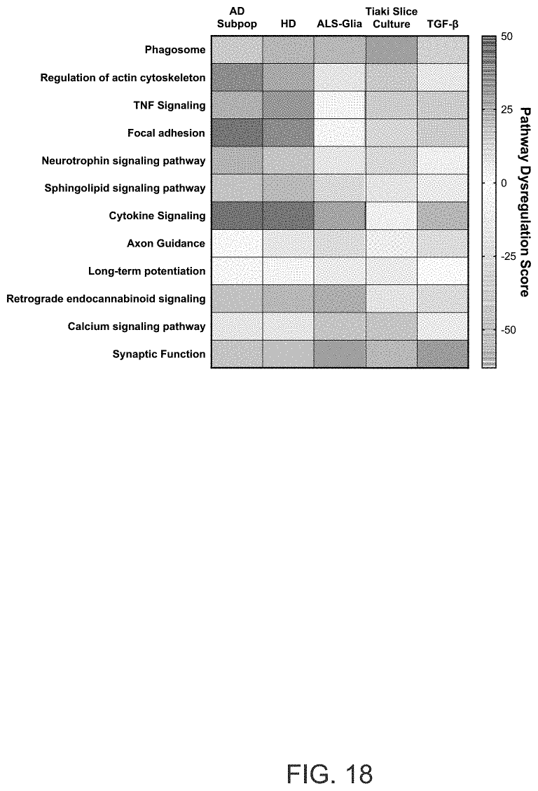

The human neuroinflammatory state modeled by the Tiaki brain slice cultures can be reversed through the modulation of molecular targets of interest that result in a decrease in inflammatory gene expression and increases in synaptic function gene expression. For example, treatment with recombinant msTGFβ in a therapeutic paradigm (DIV5-8) reverses the dysfunctional biological pathways associated with the shared human CNS disease neuroinflammatory signature (FIG. 18). This indicates the utility of the Tiaki slice culture system in validating molecular targets of interest for the development of therapeutics that ameliorate neuroinflammation.

All references, issued patents and patent applications cited within the body of the instant specification are hereby incorporated by reference in their entirety, for all purposes.

### Supplemental Tables

TABLE 5
From TTPB_0400_final document,
Differentially Expressed Genes-DIV01

| Gene Name | Accession # | Pval |
|---|---|---|
| Hmoxl | NM_010442.2 | 0.0000 |
| Cntnl | NM_001159647.1 | 0.0000 |
| Src | NM_001025395.2 | 0.0000 |
| AI464131 | NM_001085515.2 | 0.0000 |
| Cxcl12 | NM_021704.3 | 0.0000 |
| Cd44 | NM_009851.2 | 0.0001 |
| Lrp1 | NM_008512.2 | 0.0001 |
| Ppp2r5e | NM_012024.2 | 0.0001 |
| Ngf | NM_001112698.1 | 0.0001 |
| Clu | NM_013492.2 | 0.0002 |
| Crebbp | NM_001025432.1 | 0.0002 |
| Hgf | NM_010427.5 | 0.0002 |
| Arsa | NM_009713.4 | 0.0003 |
| Fcrls | NM_030707.3 | 0.0003 |
| Gss | NM_008180.1 | 0.0003 |
| Angpt2 | NM_007426.3 | 0.0003 |
| Abat | NM_001170978.1 | 0.0003 |
| Galc | NM_008079.3 | 0.0003 |
| Cxcr4 | NM_009911.3 | 0.0004 |
| Entpd2 | NM_009849.2 | 0.0004 |
| Aars | NM_146217.4 | 0.0004 |
| Dgkb | NM_178681.4 | 0.0005 |
| Lmna | NM_001002011.2 | 0.0005 |
| Ddit3 | NM_007837.3 | 0.0005 |
| Apoe | NM_001305844.1 | 0.0005 |
| Nf1 | NM_010897.2 | 0.0005 |
| Csf1 | NM_001113530.1 | 0.0006 |
| Itga5 | NM_001314041.1 | 0.0006 |
| Slc1a2 | NM_001077514.3 | 0.0007 |
| Tmem119 | NM_146162.2 | 0.0007 |
| Tnc | NM_011607.1 | 0.0007 |
| Fmr1 | NM_008031.2 | 0.0008 |
| Man2b1 | NM_010764.2 | 0.0008 |
| Pecam1 | NM_008816.2 | 0.0008 |
| Cdkn1a | NM_007669.4 | 0.0009 |
| Lars | NM_134137.2 | 0.0009 |
| Ap4s1 | NM_021710.3 | 0.0010 |
| Stx2 | NM_007941.2 | 0.0011 |
| Bax | NM_007527.3 | 0.0011 |
| I16 | NM_031168.1 | 0.0012 |
| Nrxn1 | NM_001346960.1 | 0.0012 |
| Negr1 | NM_001039094.2 | 0.0013 |
| Nes | NM_016701.3 | 0.0014 |
| Itpr2 | NM_010586.1 | 0.0014 |
| Erlec1 | NM_025745.3 | 0.0014 |
| Rapgef2 | NM_001099624.2 | 0.0014 |
| Tnfrsf10b | NM_020275.3 | 0.0014 |
| Igf1r | NM_010513.2 | 0.0016 |
| Nova1 | NM_021361.1 | 0.0017 |
| Flt1 | NM_010228.3 | 0.0018 |
| Gnptg | NM_172529.3 | 0.0019 |
| Lama2 | NM_008481.2 | 0.0019 |
| Sox9 | NM_011448.4 | 0.0020 |
| Insr | NM_010568.2 | 0.0021 |
| Ccdc127 | NM_024201.3 | 0.0021 |
| Ncf1 | NM_001286037.1 | 0.0021 |
| P2ry12 | NM_027571.3 | 0.0023 |
| Sirt7 | NM_153056.2 | 0.0023 |
| Spast | NM_016962.2 | 0.0023 |
| Syt1 | NM_009306.2 | 0.0023 |
| Cadm3 | NM_053199.3 | 0.0024 |
| Msn | NM_010833.2 | 0.0024 |
| Ppt1 | NM_008917.3 | 0.0024 |
| Ccnd1 | NM_007631.1 | 0.0025 |
| Slc9a6 | NM_172780.3 | 0.0025 |
| Hexb | NM_010422.2 | 0.0027 |
| Grin2c | NM_010350.2 | 0.0027 |
| Gnb5 | NM_138719.5 | 0.0027 |
| Tnfrsf12a | NM_001161746.1 | 0.0027 |
| Tnf | NM_013693.2 | 0.0027 |
| Ggt1 | NM_008116.2 | 0.0028 |
| Icam2 | NM_010494.1 | 0.0028 |
| Gsr | NM_010344.4 | 0.0029 |
| Ptgs2 | NM_011198.3 | 0.0030 |
| Ube2n | NM_080560.3 | 0.0030 |
| Clqc | NM_007574.2 | 0.0030 |
| B4galt6 | NM_019737.2 | 0.0031 |
| Xiap | NM_009688.2 | 0.0031 |
| Myc | NM_010849.4 | 0.0031 |
| Kcnb1 | NM_008420.3 | 0.0032 |
| Cers1 | NM_138647.3 | 0.0033 |
| Gabrb2 | NM_008070.3 | 0.0033 |
| Map2k1 | NM_008927.3 | 0.0033 |
| Adrb2 | NM_007420.2 | 0.0034 |
| Lif | NM_008501.2 | 0.0035 |
| Cdk5r1 | NM_009871.2 | 0.0035 |
| Stat3 | NM_213659.2 | 0.0036 |
| Map2 | NM_008632.2 | 0.0037 |
| Pla2g4b | XM_925095.2 | 0.0037 |
| Gpdl1 | NM_175380.5 | 0.0038 |
| Slc12a5 | NM_020333.2 | 0.0039 |
| Prnp | NM_011170.2 | 0.0040 |
| Vegfa | NM_001025250.3 | 0.0043 |
| C1qb | NM_009777.2 | 0.0043 |
| Apaf1 | NM_009684.2 | 0.0043 |
| Lsm7 | NM_025349.2 | 0.0043 |
| Serpinb6a | NM_001164117.1 | 0.0044 |
| Trp53 | NM_011640.1 | 0.0044 |
| Sqstm1 | NM_011018.2 | 0.0044 |
| Gprasp1 | NM_001004359.2 | 0.0044 |
| Akt3 | NM_011785.3 | 0.0044 |
| Htt | NM_010414.1 | 0.0045 |
| Grm5 | NM_001143834.1 | 0.0045 |
| Mmp12 | NM_008605.3 | 0.0046 |
| Mapk10 | NM_001081567.1 | 0.0046 |
| Ran | NM_009391.3 | 0.0047 |
| Grin2b | NM_008171.3 | 0.0047 |
| Il10ra | NM_008348.2 | 0.0049 |
| Rad23b | NM_009011.4 | 0.0049 |
| Bnip3 | NM_009760.4 | 0.0049 |
| Epha7 | NM_001122889.1 | 0.0049 |
| Grin2a | NM_008170.2 | 0.0050 |
| Sgpl1 | NM_009163.3 | 0.0051 |
| Eif2s1 | NM_026114.3 | 0.0052 |
| Glrb | NM_010298.5 | 0.0052 |
| Stambpl1 | NM_029682.4 | 0.0052 |
| Rab3c | NM_023852.5 | 0.0053 |
| Pfn1 | NM_011072.4 | 0.0054 |
| Ncam1 | NM_001113204.1 | 0.0054 |
| Cd33 | NM_001111058.1 | 0.0055 |
| Cx3cl1 | NM_009142.3 | 0.0056 |
| Rela | NM_009045.4 | 0.0056 |
| Lsm2 | NM_001110101.2 | 0.0057 |
| Casp7 | NM_007611.2 | 0.0059 |
| C1qa | NM_007572.2 | 0.0059 |
| Bid | NM_007544.3 | 0.0059 |
| Cxcl16 | NM_023158.6 | 0.0060 |
| Dll4 | NM_019454.2 | 0.0062 |
| Gucy1b3 | NM_017469.4 | 0.0063 |
| Unc13a | NM_001029873.2 | 0.0064 |
| Arrb2 | NM_145429.4 | 0.0065 |
| Nptn | NM_009145.2 | 0.0065 |
| Aktls1 | NM_026270.4 | 0.0066 |
| Emp2 | NM_007929.2 | 0.0066 |
| Vcp | NM_009503.3 | 0.0066 |
| Camk4 | NM_009793.3 | 0.0067 |
| Lclat1 | NM_001177967.1 | 0.0067 |
| I14ra | NM_001008700.3 | 0.0067 |
| Gabrb3 | NM_008071.3 | 0.0068 |
| Trpm2 | NM_138301.2 | 0.0068 |
| U2af2 | NM_133671.3 | 0.0069 |
| Ppp3cc | NM_008915.2 | 0.0070 |
| Itpr1 | NM_010585.2 | 0.0070 |
| Atp8a2 | NM_015803.2 | 0.0070 |
| Col4a2 | NM_009932.3 | 0.0072 |
| Shank2 | NM_001081370.2 | 0.0073 |
| Atf4 | NM_009716.2 | 0.0073 |
| Nell2 | NM_016743.2 | 0.0074 |
| Prkce | NM_011104.2 | 0.0075 |
| Efna5 | NM_207654.2 | 0.0075 |
| Prkacb | NM_011100.3 | 0.0076 |
| Grm2 | NM_001160353.1 | 0.0079 |
| Atp6v0e2 | NM_133764.3 | 0.0080 |
| Acaa1a | NM_130864.3 | 0.0080 |
| Prpf3 | NM_027541.4 | 0.0080 |
| Nwd1 | NM_176940.5 | 0.0080 |
| Ap3m2 | NM_029505.3 | 0.0083 |
| PlCl2 | NM_013880.3 | 0.0084 |
| Prkcb | NM_008855.2 | 0.0084 |
| Calb1 | NM_009788.4 | 0.0084 |
| Inpp5f | NM_178641.5 | 0.0084 |
| Fus | NM_139149.2 | 0.0084 |
| Gabra1 | NM_010250.4 | 0.0085 |
| Kif3a | NM_008443.3 | 0.0085 |
| Ppp3ca | NM_008913.4 | 0.0085 |
| Cntnap1 | NM_016782.2 | 0.0086 |
| Ppml1 | NM_178726.3 | 0.0087 |
| Gtf2a1 | NM_175335.3 | 0.0087 |
| Dlg3 | NM_001177778.1 | 0.0087 |
| Optn | NM_181848.4 | 0.0088 |
| Sod2 | NM_013671.3 | 0.0088 |
| Taz | NM_181516.5 | 0.0090 |
| Slc8a1 | NM_011406.2 | 0.0091 |
| Dlg4 | NM_001109752.1 | 0.0091 |
| Camk2g | NM_001039138.1 | 0.0094 |
| Gdpd2 | NM_023608.3 | 0.0094 |
| Gnai3 | NM_010306.2 | 0.0094 |
| Rab2a | NM_021518.3 | 0.0095 |
| Cln8 | NM_012000.3 | 0.0098 |
| Cd68 | NM_009853.1 | 0.0099 |
| Ube2k | NM_016786.3 | 0.0099 |
| Sucla2 | NM_011506.1 | 0.0100 |
| Fos | NM_010234.2 | 0.0101 |
| Gnaq | NM_008139.5 | 0.0101 |
| Idh1 | NM_010497.2 | 0.0102 |
| Gria1 | NM_001252403.1 | 0.0102 |
| Prkca | NM_011101.3 | 0.0103 |
| Ptdss1 | NM_008959.3 | 0.0103 |
| Gabrg2 | NM_177408.5 | 0.0103 |
| Thy1 | NM_009382.3 | 0.0104 |
| Cds1 | NM_173370.3 | 0.0105 |
| Nc1 | NM_010880.3 | 0.0108 |
| Acto1 | NM_134156.2 | 0.0114 |
| Dlat | NM_145614.4 | 0.0116 |
| Hdac2 | NM_008229.2 | 0.0118 |
| Plxnc1 | NM_018797.2 | 0.0120 |
| Pcsk2 | NM_008792.3 | 0.0122 |
| Scn1a | NM_018733.2 | 0.0123 |
| Trim37 | NM_197987.2 | 0.0124 |
| Cul1 | NM_012042.3 | 0.0126 |
| Ptprn2 | NM_011215.2 | 0.0126 |
| Prkcg | NM_011102.3 | 0.0127 |
| Ikbkb | NM_010546.2 | 0.0130 |
| Gba | NM_001077411.1 | 0.0132 |
| Syt4 | NM_009308.3 | 0.0133 |
| Rab3a | NM_009001.6 | 0.0134 |
| Snca | NM_009221.2 | 0.0134 |
| Mfn2 | XM_006535920.1 | 0.0143 |
| C3 | XM_011246258.1 | 0.0143 |
| Gabra4 | NM_010251.2 | 0.0144 |
| Cd9 | NM_007657.3 | 0.0144 |
| Slc4a10 | NM_001242380.1 | 0.0146 |
| Col4a1 | NM_009931.2 | 0.0146 |
| Tada2b | NM_001170454.1 | 0.0147 |
| Ddc | NM_016672.4 | 0.0149 |
| Syt13 | NM_030725.4 | 0.0153 |
| Mapk9 | NM_207692.1 | 0.0158 |
| Epha4 | NM_007936.3 | 0.0164 |
| Pla2g16 | NM_139269.2 | 0.0167 |
| Nfkbib | NM_010908.4 | 0.0168 |
| Sod1 | NM_011434.1 | 0.0169 |
| Adam10 | NM_007399.3 | 0.0170 |
| Ptprr | NM_011217.2 | 0.0171 |
| Hif1a | NM_010431.2 | 0.0172 |
| Trim28 | NM_011588.3 | 0.0173 |
| Notch3 | NM_008716.2 | 0.0173 |
| Ring1 | NM_009066.3 | 0.0176 |
| Cnr1 | NM_007726.3 | 0.0177 |
| Atm | NM_009730.2 | 0.0178 |
| Htr1a | NM_008308.4 | 0.0178 |
| Cd14 | NM_009841.3 | 0.0181 |
| Mtor | NM_020009.2 | 0.0182 |
| Comt | NM_007744.3 | 0.0185 |
| Cyp4x1 | NM_001003947.1 | 0.0187 |
| Cx3cr1 | NM_009987.3 | 0.0189 |
| Tnfrsf1a | NM_011609.2 | 0.0190 |
| Hras | NM_001130443.1 | 0.0193 |
| Rit2 | NM_009065.2 | 0.0193 |
| Adra2a | NM_007417.4 | 0.0193 |
| Casp3 | NM_009810.2 | 0.0195 |
| Egr1 | NM_007913.5 | 0.0196 |
| Camk2b | NM_001174053.1 | 0.0196 |
| Taf10 | NM_020024.3 | 0.0200 |
| Aldh1l1 | NM_027406.1 | 0.0202 |
| Adcy8 | NM_009623.2 | 0.0204 |
| Snrpa | NM_001046637.1 | 0.0205 |
| Jun | NM_010591.2 | 0.0207 |
| Tcirg1 | NM_001136091.1 | 0.0215 |
| Sncb | NM_033610.2 | 0.0219 |
| Ang | NM_007447.2 | 0.0220 |
| Usp30 | NM_001033202.3 | 0.0220 |
| Ccs | XM_006531645.1 | 0.0220 |
| Flt4 | NM_008029.3 | 0.0221 |
| Slc2a1 | NM_011400.3 | 0.0223 |
| Palm | NM_023128.4 | 0.0224 |
| Efr3a | NM_133766.3 | 0.0226 |
| Stx1b | NM_024414.2 | 0.0227 |
| Egfr | NM_207655.2 | 0.0228 |
| Sorl1 | NM_011436.3 | 0.0231 |
| Mapk1 | NM_011949.3 | 0.0231 |
| Ccl5 | NM_013653.1 | 0.0232 |
| Cers2 | NM_029789.1 | 0.0233 |
| Aqp4 | NM_009700.2 | 0.0234 |
| Ap2b1 | NM_001035854.2 | 0.0235 |
| Gnao1 | NM_010308.3 | 0.0235 |
| Kcnj10 | NM_001039484.1 | 0.0239 |
| Dnml1 | NM_001025947.1 | 0.0241 |
| Magee1 | NM_053201.3 | 0.0245 |
| Dgke | NM_019505.3 | 0.0249 |
| Fgf12 | NM_001276419.1 | 0.0256 |
| Prkaca | NM_008854.3 | 0.0257 |
| Pcna | NM_011045.2 | 0.0260 |
| Tpm1 | NM_024427.4 | 0.0269 |
| Atcay | NM_178662.3 | 0.0270 |
| Egr2 | NM_010118.2 | 0.0279 |
| Sri | NM_001080974.2 | 0.0280 |
| Pik3ca | NM_008839.1 | 0.0280 |
| Ddx23 | NM_001080981.1 | 0.0281 |
| Cdc40 | NM_027879.2 | 0.0281 |
| Trem2 | NM_031254.2 | 0.0284 |
| Calm1 | NM_009790.4 | 0.0286 |
| Gls | NM_001113383.1 | 0.0287 |
| Cck | NM_031161.2 | 0.0289 |
| Ap2a2 | NM_007459.3 | 0.0290 |
| Adcy9 | NM_009624.1 | 0.0290 |
| Grn | NM_008175.3 | 0.0291 |
| Pdgfrb | NM_008809.1 | 0.0293 |
| Vip | NM_011702.2 | 0.0295 |
| Dagla | NM_198114.2 | 0.0296 |
| Gaa | NM_008064.3 | 0.0301 |
| Cnksr2 | NM_177751.2 | 0.0302 |
| Rasgrp1 | NM_011246.2 | 0.0302 |
| Wfs1 | NM_011716.2 | 0.0303 |
| Epha5 | NM_007937.3 | 0.0303 |
| Htr5a | NM_008314.2 | 0.0310 |
| Hdac7 | NM_019572.2 | 0.0315 |
| Inpp4a | NM_172971.2 | 0.0315 |
| Gsk3b | NM_019827.3 | 0.0316 |
| Mmp14 | NM_008608.3 | 0.0320 |
| Casp6 | NM_009811.3 | 0.0325 |
| Grin1 | NM_008169.2 | 0.0327 |
| Csf1r | NM_001037859.1 | 0.0329 |
| Pink1 | NM_026880.2 | 0.0331 |
| Gria3 | NM_016886.3 | 0.0332 |
| Cntn4 | NM_001109751.1 | 0.0332 |
| Sf3a2 | NM_013651.4 | 0.0334 |
| Racl | NM_009007.2 | 0.0339 |
| Nme5 | NM_080637.3 | 0.0342 |
| Ppp2ca | NM_019411.4 | 0.0347 |
| Car2 | NM_009801.4 | 0.0349 |
| Caenb2 | NM_023116.3 | 0.0350 |
| Hpgds | NM_019455.4 | 0.0352 |
| Map2k2 | NM_023138.4 | 0.0354 |
| Stx1a | NM_016801.3 | 0.0357 |
| Eng | NM_001146350.1 | 0.0360 |
| Lamb2 | NM_008483.3 | 0.0366 |
| Plcb3 | NM_008874.3 | 0.0368 |
| Hmgb1 | NM_010439.3 | 0.0375 |
| Cdk5 | NM_007668.3 | 0.0383 |
| Atxn2 | NM_009125.2 | 0.0397 |
| Tgfb1 | NM_011577.1 | 0.0398 |
| Taf9 | NM_027139.5 | 0.0406 |
| Ape | NM_007462.3 | 0.0407 |
| Ppp3cb | NM_008914.1 | 0.0410 |
| Atp6v1g2 | NM_023179.3 | 0.0412 |
| Scn2a1 | NM_001099298.3 | 0.0412 |
| Gstp1 | NM_013541.1 | 0.0418 |
| Tfam | NM_009360.4 | 0.0419 |
| Pik3r1 | NM_001024955.1 | 0.0420 |
| Cadps | NM_001042617.1 | 0.0425 |
| Plcb1 | NM_019677.1 | 0.0426 |
| Egf17 | NM_001164564.1 | 0.0435 |
| Acvrl1 | NM_009612.2 | 0.0438 |
| Ipcef1 | NM_001033391.2 | 0.0439 |
| Neo1 | NM_001042752.1 | 0.0440 |
| Ap3s1 | NM_009681.5 | 0.0444 |
| Cntnap2 | NM_001004357.2 | 0.0446 |
| I16ra | NM_010559.2 | 0.0452 |
| Opa1 | NM_001199177.1 | 0.0453 |
| Gria4 | NM_001113180.1 | 0.0453 |
| Arhgap44 | NM_001099288.1 | 0.0456 |
| Zfp24 | NM_021559.2 | 0.0461 |
| Cacnb4 | NM_146123.2 | 0.0462 |
| Dnaja2 | NM_019794.4 | 0.0465 |
| Efnal | NM_010107.4 | 0.0468 |
| Chmp2b | NM_026879.2 | 0.0469 |
| Syt7 | NM_018801.3 | 0.0472 |
| Sptbn2 | NM_021287.1 | 0.0474 |
| Epha6 | NM_007938.2 | 0.0477 |
| Bdnf | NM_007540.4 | 0.0478 |
| Smn1 | NM_011420.2 | 0.0481 |
| Tbpl1 | NM_011603.5 | 0.0485 |
| Lrrc4 | NM_138682.2 | 0.0485 |
| Synjl | NM_001045515.1 | 0.0494 |
| Polr2h | NM_145632.2 | 0.0495 |
| Cast | NM_009817.1 | 0.0496 |
| Amigo1 | NM_001004293.2 | 0.0498 |
| Gfpt1 | NM_013528.3 | 0.0498 |

TABLE 6
From TTPB_0400_final document,
Differentially Expressed Genes-DIV03

| Gene Name | Accession # | Pval |
|---|---|---|
| Cxcl12 | NM_021704.3 | 0.0000 |
| Tspo | NM_009775.4 | 0.0000 |
| Sqstm1 | NM_011018.2 | 0.0000 |
| Cd44 | NM_009851.2 | 0.0000 |
| Atp8a2 | NM_015803.2 | 0.0001 |
| Aars | NM_146217.4 | 0.0001 |
| Gjb1 | NM_008124.2 | 0.0001 |
| Epha5 | NM_007937.3 | 0.0001 |
| Gabra4 | NM_010251.2 | 0.0001 |
| Dgkb | NM_178681.4 | 0.0001 |
| Abat | NM_001170978.1 | 0.0001 |
| Gstp1 | NM_013541.1 | 0.0001 |
| Lama2 | NM_008481.2 | 0.0001 |
| Atp2b3 | NM_177236.3 | 0.0001 |
| Myrf | NM_001033481.1 | 0.0001 |
| Cxcr4 | NM_009911.3 | 0.0001 |
| Pfn1 | NM_011072.4 | 0.0001 |
| Mmp12 | NM_008605.3 | 0.0002 |
| Hmoxl | NM_010442.2 | 0.0002 |
| Tmem119 | NM_146162.2 | 0.0002 |
| Clu | NM_013492.2 | 0.0002 |
| Adrb2 | NM_007420.2 | 0.0002 |
| Taz | NM_181516.5 | 0.0002 |
| Plcb1 | NM_019677.1 | 0.0002 |
| Cntn1 | NM_001159647.1 | 0.0002 |
| Src | NM_001025395.2 | 0.0002 |
| Apoe | NM_001305844.1 | 0.0002 |
| Igf1r | NM_010513.2 | 0.0003 |
| Mtor | NM_020009.2 | 0.0003 |
| Negr1 | NM_001039094.2 | 0.0003 |
| Serpinb6a | NM_001164117.1 | 0.0003 |
| Itga5 | NM_001314041.1 | 0.0003 |
| Pmp | NM_011170.2 | 0.0003 |
| Casp7 | NM_007611.2 | 0.0003 |
| Sf3b2 | NM_030109.2 | 0.0003 |
| Taf10 | NM_020024.3 | 0.0003 |
| Gnao1 | NM_010308.3 | 0.0004 |
| Synj1 | NM_001045515.1 | 0.0004 |
| Il10ra | NM_008348.2 | 0.0004 |
| Grin2c | NM_010350.2 | 0.0004 |
| Grm5 | NM_001143834.1 | 0.0004 |
| Xiap | NM_009688.2 | 0.0004 |
| Pnkd | NM_019999.2 | 0.0004 |
| Apaf1 | NM_009684.2 | 0.0004 |
| Insr | NM_010568.2 | 0.0005 |
| Fgf2 | NM_008006.2 | 0.0005 |
| Nrxn1 | NM_001346960.1 | 0.0005 |
| Mag | NM_010758.2 | 0.0005 |
| Cdk5r1 | NM_009871.2 | 0.0005 |
| Ngf | NM_001112698.1 | 0.0006 |
| Grin1 | NM_008169.2 | 0.0006 |
| Hgf | NM_010427.5 | 0.0006 |
| Pla2g4a | NM_008869.2 | 0.0006 |
| Plxnb3 | NM_019587.2 | 0.0007 |
| Tnfrsf12a | NM_001161746.1 | 0.0007 |
| Bax | NM_007527.3 | 0.0007 |
| Lif | NM_008501.2 | 0.0008 |
| Lars | NM_134137.2 | 0.0008 |
| Cdkn1a | NM_007669.4 | 0.0008 |
| Osmr | NM_011019.3 | 0.0009 |
| Gsr | NM_010344.4 | 0.0009 |
| Dlg3 | NM_001177778.1 | 0.0009 |
| Cadm3 | NM_053199.3 | 0.0009 |
| Ddit3 | NM_007837.3 | 0.0010 |
| Slc12a5 | NM_020333.2 | 0.0010 |
| Ccdc127 | NM_024201.3 | 0.0010 |
| AI464131 | NM_001085515.2 | 0.0010 |
| Hdac2 | NM_008229.2 | 0.0010 |
| Col4a2 | NM_009932.3 | 0.0011 |
| Slc1a2 | NM_001077514.3 | 0.0011 |
| Myct1 | NM_026793.2 | 0.0012 |
| Syt1 | NM_009306.2 | 0.0012 |
| Ap4s1 | NM_021710.3 | 0.0012 |
| Itpr1 | NM_010585.2 | 0.0012 |
| Il1r1 | NM_001123382.1 | 0.0012 |
| Entpd2 | NM_009849.2 | 0.0013 |
| Aqp4 | NM_009700.2 | 0.0013 |
| Cd33 | NM_001111058.1 | 0.0013 |
| Egr1 | NM_007913.5 | 0.0013 |
| B4galt6 | NM_019737.2 | 0.0014 |
| Kcnj10 | NM_001039484.1 | 0.0014 |
| Bcas1 | NM_029815.2 | 0.0014 |
| Pcsk2 | NM_008792.3 | 0.0014 |
| Tnc | NM_011607.1 | 0.0014 |
| Gba | NM_001077411.1 | 0.0015 |
| Cx3cl1 | NM_009142.3 | 0.0015 |
| Col4a1 | NM_009931.2 | 0.0015 |
| Hpgds | NM_019455.4 | 0.0015 |
| Cp | NM_001042611.1 | 0.0015 |
| Thy1 | NM_009382.3 | 0.0016 |
| Dagla | NM_198114.2 | 0.0016 |
| Cdc40 | NM_027879.2 | 0.0016 |
| Hif1a | NM_010431.2 | 0.0016 |
| Gnai3 | NM_010306.2 | 0.0016 |
| Atp6v0e | NM_025272.2 | 0.0016 |
| Frmpd4 | NM_001033330.2 | 0.0016 |
| Ncam1 | NM_001113204.1 | 0.0017 |
| Csf1 | NM_001113530.1 | 0.0017 |
| Cd9 | NM_007657.3 | 0.0017 |
| Syt7 | NM_018801.3 | 0.0017 |
| Rab3c | NM_023852.5 | 0.0017 |
| Casp3 | NM_009810.2 | 0.0017 |
| Slc9a6 | NM_172780.3 | 0.0018 |
| Stat3 | NM_213659.2 | 0.0018 |
| Sox9 | NM_011448.4 | 0.0018 |
| Msn | NM_010833.2 | 0.0018 |
| Kif3a | NM_008443.3 | 0.0018 |
| Cd40 | NM_011611.2 | 0.0018 |
| Lrp1 | NM_008512.2 | 0.0018 |
| Htt | NM_010414.1 | 0.0018 |
| Gnptg | NM_172529.3 | 0.0018 |
| Glrb | NM_010298.5 | 0.0018 |
| Grin2b | NM_008171.3 | 0.0019 |
| I14ra | NM_001008700.3 | 0.0019 |
| Tnfrsf10b | NM_020275.3 | 0.0019 |
| Gabrb2 | NM_008070.3 | 0.0020 |
| Pcna | NM_011045.2 | 0.0020 |
| Gria1 | NM_001252403.1 | 0.0021 |
| Magee1 | NM_053201.3 | 0.0021 |
| Ccnd1 | NM_007631.1 | 0.0021 |
| Nell2 | NM_016743.2 | 0.0021 |
| Gabrb3 | NM_008071.3 | 0.0021 |
| Gsn | NM_146120.3 | 0.0021 |
| Angpt2 | NM_007426.3 | 0.0022 |
| Vcp | NM_009503.3 | 0.0022 |
| Jun | NM_010591.2 | 0.0022 |
| Arhgef10 | NM_001037736.1 | 0.0022 |
| P2ry12 | NM_027571.3 | 0.0023 |
| Kcnb1 | NM_008420.3 | 0.0023 |
| Cul1 | NM_012042.3 | 0.0023 |
| Cacnb2 | NM_023116.3 | 0.0023 |
| Sp100 | NM_013673.3 | 0.0023 |
| PlCl2 | NM_013880.3 | 0.0023 |
| Mog | NM_010814.2 | 0.0024 |
| Gtf2h1 | NM_008186.4 | 0.0024 |
| Cd14 | NM_009841.3 | 0.0024 |
| Cacna1b | NM_001042528.1 | 0.0025 |
| Scn1a | NM_018733.2 | 0.0025 |
| Prkce | NM_011104.2 | 0.0025 |
| Sri | NM_001080974.2 | 0.0025 |
| Camk2g | NM_001039138.1 | 0.0025 |
| Atm | NM_009730.2 | 0.0026 |
| Trp53 | NM_011640.1 | 0.0026 |
| Hdac1 | NM_008228.2 | 0.0026 |
| Adra2a | NM_007417.4 | 0.0027 |
| Vegfa | NM_001025250.3 | 0.0027 |
| Nes | NM_016701.3 | 0.0027 |
| I16ra | NM_010559.2 | 0.0027 |
| Slc4a10 | NM_001242380.1 | 0.0028 |
| Gal3st1 | NM_001177691.1 | 0.0028 |
| Csf1r | NM_001037859.1 | 0.0028 |
| Erbb3 | NM_010153.1 | 0.0028 |
| Cycs | NM_007808.4 | 0.0028 |
| Sorll | NM_011436.3 | 0.0029 |
| Efnal | NM_010107.4 | 0.0029 |
| Dlat | NM_145614.4 | 0.0030 |
| Efna5 | NM_207654.2 | 0.0030 |
| Polr2h | NM_145632.2 | 0.0031 |
| Atf4 | NM_009716.2 | 0.0031 |
| Gria4 | NM_001113180.1 | 0.0033 |
| Ring1 | NM_009066.3 | 0.0034 |
| Gprasp1 | NM_001004359.2 | 0.0034 |
| Gfap | NM_001131020.1 | 0.0034 |
| Gnb5 | NM_138719.5 | 0.0034 |
| Fcrls | NM_030707.3 | 0.0035 |
| Mapk10 | NM_001081567.1 | 0.0035 |
| Flt1 | NM_010228.3 | 0.0035 |
| Rapgef2 | NM_001099624.2 | 0.0035 |
| Nova1 | NM_021361.1 | 0.0036 |
| Cd68 | NM_009853.1 | 0.0037 |
| Snca | NM_009221.2 | 0.0037 |
| C3 | XM_011246258.1 | 0.0037 |
| Pla2g4b | XM_925095.2 | 0.0037 |
| Dnml1 | NM_001025947.1 | 0.0038 |
| Car2 | NM_009801.4 | 0.0038 |
| Ryr2 | NM_023868.1 | 0.0039 |
| Prkacb | NM_011100.3 | 0.0039 |
| Fmr1 | NM_008031.2 | 0.0040 |
| Tie1 | NM_011587.2 | 0.0041 |
| Trpm2 | NM_138301.2 | 0.0041 |
| Nef1 | NM_010910.1 | 0.0043 |
| Comt | NM_007744.3 | 0.0043 |
| Sirt2 | NM_022432.4 | 0.0043 |
| Gucy1b3 | NM_017469.4 | 0.0045 |
| Gabra1 | NM_010250.4 | 0.0046 |
| Map2k1 | NM_008927.3 | 0.0046 |
| Mbp | NM_010777.3 | 0.0046 |
| Cx3cr1 | NM_009987.3 | 0.0047 |
| Ptprn2 | NM_011215.2 | 0.0047 |
| App | NM_007471.2 | 0.0049 |
| Cnot10 | NM_153585.5 | 0.0049 |
| Jam3 | NM_023277.4 | 0.0049 |
| Mal | NM_001171187.1 | 0.0049 |
| Slc2a1 | NM_011400.3 | 0.0049 |
| Optn | NM_181848.4 | 0.0050 |
| Tbr1 | NM_009322.3 | 0.0051 |
| Npy | NM_023456.2 | 0.0051 |
| Gria3 | NM_016886.3 | 0.0052 |
| Camk4 | NM_009793.3 | 0.0052 |
| Cast | NM_009817.1 | 0.0052 |
| Trf | NM_133977.2 | 0.0053 |
| Tenm2 | NM_011856.3 | 0.0054 |
| Ltbr | NM_010736.3 | 0.0054 |
| Ube2n | NM_080560.3 | 0.0055 |
| Gpdl1 | NM_175380.5 | 0.0055 |
| Polr2k | NM_001039368.1 | 0.0055 |
| Ptgs2 | NM_011198.3 | 0.0055 |
| Islr2 | NM_001161538.1 | 0.0055 |
| Cnksr2 | NM_177751.2 | 0.0056 |
| Grm2 | NM_001160353.1 | 0.0056 |
| Cers2 | NM_029789.1 | 0.0057 |
| Igf1 | NM_010512.4 | 0.0057 |
| Gtf2h3 | NM_181410.3 | 0.0058 |
| Cds1 | NM_173370.3 | 0.0059 |
| Tnfrsf1a | NM_011609.2 | 0.0059 |
| Eif2s1 | NM_026114.3 | 0.0059 |
| Sod2 | NM_013671.3 | 0.0059 |
| Cln8 | NM_012000.3 | 0.0060 |
| Cck | NM_031161.2 | 0.0060 |
| Olig2 | NM_016967.2 | 0.0060 |
| Rab3a | NM_009001.6 | 0.0063 |
| Mfn2 | XM_006535920.1 | 0.0063 |
| Ipcef1 | NM_001033391.2 | 0.0063 |
| Ache | NM_009599.3 | 0.0064 |
| Dlg4 | NM_001109752.1 | 0.0065 |
| P2rx4 | NM_011026.2 | 0.0065 |
| Nptn | NM_009145.2 | 0.0065 |
| Mmp14 | NM_008608.3 | 0.0066 |
| Lmna | NM_001002011.2 | 0.0067 |
| Ncf1 | NM_001286037.1 | 0.0067 |
| Map2 | NM_008632.2 | 0.0072 |
| Syt13 | NM_030725.4 | 0.0073 |
| Sncb | NM_033610.2 | 0.0073 |
| Lcla1l | NM_001177967.1 | 0.0073 |
| Htr1a | NM_008308.4 | 0.0074 |
| Pecaml | NM_008816.2 | 0.0077 |
| Grml | NM_001114333.2 | 0.0077 |
| Atcay | NM_178662.3 | 0.0078 |
| Scn2a1 | NM_001099298.3 | 0.0078 |
| Lsm2 | NM_001110101.2 | 0.0079 |
| Nosl | NM_008712.2 | 0.0084 |
| Unc13a | NM_001029873.2 | 0.0086 |
| Cntnap1 | NM_016782.2 | 0.0086 |
| Stambpl1 | NM_029682.4 | 0.0088 |
| Idh1 | NM_010497.2 | 0.0088 |
| Slclla1 | NM_013612.2 | 0.0091 |
| Fyn | NM_008054.2 | 0.0092 |
| Ppp2r5e | NM_012024.2 | 0.0095 |
| Ptdss2 | NM_013782.4 | 0.0096 |
| Cntnap2 | NM_001004357.2 | 0.0096 |
| Ppp3ca | NM_008913.4 | 0.0096 |
| Ggt1 | NM_008116.2 | 0.0097 |
| Gria2 | NM_001039195.1 | 0.0097 |
| Mmm2 | NM_153127.3 | 0.0098 |
| Erlec1 | NM_025745.3 | 0.0099 |
| Cxcl16 | NM_023158.6 | 0.0099 |
| Fn1 | NM_010233.1 | 0.0100 |
| Ep300 | NM_177821.6 | 0.0102 |
| Emcn | NM_001163522.1 | 0.0104 |
| Aktlsl | NM_026270.4 | 0.0104 |
| Epha4 | NM_007936.3 | 0.0107 |
| Tardbp | NM_001003899.2 | 0.0108 |
| Cntn4 | NM_001109751.1 | 0.0108 |
| Ppargc1a | NM_008904.2 | 0.0108 |
| Gnai2 | NM_008138.4 | 0.0109 |
| Gfpt1 | NM_013528.3 | 0.0113 |
| Hexb | NM_010422.2 | 0.0114 |
| Ccnh | NM_023243.2 | 0.0115 |
| Flt4 | NM_008029.3 | 0.0116 |
| Nlrp3 | NM_145827.3 | 0.0117 |
| Nsf | NM_008740.2 | 0.0119 |
| Crebbp | NM_001025432.1 | 0.0119 |
| Cacna1a | NM_007578.3 | 0.0119 |
| Adcy8 | NM_009623.2 | 0.0119 |
| Snrpa | NM_001046637.1 | 0.0121 |
| Slc17a6 | NM_080853.3 | 0.0121 |
| Prkcb | NM_008855.2 | 0.0122 |
| Fos | NM_010234.2 | 0.0123 |
| Grin2a | NM_008170.2 | 0.0125 |
| Tbpl1 | NM_011603.5 | 0.0125 |
| Xbp1 | NM_013842.2 | 0.0127 |
| Myc | NM_010849.4 | 0.0127 |
| Camk2b | NM_001174053.1 | 0.0128 |
| Ube2k | NM_016786.3 | 0.0129 |
| Itpr2 | NM_010586.1 | 0.0130 |
| Suc1a2 | NM_011506.1 | 0.0132 |
| L1cam | NM_008478.3 | 0.0133 |
| Calb2 | NM_007586.1 | 0.0133 |
| Icam2 | NM_010494.1 | 0.0134 |
| Nefh | NM_010904.3 | 0.0137 |
| Ugt8a | NM_011674.4 | 0.0139 |
| Stab1 | NM_138672.2 | 0.0139 |
| Ap3m2 | NM_029505.3 | 0.0140 |
| Akt2 | NM_001110208.1 | 0.0141 |
| Ptdss1 | NM_008959.3 | 0.0141 |
| Cadps | NM_001042617.1 | 0.0144 |
| Epha7 | NM_001122889.1 | 0.0145 |
| Pik3ca | NM_008839.1 | 0.0146 |
| Ptprr | NM_011217.2 | 0.0149 |
| Gabrg2 | NM_177408.5 | 0.0149 |
| Ddc | NM_016672.4 | 0.0149 |
| Gls | NM_001113383.1 | 0.0149 |
| Slc8a1 | NM_011406.2 | 0.0150 |
| Mapk3 | NM_011952.2 | 0.0150 |
| Actn1 | NM_134156.2 | 0.0153 |
| Vip | NM_011702.2 | 0.0161 |
| Rit2 | NM_009065.2 | 0.0162 |
| Tgfb1 | NM_011577.1 | 0.0163 |
| Mapk8 | NM_016700.3 | 0.0166 |
| Gata2 | NM_008090.4 | 0.0168 |
| Fam104a | NM_138598.5 | 0.0168 |
| Akt1 | NM_001165894.1 | 0.0171 |
| Sgpl1 | NM_009163.3 | 0.0172 |
| Gsk3b | NM_019827.3 | 0.0172 |
| Man2b1 | NM_010764.2 | 0.0172 |
| Notch3 | NM_008716.2 | 0.0173 |
| Park2 | NM_016694.3 | 0.0175 |
| Mapk1 | NM_011949.3 | 0.0176 |
| Atp6v0e2 | NM_133764.3 | 0.0176 |
| Dgke | NM_019505.3 | 0.0178 |
| Chrna7 | NM_007390.3 | 0.0180 |
| Grik2 | NM_010349.2 | 0.0181 |
| Calb1 | NM_009788.4 | 0.0183 |
| Dll4 | NM_019454.2 | 0.0186 |
| Nrgl | NM_178591.2 | 0.0191 |
| Slclal | NM_009199.2 | 0.0194 |
| Atxn2 | NM_009125.2 | 0.0196 |
| Trim37 | NM_197987.2 | 0.0196 |
| Fgf12 | NM_001276419.1 | 0.0200 |
| Fgf14 | NM_207667.3 | 0.0202 |
| Pak1 | NM_011035.2 | 0.0202 |
| Aldh1l1 | NM_027406.1 | 0.0203 |
| Akt3 | NM_011785.3 | 0.0205 |
| Rab2a | NM_021518.3 | 0.0205 |
| Gaa | NM_008064.3 | 0.0207 |
| Adcy9 | NM_009624.1 | 0.0211 |
| Atp13a2 | NM_029097.2 | 0.0213 |
| Clqa | NM_007572.2 | 0.0217 |
| Ang | NM_007447.2 | 0.0220 |
| Myh10 | NM_175260.2 | 0.0222 |
| Sorcs3 | NM_025696.3 | 0.0222 |
| Clqc | NM_007574.2 | 0.0223 |
| Cacna1d | NM_028981.2 | 0.0226 |
| I16 | NM_031168.1 | 0.0232 |
| Gpr37 | NM_010338.2 | 0.0234 |
| Emp2 | NM_007929.2 | 0.0234 |
| Nmnat2 | NM_175460.3 | 0.0237 |
| Wfs1 | NM_011716.2 | 0.0238 |
| Nf1 | NM_010897.2 | 0.0240 |
| Psen1 | NM_008943.2 | 0.0241 |
| Cyp4x1 | NM_001003947.1 | 0.0249 |
| Gnaq | NM_008139.5 | 0.0249 |
| Chl1 | NM_007697.2 | 0.0257 |
| Mapk9 | NM_207692.1 | 0.0258 |
| Pde1b | NM_008800.1 | 0.0259 |
| C1qb | NM_009777.2 | 0.0260 |
| Stx1a | NM_016801.3 | 0.0261 |
| Bid | NM_007544.3 | 0.0267 |
| Ppml1 | NM_178726.3 | 0.0269 |
| Inpp4a | NM_172971.2 | 0.0270 |
| Rasgrp1 | NM_011246.2 | 0.0273 |
| Cnr1 | NM_007726.3 | 0.0275 |
| Shank2 | NM_001081370.2 | 0.0277 |
| Ap2b1 | NM_001035854.2 | 0.0282 |
| Prkcg | NM_011102.3 | 0.0282 |
| Lsm7 | NM_025349.2 | 0.0284 |
| Atp6v1g2 | NM_023179.3 | 0.0285 |
| Arrb2 | NM_145429.4 | 0.0286 |
| Nostrin | NM_181547.3 | 0.0287 |
| Trim28 | NM_011588.3 | 0.0292 |
| Ccr5 | NM_009917.5 | 0.0292 |
| Mapkapk2 | NM_008551.1 | 0.0294 |
| Stx1b | NM_024414.2 | 0.0295 |
| Nfe2l2 | NR_132727.1 | 0.0296 |
| Tnfrsf1b | NM_011610.3 | 0.0297 |
| Ran | NM_009391.3 | 0.0299 |
| Drd1 | NM_010076.3 | 0.0302 |
| Ppp2ca | NM_019411.4 | 0.0302 |
| Hcn1 | NM_010408.3 | 0.0303 |
| Gng2 | NM_010315.4 | 0.0304 |
| Htr5a | NM_008314.2 | 0.0310 |
| Ppp3cc | NM_008915.2 | 0.0316 |
| Ap2a2 | NM_007459.3 | 0.0320 |
| Hspb1 | NM_013560.2 | 0.0322 |
| Atxn7 | NM_139227.4 | 0.0322 |
| Egr2 | NM_010118.2 | 0.0324 |
| Cxxc1 | NM_028868.3 | 0.0327 |
| Pla2g16 | NM_139269.2 | 0.0327 |
| Itgam | NM_008401.2 | 0.0328 |
| Cers1 | NM_138647.3 | 0.0329 |
| Sf3a2 | NM_013651.4 | 0.0329 |
| Ppp3cb | NM_008914.1 | 0.0330 |
| Gusb | NM_010368.1 | 0.0334 |
| Dlgap1 | NM_027712.3 | 0.0336 |
| Tnr | NM_022312.3 | 0.0338 |
| Nts | NM_024435.2 | 0.0339 |
| Tcirg1 | NM_001136091.1 | 0.0354 |
| Taf4 | NM_001081092.1 | 0.0355 |
| Olfm3 | NM_153157.3 | 0.0363 |
| Bdnf | NM_007540.4 | 0.0367 |
| Nos3 | NM_008713.4 | 0.0368 |
| Entpd4 | NM_026174.2 | 0.0370 |
| Epha6 | NM_007938.2 | 0.0370 |
| Nqo1 | NM_008706.5 | 0.0372 |
| Sptbn2 | NM_021287.1 | 0.0373 |
| Efr3a | NM_133766.3 | 0.0374 |
| Naglu | NM_013792.2 | 0.0377 |
| Smpd4 | NM_001164610.1 | 0.0379 |
| Brmsl1 | NM_001037756.2 | 0.0392 |
| Snap91 | NM_001277986.1 | 0.0393 |
| Pdpk1 | NM_001080773.2 | 0.0394 |
| Amigo1 | NM_001004293.2 | 0.0395 |
| Egf17 | NM_001164564.1 | 0.0395 |
| Gtf2a1 | NM_175335.3 | 0.0400 |
| Slu7 | NM_148673.3 | 0.0410 |
| Esam | NM_027102.3 | 0.0410 |
| Ina | NM_146100.4 | 0.0427 |
| Arhgap44 | NM_001099288.1 | 0.0436 |
| Tlr4 | NM_021297.2 | 0.0444 |
| Amph | NM_175007.1 | 0.0445 |
| Nr4a2 | NM_001139509.1 | 0.0446 |
| Calm1 | NM_009790.4 | 0.0449 |
| Mecp2 | NM_010788.2 | 0.0451 |
| Sox10 | XM_128139.6 | 0.0452 |
| Sec23a | NM_009147.2 | 0.0453 |
| Lsr | NM_001164184.1 | 0.0461 |
| Nwd1 | NM_176940.5 | 0.0463 |
| Rad23b | NM_009011.4 | 0.0467 |
| Bcas2 | NM_026602.3 | 0.0469 |
| Bnip3 | NM_009760.4 | 0.0470 |
| Ryr1 | NM_009109.1 | 0.0478 |
| Syt4 | NM_009308.3 | 0.0478 |
| Homer1 | NM_147176.2 | 0.0478 |
| Lrrk2 | NM_025730.3 | 0.0487 |
| S100b | NM_009115.3 | 0.0487 |
| Oxr1 | NM_001130163.1 | 0.0492 |
| Irf8 | NM_008320.3 | 0.0500 |

TABLE 7
From TTPB_0400_final document,
Differentially Expressed Genes-DIV04

| Gene Name | Accession # | Pval |
|---|---|---|
| Hmox1 | NM_010442.2 | 0.0000 |
| Pla2g4a | NM_008869.2 | 0.0000 |
| Clu | NM_013492.2 | 0.0001 |
| Cd44 | NM_009851.2 | 0.0001 |
| Cx3cr1 | NM_009987.3 | 0.0001 |
| Lama2 | NM_008481.2 | 0.0001 |
| Apoe | NM_001305844.1 | 0.0001 |
| Wfs1 | NM_011716.2 | 0.0001 |
| Adrb2 | NM_007420.2 | 0.0001 |
| Pnkd | NM_019999.2 | 0.0001 |
| Pfn1 | NM_011072.4 | 0.0001 |
| Pmp | NM_011170.2 | 0.0001 |
| Cxcr4 | NM_009911.3 | 0.0002 |
| Cnr1 | NM_007726.3 | 0.0002 |
| Cycs | NM_007808.4 | 0.0002 |
| Grm5 | NM_001143834.1 | 0.0002 |
| Ap4s1 | NM_021710.3 | 0.0002 |
| Synj1 | NM_001045515.1 | 0.0002 |
| Epha5 | NM_007937.3 | 0.0002 |
| Cdk5r1 | NM_009871.2 | 0.0003 |
| Sqstm1 | NM_011018.2 | 0.0003 |
| Slc12a5 | NM_020333.2 | 0.0003 |
| Plcb1 | NM_019677.1 | 0.0003 |
| Atp2b3 | NM_177236.3 | 0.0004 |
| Mtor | NM_020009.2 | 0.0004 |
| Syt1 | NM_009306.2 | 0.0004 |
| Camk2g | NM_001039138.1 | 0.0004 |
| Sorl1 | NM_011436.3 | 0.0004 |
| Slc2a1 | NM_011400.3 | 0.0004 |
| Grm8 | NM_008174.2 | 0.0005 |
| Itga5 | NM_001314041.1 | 0.0005 |
| Cdkn1a | NM_007669.4 | 0.0005 |
| Mmm2 | NM_153127.3 | 0.0006 |
| Nrxn1 | NM_001346960.1 | 0.0007 |
| Ngf | NM_001112698.1 | 0.0007 |
| Adra2a | NM_007417.4 | 0.0007 |
| Gprasp1 | NM_001004359.2 | 0.0007 |
| Cds1 | NM_173370.3 | 0.0007 |
| Csf1 | NM_001113530.1 | 0.0007 |
| Negr1 | NM_001039094.2 | 0.0007 |
| Gabra1 | NM_010250.4 | 0.0007 |
| Tmem119 | NM_146162.2 | 0.0008 |
| Cp | NM_001042611.1 | 0.0008 |
| Lmna | NM_001002011.2 | 0.0008 |
| Trpm2 | NM_138301.2 | 0.0008 |
| Epha7 | NM_001122889.1 | 0.0009 |
| Gabra4 | NM_010251.2 | 0.0009 |
| Map2k1 | NM_008927.3 | 0.0009 |
| Dgkb | NM_178681.4 | 0.0009 |
| Gnb5 | NM_138719.5 | 0.0009 |
| Slc4a10 | NM_001242380.1 | 0.0010 |
| Grin2b | NM_008171.3 | 0.0011 |
| Gba | NM_001077411.1 | 0.0011 |
| Bcasl | NM_029815.2 | 0.0011 |
| Dagla | NM_198114.2 | 0.0012 |
| Il10ra | NM_008348.2 | 0.0012 |
| Scnla | NM_018733.2 | 0.0012 |
| Pcsk2 | NM_008792.3 | 0.0012 |
| Cd9 | NM_007657.3 | 0.0012 |
| Casp3 | NM_009810.2 | 0.0012 |
| Htt | NM_010414.1 | 0.0013 |
| Gria1 | NM_001252403.1 | 0.0013 |
| B4galt6 | NM_019737.2 | 0.0013 |
| Igf1r | NM_010513.2 | 0.0014 |
| PlCl2 | NM_013880.3 | 0.0014 |
| Gabrb3 | NM_008071.3 | 0.0014 |
| Csf1r | NM_001037859.1 | 0.0014 |
| Gjb1 | NM_008124.2 | 0.0014 |
| Park2 | NM_016694.3 | 0.0015 |
| Rasgrp1 | NM_011246.2 | 0.0015 |
| Mbp | NM_010777.3 | 0.0015 |
| Mmp12 | NM_008605.3 | 0.0015 |
| Stat3 | NM_213659.2 | 0.0016 |
| Cntn1 | NM_001159647.1 | 0.0016 |
| Cast | NM_009817.1 | 0.0016 |
| Gsr | NM_010344.4 | 0.0016 |
| Serpinb6a | NM_001164117.1 | 0.0017 |
| Lsr | NM_001164184.1 | 0.0017 |
| Frmpd4 | NM_001033330.2 | 0.0018 |
| Mag | NM_010758.2 | 0.0018 |
| Fgf14 | NM_207667.3 | 0.0018 |
| Glrb | NM_010298.5 | 0.0018 |
| Cxcl12 | NM_021704.3 | 0.0019 |
| Pecam1 | NM_008816.2 | 0.0019 |
| Gnao1 | NM_010308.3 | 0.0019 |
| Tnc | NM_011607.1 | 0.0019 |
| Ddit3 | NM_007837.3 | 0.0019 |
| Atp8a2 | NM_015803.2 | 0.0019 |
| Ptgs2 | NM_011198.3 | 0.0020 |
| Vegfa | NM_001025250.3 | 0.0020 |
| Hgf | NM_010427.5 | 0.0020 |
| P2ry12 | NM_027571.3 | 0.0021 |
| Crebbp | NM_001025432.1 | 0.0022 |
| Myct1 | NM_026793.2 | 0.0022 |
| Itpr1 | NM_010585.2 | 0.0022 |
| Osmr | NM_011019.3 | 0.0023 |
| Prkcg | NM_011102.3 | 0.0023 |
| Grin2c | NM_010350.2 | 0.0023 |
| Sp100 | NM_013673.3 | 0.0023 |
| Gabrb2 | NM_008070.3 | 0.0023 |
| Col4a2 | NM_009932.3 | 0.0024 |
| Dlg3 | NM_001177778.1 | 0.0024 |
| Nos1 | NM_008712.2 | 0.0024 |
| Angpt2 | NM_007426.3 | 0.0024 |
| Ptprn2 | NM_011215.2 | 0.0024 |
| Cd68 | NM_009853.1 | 0.0025 |
| Cadm3 | NM_053199.3 | 0.0026 |
| Flt1 | NM_010228.3 | 0.0026 |
| Calb1 | NM_009788.4 | 0.0026 |
| Tspo | NM_009775.4 | 0.0027 |
| Col4a1 | NM_009931.2 | 0.0027 |
| Olfm3 | NM_153157.3 | 0.0028 |
| Kifa | NM_008443.3 | 0.0028 |
| Chl1 | NM_007697.2 | 0.0028 |
| Cers1 | NM_138647.3 | 0.0029 |
| Gstp1 | NM_013541.1 | 0.0029 |
| Tada2b | NM_001170454.1 | 0.0029 |
| Thy1 | NM_009382.3 | 0.0030 |
| Cx3cl1 | NM_009142.3 | 0.0030 |
| Grm1 | NM_001114333.2 | 0.0030 |
| Gnptg | NM_172529.3 | 0.0030 |
| Gfap | NM_001131020.1 | 0.0030 |
| AI464131 | NM_001085515.2 | 0.0031 |
| Gaa | NM_008064.3 | 0.0031 |
| C3 | XM_011246258.1 | 0.0031 |
| Epha4 | NM_007936.3 | 0.0032 |
| Cxc116 | NM_023158.6 | 0.0032 |
| Ryr2 | NM_023868.1 | 0.0032 |
| Tnfrsf12a | NM_001161746.1 | 0.0032 |
| Unc13a | NM_001029873.2 | 0.0033 |
| Cd14 | NM_009841.3 | 0.0034 |
| Gria3 | NM_016886.3 | 0.0034 |
| Dlat | NM_145614.4 | 0.0034 |
| Lamb2 | NM_008483.3 | 0.0035 |
| Grin1 | NM_008169.2 | 0.0035 |
| Cacna1b | NM_001042528.1 | 0.0037 |
| Pla2g4b | XM_925095.2 | 0.0037 |
| Cacnb2 | NM_023116.3 | 0.0038 |
| Car2 | NM_009801.4 | 0.0039 |
| Trp53 | NM_011640.1 | 0.0040 |
| Myc | NM_010849.4 | 0.0040 |
| Aqp4 | NM_009700.2 | 0.0040 |
| Rapgef2 | NM_001099624.2 | 0.0042 |
| Msn | NM_010833.2 | 0.0043 |
| Fn1 | NM_010233.1 | 0.0043 |
| Jun | NM_010591.2 | 0.0043 |
| I14ra | NM_001008700.3 | 0.0043 |
| Magee1 | NM_053201.3 | 0.0044 |
| Nf1 | NM_010897.2 | 0.0044 |
| Cntn4 | NM_001109751.1 | 0.0045 |
| Gucy1b3 | NM_017469.4 | 0.0045 |
| Scn2a1 | NM_001099298.3 | 0.0046 |
| Camk2b | NM_001174053.1 | 0.0046 |
| Cers2 | NM_029789.1 | 0.0047 |
| Abat | NM_001170978.1 | 0.0048 |
| Tnfrsf1a | NM_011609.2 | 0.0049 |
| P2rx4 | NM_011026.2 | 0.0049 |
| Nell2 | NM_016743.2 | 0.0050 |
| Snca | NM_009221.2 | 0.0050 |
| Trf | NM_133977.2 | 0.0051 |
| Egr1 | NM_007913.5 | 0.0051 |
| Camk4 | NM_009793.3 | 0.0052 |
| Ppargc1a | NM_008904.2 | 0.0052 |
| Slc11a1 | NM_013612.2 | 0.0052 |
| Eif2s1 | NM_026114.3 | 0.0053 |
| Taz | NM_181516.5 | 0.0054 |
| Hspb1 | NM_013560.2 | 0.0055 |
| Gria2 | NM_001039195.1 | 0.0055 |
| Myrf | NM_001033481.1 | 0.0057 |
| Smn1 | NM_011420.2 | 0.0057 |
| Slc1a2 | NM_001077514.3 | 0.0058 |
| Gls | NM_001113383.1 | 0.0061 |
| Tnfrsf10b | NM_020275.3 | 0.0062 |
| Rab3c | NM_023852.5 | 0.0063 |
| Lif | NM_008501.2 | 0.0063 |
| Syt7 | NM_018801.3 | 0.0064 |
| Ache | NM_009599.3 | 0.0067 |
| Slc8a1 | NM_011406.2 | 0.0068 |
| Ddc | NM_016672.4 | 0.0072 |
| Nptn | NM_009145.2 | 0.0072 |
| Adcy8 | NM_009623.2 | 0.0073 |
| Tnfrsf11b | NM_008764.3 | 0.0074 |
| Prkacb | NM_011100.3 | 0.0074 |
| Fcrls | NM_030707.3 | 0.0075 |
| Prkce | NM_011104.2 | 0.0075 |
| Mapk10 | NM_001081567.1 | 0.0075 |
| Hdacl | NM_008228.2 | 0.0075 |
| Rab3a | NM_009001.6 | 0.0076 |
| Atcay | NM_178662.3 | 0.0076 |
| Sri | NM_001080974.2 | 0.0076 |
| Mfn2 | XM_006535920.1 | 0.0076 |
| Dgke | NM_019505.3 | 0.0078 |
| Ccnd1 | NM_007631.1 | 0.0078 |
| Cck | NM_031161.2 | 0.0078 |
| Sorcs3 | NM_025696.3 | 0.0081 |
| Cd40 | NM_011611.2 | 0.0084 |
| Nqo1 | NM_008706.5 | 0.0084 |
| Casp7 | NM_007611.2 | 0.0086 |
| Sgpl1 | NM_009163.3 | 0.0089 |
| Hcn1 | NM_010408.3 | 0.0090 |
| Csf2rb | NM_007780.4 | 0.0090 |
| Ube2n | NM_080560.3 | 0.0091 |
| Cd33 | NM_001111058.1 | 0.0092 |
| Drd1 | NM_010076.3 | 0.0092 |
| Grik2 | NM_010349.2 | 0.0092 |
| Ring1 | NM_009066.3 | 0.0093 |
| Rit2 | NM_009065.2 | 0.0093 |
| Nlrp3 | NM_145827.3 | 0.0094 |
| Lrrk2 | NM_025730.3 | 0.0094 |
| Ggt1 | NM_008116.2 | 0.0097 |
| Syt13 | NM_030725.4 | 0.0100 |
| Lrp1 | NM_008512.2 | 0.0100 |
| Dnml1 | NM_001025947.1 | 0.0102 |
| Vip | NM_011702.2 | 0.0104 |
| Htr1a | NM_008308.4 | 0.0107 |
| Psen2 | NM_001128605.1 | 0.0109 |
| Entpd2 | NM_009849.2 | 0.0110 |
| Cacna1a | NM_007578.3 | 0.0111 |
| Gsk3b | NM_019827.3 | 0.0114 |
| Grm2 | NM_001160353.1 | 0.0115 |
| Efna5 | NM_207654.2 | 0.0115 |
| Sncb | NM_033610.2 | 0.0117 |
| Icam2 | NM_010494.1 | 0.0119 |
| lllr1 | NM_001123382.1 | 0.0120 |
| Apaf1 | NM_009684.2 | 0.0120 |
| Sucla2 | NM_011506.1 | 0.0120 |
| Nes | NM_016701.3 | 0.0120 |
| Polr2k | NM_001039368.1 | 0.0123 |
| Cnksr2 | NM_177751.2 | 0.0125 |
| Bax | NM_007527.3 | 0.0125 |
| Grin2a | NM_008170.2 | 0.0125 |
| Ang | NM_007447.2 | 0.0128 |
| Slc9a6 | NM_172780.3 | 0.0129 |
| Atp13a2 | NM_029097.2 | 0.0130 |
| Atp6v0e2 | NM_133764.3 | 0.0130 |
| Atf4 | NM_009716.2 | 0.0130 |
| Fgf2 | NM_008006.2 | 0.0131 |
| Tbpl1 | NM_011603.5 | 0.0131 |
| Mog | NM_010814.2 | 0.0134 |
| Nrg1 | NM_178591.2 | 0.0134 |
| Kcnb1 | NM_008420.3 | 0.0135 |
| Prkcb | NM_008855.2 | 0.0136 |
| Adcy9 | NM_009624.1 | 0.0137 |
| Pla2g6 | NM_001199023.1 | 0.0137 |
| Npy | NM_023456.2 | 0.0139 |
| Fos | NM_010234.2 | 0.0140 |
| Notch3 | NM_008716.2 | 0.0140 |
| Emp2 | NM_007929.2 | 0.0141 |
| lgf1 | NM_010512.4 | 0.0141 |
| Prkaa2 | NM_178143.1 | 0.0142 |
| Nsf | NM_008740.2 | 0.0144 |
| Cntnap1 | NM_016782.2 | 0.0146 |
| Gpdl1 | NM_175380.5 | 0.0147 |
| Sox9 | NM_011448.4 | 0.0147 |
| Tgfb1 | NM_011577.1 | 0.0150 |
| Cln8 | NM_012000.3 | 0.0151 |
| Aidhill | NM_027406.1 | 0.0153 |
| Fgf12 | NM_001276419.1 | 0.0154 |
| Nostrin | NM_181547.3 | 0.0155 |
| Stabl | NM_138672.2 | 0.0156 |
| Tiel | NM_011587.2 | 0.0157 |
| Pakl | NM_011035.2 | 0.0158 |
| lnpp4a | NM_172971.2 | 0.0162 |
| Tenm2 | NM_011856.3 | 0.0165 |
| Ppp2r5e | NM_012024.2 | 0.0167 |
| Amigo1 | NM_001004293.2 | 0.0167 |
| Optn | NM_181848.4 | 0.0169 |
| Ppp3ca | NM_008913.4 | 0.0170 |
| Mmpl4 | NM_008608.3 | 0.0170 |
| Plxnb3 | NM_019587.2 | 0.0173 |
| Xiap | NM_009688.2 | 0.0175 |
| Nr4a2 | NM_001139509.1 | 0.0176 |
| Cadps | NM_001042617.1 | 0.0180 |
| I16ra | NM_010559.2 | 0.0181 |
| Cdc40 | NM_027879.2 | 0.0181 |
| Ap3m2 | NM_029505.3 | 0.0183 |
| Atg5 | NM_053069.5 | 0.0185 |
| Man2bl | NM_010764.2 | 0.0187 |
| Gabrg2 | NM_177408.5 | 0.0191 |
| Mapk3 | NM_011952.2 | 0.0191 |
| Trim37 | NM_197987.2 | 0.0194 |
| Psenl | NM_008943.2 | 0.0195 |
| Hifla | NM_010431.2 | 0.0196 |
| Egfr | NM_207655.2 | 0.0196 |
| Gal3stl | NM_001177691.1 | 0.0199 |
| Gria4 | NM_001113180.1 | 0.0199 |
| Ina | NM_146100.4 | 0.0200 |
| Gsn | NM_146120.3 | 0.0202 |
| Tardbp | NM_001003899.2 | 0.0204 |
| Ncam1 | NM_001113204.1 | 0.0207 |
| Cul1 | NM_012042.3 | 0.0210 |
| Hexb | NM_010422.2 | 0.0211 |
| Prkaca | NM_008854.3 | 0.0213 |
| Pla2g16 | NM_139269.2 | 0.0213 |
| Map2 | NM_008632.2 | 0.0218 |
| Cacna1d | NM_028981.2 | 0.0219 |
| Arhgef10 | NM_001037736.1 | 0.0221 |
| Vcp | NM_009503.3 | 0.0224 |
| Atm | NM_009730.2 | 0.0226 |
| Fmr1 | NM_008031.2 | 0.0226 |
| Ran | NM_009391.3 | 0.0227 |
| Naglu | NM_013792.2 | 0.0229 |
| Ppp3cb | NM_008914.1 | 0.0231 |
| Tnfrsf1b | NM_011610.3 | 0.0232 |
| Src | NM_001025395.2 | 0.0232 |
| Epha6 | NM_007938.2 | 0.0232 |
| Chrna7 | NM_007390.3 | 0.0232 |
| Stambpl1 | NM_029682.4 | 0.0233 |
| Atxn2 | NM_009125.2 | 0.0235 |
| Myh10 | NM_175260.2 | 0.0235 |
| Idh1 | NM_010497.2 | 0.0236 |
| Plcb4 | NM_013829.2 | 0.0238 |
| Mapk1 | NM_011949.3 | 0.0247 |
| Cyp4x1 | NM_001003947.1 | 0.0249 |
| Mapk8 | NM_016700.3 | 0.0257 |
| Gdpd2 | NM_023608.3 | 0.0262 |
| Ncf1 | NM_001286037.1 | 0.0268 |
| Lrrc4 | NM_138682.2 | 0.0269 |
| Comt | NM_007744.3 | 0.0271 |
| Ube2k | NM_016786.3 | 0.0271 |
| App | NM_007471.2 | 0.0273 |
| Nwd1 | NM_176940.5 | 0.0279 |
| Efr3a | NM_133766.3 | 0.0281 |
| Kcnj10 | NM_001039484.1 | 0.0282 |
| Nfe2l2 | NR_132727.1 | 0.0284 |
| C1qb | NM_009777.2 | 0.0284 |
| Ipcef1 | NM_001033391.2 | 0.0288 |
| Taf10 | NM_020024.3 | 0.0290 |
| Tcirg1 | NM_001136091.1 | 0.0291 |
| Erbb3 | NM_010153.1 | 0.0291 |
| Sirt7 | NM_153056.2 | 0.0294 |
| Gnaq | NM_008139.5 | 0.0296 |
| Ugt8a | NM_011674.4 | 0.0297 |
| Amph | NM_175007.1 | 0.0303 |
| Gusb | NM_010368.1 | 0.0304 |
| Ikbkb | NM_010546.2 | 0.0307 |
| Htr5a | NM_008314.2 | 0.0310 |
| Aars | NM_146217.4 | 0.0311 |
| Taf6l | NM_001177798.1 | 0.0312 |
| Cers4 | NM_026058.4 | 0.0316 |
| Lars | NM_134137.2 | 0.0320 |
| Stx1a | NM_016801.3 | 0.0321 |
| Cacnb4 | NM_146123.2 | 0.0323 |
| Gtf2h1 | NM_008186.4 | 0.0337 |
| Ap2b1 | NM_001035854.2 | 0.0338 |
| Itgam | NM_008401.2 | 0.0342 |
| Tbr1 | NM_009322.3 | 0.0344 |
| Eng | NM_001146350.1 | 0.0358 |
| Insr | NM_010568.2 | 0.0372 |
| Gpr37 | NM_010338.2 | 0.0377 |
| Prkcq | NM_008859.2 | 0.0381 |
| Bace1 | NM_011792.4 | 0.0381 |
| Bcl2 | NM_009741.3 | 0.0386 |
| Il6 | NM_031168.1 | 0.0387 |
| Lrrc25 | NM_153074.3 | 0.0390 |
| Sox10 | XM 128139.6 | 0.0390 |
| Prpf31 | NM_001159714.1 | 0.0393 |
| Gnail | NM_010305.1 | 0.0396 |
| Ep300 | NM_177821.6 | 0.0401 |
| Akt3 | NM_011785.3 | 0.0402 |
| Pkn1 | NM_001199593.1 | 0.0403 |
| Gdnf | NM_010275.2 | 0.0425 |
| Polr2h | NM_145632.2 | 0.0427 |
| Shank2 | NM_001081370.2 | 0.0429 |
| Nmnat2 | NM_175460.3 | 0.0431 |
| Dlgap1 | NM_027712.3 | 0.0438 |
| Gad2 | NM_008078.2 | 0.0438 |
| Ccr5 | NM_009917.5 | 0.0441 |
| Flt4 | NM_008029.3 | 0.0441 |
| Fyn | NM_008054.2 | 0.0449 |
| Axin2 | NM_015732.4 | 0.0452 |
| Tlr4 | NM_021297.2 | 0.0456 |
| Pdpk1 | NM_001080773.2 | 0.0457 |
| Gad1 | NM_008077.4 | 0.0459 |
| Mmp19 | NM_021412.2 | 0.0460 |
| Gata2 | NM_008090.4 | 0.0460 |
| Snap91 | NM_001277986.1 | 0.0469 |
| Cacna1c | NM_001159535.1 | 0.0472 |
| Ptdss2 | NM_013782.4 | 0.0474 |
| Pik3ca | NM_008839.1 | 0.0474 |
| C1qa | NM_007572.2 | 0.0479 |
| Lclat1 | NM_001177967.1 | 0.0483 |
| Atp6v1g2 | NM_023179.3 | 0.0483 |
| Srsf4 | NM_020587.2 | 0.0484 |
| Cntnap2 | NM_001004357.2 | 0.0486 |
| Gnai2 | NM_008138.4 | 0.0487 |
| Sirt2 | NM_022432.4 | 0.0487 |
| Hpgds | NM_019455.4 | 0.0492 |
| Kcna1 | NM_010595.3 | 0.0499 |

TABLE 8
From TTPB_0400_final document,
Differentially Expressed Genes-DIV06

| Gene Name | Accession # | Pval |
|---|---|---|
| Smn1 | NM_011420.2 | 0.0000 |
| Slc2a1 | NM_011400.3 | 0.0000 |
| Nostrin | NM_181547.3 | 0.0000 |
| Nf1 | NM_010897.2 | 0.0000 |
| Cd44 | NM_009851.2 | 0.0000 |
| Hmoxl | NM_010442.2 | 0.0000 |
| Gal3st1 | NM_001177691.1 | 0.0000 |
| Atp8a2 | NM_015803.2 | 0.0000 |
| Serpinb6a | NM_001164117.1 | 0.0001 |
| Cntn1 | NM_001159647.1 | 0.0001 |
| Plxnb3 | NM_019587.2 | 0.0001 |
| Cntnap2 | NM_001004357.2 | 0.0001 |
| Ugt8a | NM_011674.4 | 0.0001 |
| Clu | NM_013492.2 | 0.0001 |
| Slc17a6 | NM_080853.3 | 0.0002 |
| Gstp1 | NM_013541.1 | 0.0002 |
| Lsm2 | NM_001110101.2 | 0.0002 |
| P2ry12 | NM_027571.3 | 0.0002 |
| Pla2g4a | NM_008869.2 | 0.0002 |
| Tmem119 | NM_146162.2 | 0.0002 |
| Negr1 | NM_001039094.2 | 0.0002 |
| Myrf | NM_001033481.1 | 0.0002 |
| Camk2g | NM_001039138.1 | 0.0002 |
| Grin2c | NM_010350.2 | 0.0002 |
| Cntnap1 | NM_016782.2 | 0.0002 |
| Pnkd | NM_019999.2 | 0.0002 |
| Igf1r | NM_010513.2 | 0.0002 |
| Cx3cr1 | NM_009987.3 | 0.0002 |
| Pfn1 | NM_011072.4 | 0.0003 |
| Slc12a5 | NM_020333.2 | 0.0003 |
| AI464131 | NM_001085515.2 | 0.0003 |
| Syt1 | NM_009306.2 | 0.0003 |
| Lsr | NM_001164184.1 | 0.0003 |
| Mtor | NM_020009.2 | 0.0003 |
| Il10ra | NM_008348.2 | 0.0003 |
| Taz | NM_181516.5 | 0.0004 |
| Insr | NM_010568.2 | 0.0004 |
| Wfs1 | NM_011716.2 | 0.0004 |
| Gabra1 | NM_010250.4 | 0.0004 |
| Bcas1 | NM_029815.2 | 0.0004 |
| Adrb2 | NM_007420.2 | 0.0004 |
| Cd9 | NM_007657.3 | 0.0004 |
| Ap4s1 | NM_021710.3 | 0.0004 |
| Apoe | NM_001305844.1 | 0.0004 |
| Vegfa | NM_001025250.3 | 0.0004 |
| Epha5 | NM_007937.3 | 0.0004 |
| Epha7 | NM_001122889.1 | 0.0004 |
| Cxcr4 | NM_009911.3 | 0.0004 |
| Cnr1 | NM_007726.3 | 0.0004 |
| Casp7 | NM_007611.2 | 0.0004 |
| Thyl | NM_009382.3 | 0.0005 |
| Htt | NM_010414.1 | 0.0005 |
| Grm8 | NM_008174.2 | 0.0005 |
| Synj1 | NM_001045515.1 | 0.0005 |
| Rasgrp1 | NM_011246.2 | 0.0005 |
| Casp3 | NM_009810.2 | 0.0005 |
| Itga5 | NM_001314041.1 | 0.0005 |
| Itpr1 | NM_010585.2 | 0.0005 |
| Gabra4 | NM_010251.2 | 0.0005 |
| Ptprn2 | NM_011215.2 | 0.0006 |
| Osmr | NM_011019.3 | 0.0006 |
| Csf1r | NM_001037859.1 | 0.0006 |
| Lama2 | NM_008481.2 | 0.0006 |
| Grm5 | NM_001143834.1 | 0.0007 |
| Cldn5 | NM_013805.4 | 0.0007 |
| Cdk5r1 | NM_009871.2 | 0.0007 |
| Plcb1 | NM_019677.1 | 0.0007 |
| Scn1a | NM_018733.2 | 0.0007 |
| Lmna | NM_001002011.2 | 0.0007 |
| Olig2 | NM_016967.2 | 0.0008 |
| Gabrb2 | NM_008070.3 | 0.0008 |
| Ccnd1 | NM_007631.1 | 0.0008 |
| Grin2b | NM_008171.3 | 0.0008 |
| Cdkn1a | NM_007669.4 | 0.0009 |
| Hdac2 | NM_008229.2 | 0.0009 |
| Cd68 | NM_009853.1 | 0.0010 |
| Cp | NM_001042611.1 | 0.0010 |
| Ppargc1a | NM_008904.2 | 0.0010 |
| Fa2h | NM_178086.3 | 0.0010 |
| Gabrb3 | NM_008071.3 | 0.0011 |
| Mmp12 | NM_008605.3 | 0.0011 |
| Cadm3 | NM_053199.3 | 0.0011 |
| Atp6v0e | NM_025272.2 | 0.0011 |
| Rab3c | NM_023852.5 | 0.0011 |
| Apaf1 | NM_009684.2 | 0.0012 |
| Sri | NM_001080974.2 | 0.0012 |
| Cx3cl1 | NM_009142.3 | 0.0012 |
| Egfr | NM_207655.2 | 0.0013 |
| Gnb5 | NM_138719.5 | 0.0013 |
| Kcnj10 | NM_001039484.1 | 0.0013 |
| Tspo | NM_009775.4 | 0.0013 |
| Fyn | NM_008054.2 | 0.0013 |
| Mag | NM_010758.2 | 0.0013 |
| Atm | NM_009730.2 | 0.0013 |
| Pmp | NM_011170.2 | 0.0013 |
| Dlg3 | NM_001177778.1 | 0.0013 |
| App | NM_007471.2 | 0.0014 |
| Calb1 | NM_009788.4 | 0.0014 |
| Grm1 | NM_001114333.2 | 0.0015 |
| Unc13a | NM_001029873.2 | 0.0015 |
| Tnfrsf12a | NM_001161746.1 | 0.0015 |
| Cds1 | NM_173370.3 | 0.0015 |
| Ep300 | NM_177821.6 | 0.0015 |
| Rapgef2 | NM_001099624.2 | 0.0016 |
| Fit1 | NM_010228.3 | 0.0016 |
| Slc9a6 | NM_172780.3 | 0.0016 |
| Ngf | NM_001112698.1 | 0.0016 |
| Slc4a10 | NM_001242380.1 | 0.0016 |
| Mmp2 | NM_008610.2 | 0.0018 |
| Sqstm1 | NM_011018.2 | 0.0018 |
| Mto1 | NM_026658.2 | 0.0018 |
| Cacna1b | NM_001042528.1 | 0.0018 |
| Bax | NM_007527.3 | 0.0018 |
| Gprasp1 | NM_001004359.2 | 0.0019 |
| Gdnf | NM_010275.2 | 0.0019 |
| Gria1 | NM_001252403.1 | 0.0019 |
| Atxn7 | NM_139227.4 | 0.0020 |
| Aars | NM_146217.4 | 0.0020 |
| Jam3 | NM_023277.4 | 0.0020 |
| Cul1 | NM_012042.3 | 0.0020 |
| Ptdss2 | NM_013782.4 | 0.0020 |
| PlCl2 | NM_013880.3 | 0.0020 |
| Pcsk2 | NM_008792.3 | 0.0021 |
| Islr2 | NM_001161538.1 | 0.0021 |
| Dagla | NM_198114.2 | 0.0021 |
| Snca | NM_009221.2 | 0.0021 |
| Ryr2 | NM_023868.1 | 0.0021 |
| Hdac1 | NM_008228.2 | 0.0022 |
| Cast | NM_009817.1 | 0.0022 |
| Prkce | NM_011104.2 | 0.0022 |
| Cycs | NM_007808.4 | 0.0022 |
| Gucy1b3 | NM_017469.4 | 0.0022 |
| Lrrk2 | NM_025730.3 | 0.0023 |
| Map2k1 | NM_008927.3 | 0.0023 |
| Sp100 | NM_013673.3 | 0.0023 |
| Kcnb1 | NM_008420.3 | 0.0024 |
| B4galt6 | NM_019737.2 | 0.0024 |
| Dgkb | NM_178681.4 | 0.0024 |
| Park2 | NM_016694.3 | 0.0025 |
| Rit2 | NM_009065.2 | 0.0026 |
| Fcrls | NM_030707.3 | 0.0026 |
| Camk2b | NM_001174053.1 | 0.0028 |
| Atp2b3 | NM_177236.3 | 0.0028 |
| Sorl1 | NM_011436.3 | 0.0028 |
| Frmpd4 | NM_001033330.2 | 0.0029 |
| Tada2b | NM_001170454.1 | 0.0029 |
| Gnptg | NM_172529.3 | 0.0029 |
| L1cam | NM_008478.3 | 0.0029 |
| Gria4 | NM_001113180.1 | 0.0029 |
| Il1r1 | NM_001123382.1 | 0.0029 |
| Pik3ca | NM_008839.1 | 0.0031 |
| Syt7 | NM_018801.3 | 0.0031 |
| Prkcg | NM_011102.3 | 0.0031 |
| Msn | NM_010833.2 | 0.0031 |
| Cck | NM_031161.2 | 0.0032 |
| Csf1 | NM_001113530.1 | 0.0032 |
| Cnksr2 | NM_177751.2 | 0.0032 |
| Fmr1 | NM_008031.2 | 0.0033 |
| Nptn | NM_009145.2 | 0.0033 |
| Hspb1 | NM_013560.2 | 0.0034 |
| Camk4 | NM_009793.3 | 0.0034 |
| Gls | NM_001113383.1 | 0.0034 |
| Gnao1 | NM_010308.3 | 0.0034 |
| Car2 | NM_009801.4 | 0.0036 |
| Prkcq | NM_008859.2 | 0.0036 |
| Nsf | NM_008740.2 | 0.0036 |
| Slc1a1 | NM_009199.2 | 0.0036 |
| Cxcl12 | NM_021704.3 | 0.0036 |
| Lamb2 | NM_008483.3 | 0.0036 |
| Scn2a1 | NM_001099298.3 | 0.0037 |
| Sncb | NM_033610.2 | 0.0037 |
| Kif3a | NM_008443.3 | 0.0037 |
| Cntf | NM_170786.2 | 0.0037 |
| Sorcs3 | NM_025696.3 | 0.0038 |
| Gabrg2 | NM_177408.5 | 0.0038 |
| Trp53 | NM_011640.1 | 0.0038 |
| Syt13 | NM_030725.4 | 0.0038 |
| Prkacb | NM_011100.3 | 0.0039 |
| Npy | NM_023456.2 | 0.0040 |
| Sox9 | NM_011448.4 | 0.0040 |
| Src | NM_001025395.2 | 0.0040 |
| Grin1 | NM_008169.2 | 0.0041 |
| Cacna1a | NM_007578.3 | 0.0041 |
| Eng | NM_001146350.1 | 0.0042 |
| Cers2 | NM_029789.1 | 0.0043 |
| Egr1 | NM_007913.5 | 0.0043 |
| Gad2 | NM_008078.2 | 0.0043 |
| Polr2k | NM_001039368.1 | 0.0043 |
| Jun | NM_010591.2 | 0.0043 |
| Rab3a | NM_009001.6 | 0.0044 |
| Gusb | NM_010368.1 | 0.0044 |
| Cdc40 | NM_027879.2 | 0.0044 |
| Esam | NM_027102.3 | 0.0045 |
| Vcp | NM_009503.3 | 0.0046 |
| Nrxn1 | NM_001346960.1 | 0.0047 |
| Hif1a | NM_010431.2 | 0.0047 |
| Dnml1 | NM_001025947.1 | 0.0049 |
| Fgf14 | NM_207667.3 | 0.0050 |
| Nwd1 | NM_176940.5 | 0.0050 |
| Stat3 | NM_213659.2 | 0.0051 |
| Pak1 | NM_011035.2 | 0.0051 |
| Cul3 | NM_016716.4 | 0.0052 |
| Cadps | NM_001042617.1 | 0.0053 |
| Olfm3 | NM_153157.3 | 0.0053 |
| Idh1 | NM_010497.2 | 0.0054 |
| Parp1 | NM_007415.2 | 0.0055 |
| Cxcl16 | NM_023158.6 | 0.0055 |
| Tie1 | NM_011587.2 | 0.0056 |
| Cacnb2 | NM_023116.3 | 0.0057 |
| Gpr37 | NM_010338.2 | 0.0058 |
| Xiap | NM_009688.2 | 0.0058 |
| Aqp4 | NM_009700.2 | 0.0058 |
| Tnfrsf10b | NM_020275.3 | 0.0058 |
| Slc1a2 | NM_001077514.3 | 0.0060 |
| Cntn4 | NM_001109751.1 | 0.0061 |
| Lif | NM_008501.2 | 0.0062 |
| Nell2 | NM_016743.2 | 0.0064 |
| Hgf | NM_010427.5 | 0.0065 |
| Gria2 | NM_001039195.1 | 0.0066 |
| Stx2 | NM_007941.2 | 0.0066 |
| Bid | NM_007544.3 | 0.0066 |
| Gfap | NM_001131020.1 | 0.0066 |
| Abat | NM_001170978.1 | 0.0066 |
| Sirt2 | NM_022432.4 | 0.0067 |
| Hcn1 | NM_010408.3 | 0.0067 |
| Slc8a1 | NM_011406.2 | 0.0068 |
| Gsr | NM_010344.4 | 0.0069 |
| Ppp3cc | NM_008915.2 | 0.0070 |
| Arhgef10 | NM_001037736.1 | 0.0070 |
| Amigo1 | NM_001004293.2 | 0.0071 |
| Eif2s1 | NM_026114.3 | 0.0071 |
| Gria3 | NM_016886.3 | 0.0071 |
| Sucla2 | NM_011506.1 | 0.0073 |
| Mapk1 | NM_011949.3 | 0.0075 |
| Psen2 | NM_001128605.1 | 0.0076 |
| Ggt1 | NM_008116.2 | 0.0076 |
| Epha6 | NM_007938.2 | 0.0077 |
| Mmp9 | NM_013599.2 | 0.0077 |
| Ran | NM_009391.3 | 0.0078 |
| Sgpl1 | NM_009163.3 | 0.0079 |
| Cd33 | NM_001111058.1 | 0.0080 |
| Nos1 | NM_008712.2 | 0.0082 |
| Plcb4 | NM_013829.2 | 0.0084 |
| Ccr5 | NM_009917.5 | 0.0085 |
| Cxcl10 | NM_021274.1 | 0.0085 |
| Magee1 | NM_053201.3 | 0.0086 |
| Mfn2 | XM_006535920.1 | 0.0087 |
| Ugcg | NM_011673.3 | 0.0088 |
| Slc11a1 | NM_013612.2 | 0.0088 |
| Ptgs2 | NM_011198.3 | 0.0089 |
| Mmp14 | NM_008608.3 | 0.0089 |
| Gaa | NM_008064.3 | 0.0089 |
| Igf1 | NM_010512.4 | 0.0091 |
| Chmb2 | NM_009602.4 | 0.0092 |
| Mbp | NM_010777.3 | 0.0093 |
| Ringl | NM_009066.3 | 0.0093 |
| Cd34 | NM_001111059.1 | 0.0093 |
| Lsm7 | NM_025349.2 | 0.0095 |
| Gata2 | NM_008090.4 | 0.0096 |
| Stxla | NM_016801.3 | 0.0097 |
| Fgf12 | NM_001276419.1 | 0.0097 |
| Egf17 | NM_001164564.1 | 0.0097 |
| Axin2 | NM_015732.4 | 0.0098 |
| Ikbkb | NM_010546.2 | 0.0099 |
| Glrb | NM_010298.5 | 0.0099 |
| Acvrll | NM_009612.2 | 0.0100 |
| Pena | NM_011045.2 | 0.0101 |
| Aktl | NM_001165894.1 | 0.0101 |
| Inpp4a | NM_172971.2 | 0.0101 |
| Crebbp | NM_001025432.1 | 0.0102 |
| Prkcb | NM_008855.2 | 0.0102 |
| Creb1 | NM_001037726.1 | 0.0102 |
| Slu7 | NM_148673.3 | 0.0102 |
| Ppp3ca | NM_008913.4 | 0.0103 |
| Man2b1 | NM_010764.2 | 0.0103 |
| Cacna1d | NM_028981.2 | 0.0103 |
| Mapk10 | NM_001081567.1 | 0.0103 |
| Akt3 | NM_011785.3 | 0.0103 |
| Sod2 | NM_013671.3 | 0.0104 |
| Tnr | NM_022312.3 | 0.0104 |
| Tenm2 | NM_011856.3 | 0.0107 |
| Epha4 | NM_007936.3 | 0.0108 |
| Cln8 | NM_012000.3 | 0.0110 |
| Emcn | NM_001163522.1 | 0.0112 |
| P2rx4 | NM_011026.2 | 0.0112 |
| Phf19 | NM_028716.4 | 0.0113 |
| Gpdl1 | NM_175380.5 | 0.0113 |
| Nrg1 | NM_178591.2 | 0.0113 |
| Prkaa2 | NM_178143.1 | 0.0115 |
| Grin2a | NM_008170.2 | 0.0115 |
| Gsn | NM_146120.3 | 0.0116 |
| Tnfrsf1a | NM_011609.2 | 0.0117 |
| Optn | NM_181848.4 | 0.0117 |
| Nef1 | NM_010910.1 | 0.0118 |
| Ipcef1 | NM_001033391.2 | 0.0119 |
| Lrp1 | NM_008512.2 | 0.0119 |
| Myc | NM_010849.4 | 0.0121 |
| Angpt2 | NM_007426.3 | 0.0121 |
| Tardbp | NM_001003899.2 | 0.0121 |
| C3 | XM_011246258.1 | 0.0122 |
| Stambpl1 | NM_029682.4 | 0.0122 |
| Stxlb | NM_024414.2 | 0.0123 |
| Gba | NM_001077411.1 | 0.0123 |
| Ncaml | NM_001113204.1 | 0.0124 |
| Amph | NM_175007.1 | 0.0124 |
| Icaml | NM_010493.2 | 0.0128 |
| Mapk8 | NM_016700.3 | 0.0128 |
| Tnfrsf11b | NM_008764.3 | 0.0128 |
| Chrna7 | NM_007390.3 | 0.0129 |
| Ptdss1 | NM_008959.3 | 0.0129 |
| Adcy8 | NM_009623.2 | 0.0129 |
| Trim37 | NM_197987.2 | 0.0129 |
| Tbp | NM_013684.3 | 0.0130 |
| Cybb | NM_007807.2 | 0.0131 |
| Emp2 | NM_007929.2 | 0.0139 |
| Gsk3b | NM_019827.3 | 0.0140 |
| Ang | NM_007447.2 | 0.0140 |
| Bcl2l1 | NM_009743.4 | 0.0141 |
| Grm2 | NM_001160353.1 | 0.0143 |
| Drdl | NM_010076.3 | 0.0144 |
| Shank2 | NM_001081370.2 | 0.0144 |
| Cd14 | NM_009841.3 | 0.0145 |
| Sec23a | NM_009147.2 | 0.0149 |
| Atpl3a2 | NM_029097.2 | 0.0149 |
| Ube2n | NM_080560.3 | 0.0151 |
| Dgke | NM_019505.3 | 0.0152 |
| Erbb3 | NM_010153.1 | 0.0152 |
| Atcay | NM_178662.3 | 0.0154 |
| Prpf31 | NM_001159714.1 | 0.0156 |
| Sirtl | NM_019812.2 | 0.0157 |
| Fgf2 | NM_008006.2 | 0.0157 |
| Adcy9 | NM_009624.1 | 0.0157 |
| Col4a2 | NM_009932.3 | 0.0158 |
| Dlat | NM_145614.4 | 0.0160 |
| Gtf2h3 | NM_181410.3 | 0.0161 |
| Snap9l | NM_001277986.1 | 0.0162 |
| Hdac7 | NM_019572.2 | 0.0163 |
| Hpgds | NM_019455.4 | 0.0163 |
| Tbpll | NM_011603.5 | 0.0163 |
| Map2 | NM_008632.2 | 0.0164 |
| Myh10 | NM_175260.2 | 0.0165 |
| Gng2 | NM_010315.4 | 0.0165 |
| Xbpl | NM_013842.2 | 0.0165 |
| Gadi | NM_008077.4 | 0.0171 |
| I14ra | NM_001008700.3 | 0.0175 |
| Efnal | NM_010107.4 | 0.0176 |
| Csf2rb | NM_007780.4 | 0.0177 |
| Entpd2 | NM_009849.2 | 0.0180 |
| Tnc | NM_011607.1 | 0.0182 |
| Trim28 | NM_011588.3 | 0.0185 |
| Pdpkl | NM_001080773.2 | 0.0189 |
| Oxri | NM_001130163.1 | 0.0190 |
| Mgmt | NM_008598.2 | 0.0191 |
| Psen1 | NM_008943.2 | 0.0191 |
| Nes | NM_016701.3 | 0.0192 |
| Caspl | NM_009807.2 | 0.0193 |
| Lars | NM_134137.2 | 0.0196 |
| Gjb1 | NM_008124.2 | 0.0198 |
| Chl1 | NM_007697.2 | 0.0198 |
| Ccdc127 | NM_024201.3 | 0.0198 |
| Ppp3cb | NM_008914.1 | 0.0200 |
| Atp6v0c | NM_009729.3 | 0.0201 |
| Calm1 | NM_009790.4 | 0.0207 |
| Efna5 | NM_207654.2 | 0.0208 |
| Efr3a | NM_133766.3 | 0.0209 |
| Sptbn2 | NM_021287.1 | 0.0210 |
| Vip | NM_011702.2 | 0.0216 |
| Txnl1 | NM_016792.2 | 0.0217 |
| Atp6v1g2 | NM_023179.3 | 0.0218 |
| Sox10 | XM_128139.6 | 0.0220 |
| Dlgap1 | NM_027712.3 | 0.0220 |
| Htr1a | NM_008308.4 | 0.0222 |
| Cers6 | NM_172856.3 | 0.0223 |
| Katna1 | NM_011835.2 | 0.0223 |
| Tcirg1 | NM_001136091.1 | 0.0230 |
| Prkaca | NM_008854.3 | 0.0235 |
| Nova1 | NM_021361.1 | 0.0235 |
| Itgam | NM_008401.2 | 0.0236 |
| Taf6l | NM_001177798.1 | 0.0237 |
| Aldh1l1 | NM_027406.1 | 0.0238 |
| Nlrp3 | NM_145827.3 | 0.0238 |
| Lox | NM_010728.2 | 0.0238 |
| Arhgap44 | NM_001099288.1 | 0.0239 |
| Ap2b1 | NM_001035854.2 | 0.0245 |
| Tnfrsf1b | NM_011610.3 | 0.0248 |
| Cyp4x1 | NM_001003947.1 | 0.0249 |
| I16ra | NM_010559.2 | 0.0251 |
| Nfe2l2 | NR_132727.1 | 0.0251 |
| Ina | NM_146100.4 | 0.0252 |
| Mmp19 | NM_021412.2 | 0.0253 |
| Homer1 | NM_147176.2 | 0.0256 |
| Tnf | NM_013693.2 | 0.0258 |
| Slc32a1 | NM_009508.2 | 0.0258 |
| Cacna1c | NM_001159535.1 | 0.0261 |
| Cacnb4 | NM_146123.2 | 0.0261 |
| Cd40 | NM_011611.2 | 0.0262 |
| Fos | NM_010234.2 | 0.0265 |
| Mecp2 | NM_010788.2 | 0.0266 |
| Ap2a2 | NM_007459.3 | 0.0269 |
| Pmp22 | NM_008885.2 | 0.0270 |
| Taf10 | NM_020024.3 | 0.0271 |
| Pde1b | NM_008800.1 | 0.0276 |
| Erg | NM_133659.2 | 0.0277 |
| Ptprr | NM_011217.2 | 0.0283 |
| Ap3m2 | NM_029505.3 | 0.0296 |
| Pla2g16 | NM_139269.2 | 0.0305 |
| Cplx1 | NM_007756.3 | 0.0306 |
| Nmnat2 | NM_175460.3 | 0.0308 |
| Gnaq | NM_008139.5 | 0.0311 |
| Grik2 | NM_010349.2 | 0.0314 |
| Tbr1 | NM_009322.3 | 0.0315 |
| Sf3a2 | NM_013651.4 | 0.0317 |
| Fam104a | NM_138598.5 | 0.0319 |
| Mta2 | NM_011842.3 | 0.0320 |
| Acin1 | NM_001085472.2 | 0.0321 |
| Rhoa | NM_016802.4 | 0.0323 |
| Atp6v0e2 | NM_133764.3 | 0.0339 |
| Gss | NM_008180.1 | 0.0342 |
| Gnai1 | NM_010305.1 | 0.0343 |
| Xab2 | NM_026156.2 | 0.0344 |
| Erlec1 | NM_025745.3 | 0.0355 |
| Lrrc25 | NM_153074.3 | 0.0357 |
| Naglu | NM_013792.2 | 0.0358 |
| Pla2g4b | XM_925095.2 | 0.0362 |
| Nqo1 | NM_008706.5 | 0.0364 |
| Cnot10 | NM_153585.5 | 0.0365 |
| Lrrc4 | NM_138682.2 | 0.0367 |
| Cers4 | NM_026058.4 | 0.0368 |
| Hexb | NM_010422.2 | 0.0369 |
| Nr4a2 | NM_001139509.1 | 0.0370 |
| Plekho2 | NM_153119.2 | 0.0377 |
| Mapk9 | NM_207692.1 | 0.0379 |
| Arsa | NM_009713.4 | 0.0385 |
| Comt | NM_007744.3 | 0.0395 |
| Gnai3 | NM_010306.2 | 0.0397 |
| Hras | NM_001130443.1 | 0.0399 |
| Cdk2 | NM_016756.4 | 0.0402 |
| Ddit3 | NM_007837.3 | 0.0402 |
| Tgfb1 | NM_011577.1 | 0.0409 |
| Opa1 | NM_001199177.1 | 0.0410 |
| Pgk1 | NM_008828.2 | 0.0413 |
| Atxn2 | NM_009125.2 | 0.0421 |
| Trf | NM_133977.2 | 0.0429 |
| Gfpt1 | NM_013528.3 | 0.0434 |
| Atp6v1d | NM_023721.2 | 0.0435 |
| Trpm2 | NM_138301.2 | 0.0437 |
| Ltbr | NM_010736.3 | 0.0439 |
| Ube2k | NM_016786.3 | 0.0442 |
| Ppp2r5e | NM_012024.2 | 0.0444 |
| Hmgb1 | NM_010439.3 | 0.0459 |
| Gnai2 | NM_008138.4 | 0.0468 |
| Grn | NM_008175.3 | 0.0476 |
| Srsf4 | NM_020587.2 | 0.0476 |
| Gpr4 | NM_175668.4 | 0.0478 |
| Chd4 | NM_145979.2 | 0.0487 |
| Ryr1 | NM_009109.1 | 0.0488 |
| Ctns | NM_031251.4 | 0.0493 |
| Calb2 | NM_007586.1 | 0.0499 |
| Ide | NM_031156.2 | 0.0501 |

TABLE 9
From TTPB_0400_final document,
Differentially Expressed Genes-DIV08

| Gene Name | Accession # | Pval |
|---|---|---|
| Ep300 | NM_177821.6 | 0.0000 |
| Hmoxl | NM_010442.2 | 0.0000 |
| Cd44 | NM_009851.2 | 0.0000 |
| Adrb2 | NM_007420.2 | 0.0002 |
| Gabrb2 | NM_008070.3 | 0.0002 |
| Atp2b3 | NM_177236.3 | 0.0002 |
| Cacna1b | NM_001042528.1 | 0.0002 |
| C3 | XM_011246258.1 | 0.0002 |
| Cdk5r1 | NM_009871.2 | 0.0002 |
| Pnkd | NM_019999.2 | 0.0002 |
| Camk2g | NM_001039138.1 | 0.0003 |
| AI464131 | NM_001085515.2 | 0.0003 |
| Serpinb6a | NM_001164117.1 | 0.0003 |
| P2ry12 | NM_027571.3 | 0.0003 |
| Plcb1 | NM_019677.1 | 0.0003 |
| Vegfa | NM_001025250.3 | 0.0003 |
| Negr1 | NM_001039094.2 | 0.0004 |
| Mmp2 | NM_008610.2 | 0.0004 |
| Gal3st1 | NM_001177691.1 | 0.0004 |
| Cxcr4 | NM_009911.3 | 0.0004 |
| Gabra1 | NM_010250.4 | 0.0004 |
| Taz | NM_181516.5 | 0.0005 |
| Gria1 | NM_001252403.1 | 0.0005 |
| Csf1 | NM_001113530.1 | 0.0005 |
| Beast | NM_029815.2 | 0.0005 |
| Atm | NM_009730.2 | 0.0005 |
| Cdkn1a | NM_007669.4 | 0.0005 |
| Cend1 | NM_007631.1 | 0.0006 |
| Tmem119 | NM_146162.2 | 0.0006 |
| Grm5 | NM_001143834.1 | 0.0006 |
| Htt | NM_010414.1 | 0.0006 |
| Slc12a5 | NM_020333.2 | 0.0006 |
| Gprasp1 | NM_001004359.2 | 0.0006 |
| Sorl1 | NM_011436.3 | 0.0007 |
| Ptprn2 | NM_011215.2 | 0.0007 |
| Csf1r | NM_001037859.1 | 0.0007 |
| Cd9 | NM_007657.3 | 0.0007 |
| Nrxn1 | NM_001346960.1 | 0.0007 |
| Slc9a6 | NM_172780.3 | 0.0009 |
| Cx3cl1 | NM_009142.3 | 0.0009 |
| Itga5 | NM_001314041.1 | 0.0009 |
| Atp8a2 | NM_015803.2 | 0.0010 |
| Thy1 | NM_009382.3 | 0.0012 |
| Grin2c | NM_010350.2 | 0.0012 |
| Gnb5 | NM_138719.5 | 0.0012 |
| Gba | NM_001077411.1 | 0.0013 |
| Plxnb3 | NM_019587.2 | 0.0013 |
| Slc4a10 | NM_001242380.1 | 0.0013 |
| Cp | NM_001042611.1 | 0.0013 |
| Mtor | NM_020009.2 | 0.0013 |
| Mmp12 | NM_008605.3 | 0.0014 |
| Grin2b | NM_008171.3 | 0.0014 |
| Mag | NM_010758.2 | 0.0014 |
| Abat | NM_001170978.1 | 0.0014 |
| Pcsk2 | NM_008792.3 | 0.0014 |
| Amigo1 | NM_001004293.2 | 0.0015 |
| Gabra4 | NM_010251.2 | 0.0015 |
| Cadm3 | NM_053199.3 | 0.0015 |
| Rasgrp1 | NM_011246.2 | 0.0015 |
| Ryr2 | NM_023868.1 | 0.0015 |
| Frmpd4 | NM_001033330.2 | 0.0015 |
| Jun | NM_010591.2 | 0.0015 |
| Prkcg | NM_011102.3 | 0.0016 |
| Gsr | NM_010344.4 | 0.0017 |
| Syt1 | NM_009306.2 | 0.0018 |
| Dgkb | NM_178681.4 | 0.0018 |
| Grin1 | NM_008169.2 | 0.0019 |
| Rab3c | NM_023852.5 | 0.0019 |
| Prkacb | NM_011100.3 | 0.0019 |
| Tspo | NM_009775.4 | 0.0019 |
| Gabrb3 | NM_008071.3 | 0.0020 |
| Igf1r | NM_010513.2 | 0.0020 |
| Glib | NM_010298.5 | 0.0021 |
| Sp100 | NM_013673.3 | 0.0023 |
| Nsf | NM_008740.2 | 0.0025 |
| Fer1s | NM_030707.3 | 0.0025 |
| Dag1a | NM_198114.2 | 0.0025 |
| Lmna | NM_001002011.2 | 0.0026 |
| Epha4 | NM_007936.3 | 0.0026 |
| Ptdss1 | NM_008959.3 | 0.0026 |
| Jam3 | NM_023277.4 | 0.0027 |
| Nptn | NM_009145.2 | 0.0027 |
| Prkce | NM_011104.2 | 0.0028 |
| Calb1 | NM_009788.4 | 0.0028 |
| Gnao1 | NM_010308.3 | 0.0029 |
| Cx3cr1 | NM_009987.3 | 0.0029 |
| Gabrg2 | NM_177408.5 | 0.0030 |
| Nes | NM_016701.3 | 0.0030 |
| Cnr1 | NM_007726.3 | 0.0031 |
| Flt1 | NM_010228.3 | 0.0031 |
| Ptgs2 | NM_011198.3 | 0.0031 |
| Gria3 | NM_016886.3 | 0.0032 |
| Scn2a1 | NM_001099298.3 | 0.0032 |
| Epha5 | NM_007937.3 | 0.0032 |
| Ncam1 | NM_001113204.1 | 0.0033 |
| Snea | NM_009221.2 | 0.0034 |
| Mapk10 | NM_001081567.1 | 0.0035 |
| Dlg3 | NM_001177778.1 | 0.0036 |
| Cnksr2 | NM_177751.2 | 0.0036 |
| Emp2 | NM_007929.2 | 0.0036 |
| Cd68 | NM_009853.1 | 0.0036 |
| Gnptg | NM_172529.3 | 0.0037 |
| Cast | NM_009817.1 | 0.0037 |
| Galc | NM_008079.3 | 0.0037 |
| Il6ra | NM_010559.2 | 0.0037 |
| Trp53 | NM_011640.1 | 0.0039 |
| Cxcl12 | NM_021704.3 | 0.0039 |
| Kif3a | NM_008443.3 | 0.0039 |
| Insr | NM_010568.2 | 0.0039 |
| Nwd1 | NM_176940.5 | 0.0041 |
| Rapgef2 | NM_001099624.2 | 0.0041 |
| Tbpl1 | NM_011603.5 | 0.0042 |
| Gria2 | NM_001039195.1 | 0.0042 |
| Gucy1b3 | NM_017469.4 | 0.0042 |
| Ggt1 | NM_008116.2 | 0.0043 |
| Ppp3ca | NM_008913.4 | 0.0044 |
| Fgf2 | NM_008006.2 | 0.0044 |
| Polr2k | NM_001039368.1 | 0.0044 |
| Epha7 | NM_001122889.1 | 0.0045 |
| Arhgef10 | NM_001037736.1 | 0.0045 |
| Cntnap1 | NM_016782.2 | 0.0045 |
| Apoe | NM_001305844.1 | 0.0046 |
| Lsm2 | NM_001110101.2 | 0.0046 |
| Gria4 | NM_001113180.1 | 0.0047 |
| Casp1 | NM_009807.2 | 0.0048 |
| Camk2b | NM_001174053.1 | 0.0049 |
| Grm2 | NM_001160353.1 | 0.0049 |
| Rab3a | NM_009001.6 | 0.0050 |
| Hcn1 | NM_010408.3 | 0.0051 |
| Dgke | NM_019505.3 | 0.0051 |
| Casp3 | NM_009810.2 | 0.0051 |
| App | NM_007471.2 | 0.0052 |
| Slc1a2 | NM_001077514.3 | 0.0052 |
| Tnfrsf12a | NM_001161746.1 | 0.0052 |
| Pfn1 | NM_011072.4 | 0.0053 |
| Pla2g4a | NM_008869.2 | 0.0053 |
| Magee1 | NM_053201.3 | 0.0054 |
| Cck | NM_031161.2 | 0.0054 |
| Slc8a1 | NM_011406.2 | 0.0055 |
| Gaa | NM_008064.3 | 0.0056 |
| Gtf2h3 | NM_181410.3 | 0.0056 |
| Gfap | NM_001131020.1 | 0.0056 |
| Cadps | NM_001042617.1 | 0.0056 |
| Synj1 | NM_001045515.1 | 0.0056 |
| Cybb | NM_007807.2 | 0.0057 |
| Atp6v0e | NM_025272.2 | 0.0058 |
| Myrf | NM_001033481.1 | 0.0059 |
| Entpd2 | NM_009849.2 | 0.0059 |
| Gls | NM_001113383.1 | 0.0060 |
| Grin2a | NM_008170.2 | 0.0061 |
| Tnr | NM_022312.3 | 0.0061 |
| Axin2 | NM_015732.4 | 0.0062 |
| Islr2 | NM_001161538.1 | 0.0062 |
| Bax | NM_007527.3 | 0.0063 |
| Egr1 | NM_007913.5 | 0.0064 |
| Unc13a | NM_001029873.2 | 0.0064 |
| Npy | NM_023456.2 | 0.0066 |
| Rit2 | NM_009065.2 | 0.0066 |
| Mbp | NM_010777.3 | 0.0066 |
| Myh10 | NM_175260.2 | 0.0068 |
| Ap2a2 | NM_007459.3 | 0.0070 |
| Syt13 | NM_030725.4 | 0.0071 |
| Lamb2 | NM_008483.3 | 0.0071 |
| Xbp1 | NM_013842.2 | 0.0072 |
| Grm1 | NM_001114333.2 | 0.0072 |
| Dlat | NM_145614.4 | 0.0074 |
| Atcay | NM_178662.3 | 0.0074 |
| Fgf12 | NM_001276419.1 | 0.0075 |
| Lox | NM_010728.2 | 0.0076 |
| Msn | NM_010833.2 | 0.0076 |
| Fa2h | NM_178086.3 | 0.0078 |
| Cers2 | NM_029789.1 | 0.0079 |
| Itgam | NM_008401.2 | 0.0081 |
| Ugt8a | NM_011674.4 | 0.0082 |
| Cacnala | NM_007578.3 | 0.0082 |
| P2rx4 | NM_011026.2 | 0.0082 |
| Cd33 | NM_001111058.1 | 0.0082 |
| Plcl2 | NM_013880.3 | 0.0083 |
| Mmp14 | NM_008608.3 | 0.0083 |
| Hspb1 | NM_013560.2 | 0.0083 |
| Clu | NM_013492.2 | 0.0086 |
| Nqo1 | NM_008706.5 | 0.0086 |
| Icam1 | NM_010493.2 | 0.0086 |
| Grm8 | NM_008174.2 | 0.0086 |
| Pak1 | NM_011035.2 | 0.0088 |
| Prnp | NM_011170.2 | 0.0088 |
| Atxn7 | NM_139227.4 | 0.0088 |
| Bcl211 | NM_009743.4 | 0.0090 |
| Cxcl10 | NM_021274.1 | 0.0091 |
| Scn1a | NM_018733.2 | 0.0091 |
| Cds1 | NM_173370.3 | 0.0091 |
| Adcy8 | NM_009623.2 | 0.0094 |
| Nos1 | NM_008712.2 | 0.0095 |
| B4galt6 | NM_019737.2 | 0.0096 |
| Map2k1 | NM_008927.3 | 0.0096 |
| Dnml1 | NM_001025947.1 | 0.0097 |
| Ran | NM_009391.3 | 0.0098 |
| Kcnb1 | NM_008420.3 | 0.0098 |
| Gstp1 | NM_013541.1 | 0.0099 |
| Illr1 | NM_001123382.1 | 0.0100 |
| Sirt2 | NM_022432.4 | 0.0100 |
| Camk4 | NM_009793.3 | 0.0101 |
| Ppargc1a | NM_008904.2 | 0.0101 |
| L1cam | NM_008478.3 | 0.0102 |
| Stx1a | NM_016801.3 | 0.0102 |
| Gsn | NM_146120.3 | 0.0102 |
| Pkn1 | NM_001199593.1 | 0.0103 |
| Gpdl1 | NM_175380.5 | 0.0103 |
| Erbb3 | NM_010153.1 | 0.0103 |
| Nell2 | NM_016743.2 | 0.0104 |
| Slc1a1 | NM_009199.2 | 0.0104 |
| Stat3 | NM_213659.2 | 0.0105 |
| Cxcl16 | NM_023158.6 | 0.0106 |
| Lsr | NM_001164184.1 | 0.0106 |
| Ppp3cb | NM_008914.1 | 0.0106 |
| Man2b1 | NM_010764.2 | 0.0109 |
| Myc | NM_010849.4 | 0.0112 |
| Mapk8 | NM_016700.3 | 0.0113 |
| Atp13a2 | NM_029097.2 | 0.0113 |
| Src | NM_001025395.2 | 0.0114 |
| Aldh111 | NM_027406.1 | 0.0116 |
| Ccr5 | NM_009917.5 | 0.0116 |
| Ipcef1 | NM_001033391.2 | 0.0117 |
| Ring1 | NM_009066.3 | 0.0120 |
| Nostrin | NM_181547.3 | 0.0120 |
| Tnfrsf1a | NM_011609.2 | 0.0121 |
| Eng | NM_001146350.1 | 0.0121 |
| Sncb | NM_033610.2 | 0.0121 |
| Tenm2 | NM_011856.3 | 0.0123 |
| Smn1 | NM_011420.2 | 0.0123 |
| Syt7 | NM_018801.3 | 0.0125 |
| Ube2n | NM_080560.3 | 0.0127 |
| Inpp4a | NM_172971.2 | 0.0128 |
| Txnl1 | NM_016792.2 | 0.0129 |
| Eif2s1 | NM_026114.3 | 0.0131 |
| Cacna1d | NM_028981.2 | 0.0131 |
| Lsm7 | NM_025349.2 | 0.0132 |
| Arsa | NM_009713.4 | 0.0133 |
| Gnaq | NM_008139.5 | 0.0137 |
| Wfs1 | NM_011716.2 | 0.0138 |
| Prkcb | NM_008855.2 | 0.0139 |
| Tada2b | NM_001170454.1 | 0.0139 |
| Prpf31 | NM_001159714.1 | 0.0139 |
| Plcb4 | NM_013829.2 | 0.0142 |
| Ap2b1 | NM_001035854.2 | 0.0144 |
| Itpr1 | NM_010585.2 | 0.0145 |
| Lrrk2 | NM_025730.3 | 0.0146 |
| Adcy9 | NM_009624.1 | 0.0147 |
| Ptdss2 | NM_013782.4 | 0.0148 |
| Tbp | NM_013684.3 | 0.0149 |
| Ina | NM_146100.4 | 0.0149 |
| Psen2 | NM_001128605.1 | 0.0151 |
| Tnc | NM_011607.1 | 0.0154 |
| Osmr | NM_011019.3 | 0.0155 |
| Adora1 | NM_001008533.3 | 0.0155 |
| Atp6v1g2 | NM_023179.3 | 0.0157 |
| Pdpk1 | NM_001080773.2 | 0.0160 |
| Ugcg | NM_011673.3 | 0.0161 |
| Cers4 | NM_026058.4 | 0.0162 |
| Angpt2 | NM_007426.3 | 0.0164 |
| Chrna7 | NM_007390.3 | 0.0165 |
| Mapk1 | NM_011949.3 | 0.0166 |
| Nmnat2 | NM_175460.3 | 0.0167 |
| Sqstm1 | NM_011018.2 | 0.0168 |
| Ikbkb | NM_010546.2 | 0.0168 |
| Atp6v0e2 | NM_133764.3 | 0.0168 |
| Cntn1 | NM_001159647.1 | 0.0168 |
| Mog | NM_010814.2 | 0.0170 |
| Fgf14 | NM_207667.3 | 0.0171 |
| Ppp3cc | NM_008915.2 | 0.0174 |
| Lrrc4 | NM_138682.2 | 0.0175 |
| Cdc40 | NM_027879.2 | 0.0178 |
| Ngf | NM_001112698.1 | 0.0178 |
| Olig2 | NM_016967.2 | 0.0179 |
| Tnfrsf10b | NM_020275.3 | 0.0180 |
| Casp7 | NM_007611.2 | 0.0182 |
| Fyn | NM_008054.2 | 0.0183 |
| Slc2a1 | NM_011400.3 | 0.0185 |
| Drd1 | NM_010076.3 | 0.0193 |
| Gng2 | NM_010315.4 | 0.0193 |
| Amph | NM_175007.1 | 0.0193 |
| Sorcs3 | NM_025696.3 | 0.0196 |
| Mfn2 | XM_006535920.1 | 0.0197 |
| Hdac1 | NM_008228.2 | 0.0198 |
| Snap91 | NM_001277986.1 | 0.0212 |
| Sptbn2 | NM_021287.1 | 0.0213 |
| Lama2 | NM_008481.2 | 0.0213 |
| Chl1 | NM_007697.2 | 0.0213 |
| Gusb | NM_010368.1 | 0.0218 |
| Vcp | NM_009503.3 | 0.0218 |
| Il10ra | NM_008348.2 | 0.0221 |
| Sgpl1 | NM_009163.3 | 0.0223 |
| Snrpa | NM_001046637.1 | 0.0227 |
| Gad2 | NM_008078.2 | 0.0228 |
| Park2 | NM_016694.3 | 0.0229 |
| Gjb1 | NM_008124.2 | 0.0230 |
| Nos3 | NM_008713.4 | 0.0232 |
| Atp6v1d | NM_023721.2 | 0.0233 |
| Lif | NM_008501.2 | 0.0241 |
| Shank2 | NM_001081370.2 | 0.0245 |
| Sirt7 | NM_153056.2 | 0.0245 |
| Chmb2 | NM_009602.4 | 0.0247 |
| Arrb2 | NM_145429.4 | 0.0249 |
| Cyp4x1 | NM_001003947.1 | 0.0249 |
| Ang | NM_007447.2 | 0.0249 |
| Cntn4 | NM_001109751.1 | 0.0250 |
| Atp6v0c | NM_009729.3 | 0.0252 |
| Fas | NM_007987.2 | 0.0254 |
| Lpar1 | NM_010336.2 | 0.0256 |
| Ap4s1 | NM_021710.3 | 0.0256 |
| Taf9 | NM_027139.5 | 0.0259 |
| Egfl7 | NM_001164564.1 | 0.0259 |
| Ccl12 | NM_011331.2 | 0.0260 |
| Kcnj10 | NM_001039484.1 | 0.0263 |
| Oxr1 | NM_001130163.1 | 0.0265 |
| Hgf | NM_010427.5 | 0.0266 |
| Mto1 | NM_026658.2 | 0.0270 |
| Aqp4 | NM_009700.2 | 0.0271 |
| Vip | NM_011702.2 | 0.0274 |
| Sox9 | NM_011448.4 | 0.0275 |
| Dlgap1 | NM_027712.3 | 0.0277 |
| Pde1b | NM_008800.1 | 0.0279 |
| Cacnb2 | NM_023116.3 | 0.0280 |
| Efna5 | NM_207654.2 | 0.0284 |
| Prkaa2 | NM_178143.1 | 0.0284 |
| Igf1 | NM_010512.4 | 0.0284 |
| C1qb | NM_009777.2 | 0.0285 |
| Naglu | NM_013792.2 | 0.0285 |
| Ap3m2 | NM_029505.3 | 0.0289 |
| Egfr | NM_207655.2 | 0.0289 |
| Homer1 | NM_147176.2 | 0.0291 |
| Map2 | NM_008632.2 | 0.0297 |
| Mapkapk2 | NM_008551.1 | 0.0298 |
| Sf3a2 | NM_013651.4 | 0.0298 |
| Cntnap2 | NM_001004357.2 | 0.0299 |
| Crebbp | NM_001025432.1 | 0.0306 |
| Fos | NM_010234.2 | 0.0307 |
| Slc32a1 | NM_009508.2 | 0.0311 |
| Sod2 | NM_013671.3 | 0.0313 |
| Stx1b | NM_024414.2 | 0.0315 |
| Fxn | NM_008044.2 | 0.0321 |
| Cacnb4 | NM_146123.2 | 0.0322 |
| Sucla2 | NM_011506.1 | 0.0323 |
| Apafl | NM_009684.2 | 0.0331 |
| Prkcq | NM_008859.2 | 0.0332 |
| Acvrll | NM_009612.2 | 0.0332 |
| Mmp19 | NM_021412.2 | 0.0333 |
| Efr3a | NM_133766.3 | 0.0334 |
| Atxn2 | NM_009125.2 | 0.0335 |
| Sirt1 | NM_019812.2 | 0.0339 |
| Slclla1 | NM_013612.2 | 0.0341 |
| Ppml1 | NM_178726.3 | 0.0343 |
| Hexb | NM_010422.2 | 0.0346 |
| Cln8 | NM_012000.3 | 0.0350 |
| Hpgds | NM_019455.4 | 0.0350 |
| Ltbr | NM_010736.3 | 0.0352 |
| Cul1 | NM_012042.3 | 0.0360 |
| Tardbp | NM_001003899.2 | 0.0362 |
| Grik2 | NM_010349.2 | 0.0365 |
| Esam | NM_027102.3 | 0.0366 |
| Pls1 | NM_001033210.3 | 0.0368 |
| Smpd4 | NM_001164610.1 | 0.0369 |
| Phf19 | NM_028716.4 | 0.0370 |
| Lrp1 | NM_008512.2 | 0.0373 |
| Pink1 | NM_026880.2 | 0.0373 |
| Ncf1 | NM_001286037.1 | 0.0379 |
| Mmp16 | NM_019724.3 | 0.0380 |
| Tnf | NM_013693.2 | 0.0380 |
| Arhgap44 | NM_001099288.1 | 0.0383 |
| Hdac7 | NM_019572.2 | 0.0387 |
| Cd34 | NM_001111059.1 | 0.0391 |
| Nrg1 | NM_178591.2 | 0.0396 |
| Gad1 | NM_008077.4 | 0.0397 |
| Akt3 | NM_011785.3 | 0.0397 |
| Nf1 | NM_010897.2 | 0.0404 |
| Cldn5 | NM_013805.4 | 0.0407 |
| Epha6 | NM_007938.2 | 0.0412 |
| Gnai1 | NM_010305.1 | 0.0413 |
| Idh1 | NM_010497.2 | 0.0413 |
| Fam104a | NM_138598.5 | 0.0414 |
| Optn | NM_181848.4 | 0.0416 |
| Gata2 | NM_008090.4 | 0.0417 |
| Comt | NM_007744.3 | 0.0418 |
| Sox10 | XM_128139.6 | 0.0431 |
| Taf4 | NM_001081092.1 | 0.0433 |
| Car2 | NM_009801.4 | 0.0435 |
| Mapk9 | NM_207692.1 | 0.0444 |
| Cdk2 | NM_016756.4 | 0.0447 |
| Nefl | NM_010910.1 | 0.0455 |
| Hdac6 | NM_010413.3 | 0.0459 |
| Calml | NM_009790.4 | 0.0465 |
| Cers6 | NM_172856.3 | 0.0467 |
| Nlrp3 | NM_145827.3 | 0.0474 |
| Nfe2l2 | NR_132727.1 | 0.0477 |
| Polr2h | NM_145632.2 | 0.0485 |
| S100b | NM_009115.3 | 0.0486 |
| Nr4a2 | NM_001139509.1 | 0.0487 |
| Tgfbr2 | NM_009371.2 | 0.0497 |
| Gss | NM_008180.1 | 0.0498 |
| Bid | NM_007544.3 | 0.0500 |

TABLE 10
From TTPB_0400_final document,
Differentially Expressed Genes-DIV11

| Gene Name | Accession # | Pval |
|---|---|---|
| C3 | XM_011246258.1 | 0.0000 |
| Nostrin | NM_181547.3 | 0.0000 |
| Igf1 | NM_010512.4 | 0.0000 |
| Fgf2 | NM_008006.2 | 0.0001 |
| Slc2a1 | NM_011400.3 | 0.0001 |
| Cntn4 | NM_001109751.1 | 0.0001 |
| Ppp2r5e | NM_012024.2 | 0.0002 |
| Igf1r | NM_010513.2 | 0.0003 |
| Cnr1 | NM_007726.3 | 0.0003 |
| Lsr | NM_001164184.1 | 0.0004 |
| Cxcl12 | NM_021704.3 | 0.0004 |
| Ccnd1 | NM_007631.1 | 0.0004 |
| Axin2 | NM_015732.4 | 0.0004 |
| Cntnap2 | NM_001004357.2 | 0.0005 |
| Plxnb3 | NM_019587.2 | 0.0005 |
| Gabra4 | NM_010251.2 | 0.0005 |
| Negr1 | NM_001039094.2 | 0.0006 |
| Cdk5r1 | NM_009871.2 | 0.0006 |
| Cacna1b | NM_001042528.1 | 0.0006 |
| Syt1 | NM_009306.2 | 0.0006 |
| Esam | NM_027102.3 | 0.0007 |
| Cdkn1a | NM_007669.4 | 0.0007 |
| Plcb1 | NM_019677.1 | 0.0007 |
| Nfl | NM_010897.2 | 0.0007 |
| Islr2 | NM_001161538.1 | 0.0007 |
| Atp8a2 | NM_015803.2 | 0.0008 |
| Grin2c | NM_010350.2 | 0.0008 |
| Pla2g4a | NM_008869.2 | 0.0008 |
| Cd44 | NM_009851.2 | 0.0009 |
| Xiap | NM_009688.2 | 0.0010 |
| Epha5 | NM_007937.3 | 0.0011 |
| Olfm3 | NM_153157.3 | 0.0011 |
| Mmp2 | NM_008610.2 | 0.0011 |
| Gata2 | NM_008090.4 | 0.0012 |
| Cp | NM_001042611.1 | 0.0013 |
| Slc32a1 | NM_009508.2 | 0.0013 |
| Dlg3 | NM_001177778.1 | 0.0014 |
| Grin2b | NM_008171.3 | 0.0014 |
| Clu | NM_013492.2 | 0.0014 |
| Cd9 | NM_007657.3 | 0.0014 |
| Pmp | NM_011170.2 | 0.0015 |
| Aars | NM_146217.4 | 0.0015 |
| Epha7 | NM_001122889.1 | 0.0016 |
| Parp1 | NM_007415.2 | 0.0016 |
| Ep300 | NM_177821.6 | 0.0016 |
| Cntn1 | NM_001159647.1 | 0.0016 |
| Tmem119 | NM_146162.2 | 0.0018 |
| Nrxn1 | NM_001346960.1 | 0.0018 |
| Cd68 | NM_009853.1 | 0.0018 |
| Itpr1 | NM_010585.2 | 0.0019 |
| Dagla | NM_198114.2 | 0.0019 |
| Hmox1 | NM_010442.2 | 0.0019 |
| Casp3 | NM_009810.2 | 0.0021 |
| P2ry12 | NM_027571.3 | 0.0021 |
| Amigo1 | NM_001004293.2 | 0.0021 |
| Gnai3 | NM_010306.2 | 0.0021 |
| Ptprn2 | NM_011215.2 | 0.0022 |
| Csf1r | NM_001037859.1 | 0.0022 |
| Gabrb2 | NM_008070.3 | 0.0022 |
| Gsr | NM_010344.4 | 0.0023 |
| Crebbp | NM_001025432.1 | 0.0023 |
| Lmna | NM_001002011.2 | 0.0026 |
| Scn1a | NM_018733.2 | 0.0027 |
| Chrnb2 | NM_009602.4 | 0.0027 |
| Calbl | NM_009788.4 | 0.0028 |
| Ryr2 | NM_023868.1 | 0.0029 |
| Tbpl1 | NM_011603.5 | 0.0029 |
| Cx3cl1 | NM_009142.3 | 0.0029 |
| Creb1 | NM_001037726.1 | 0.0029 |
| Lrrk2 | NM_025730.3 | 0.0029 |
| Fnl | NM_010233.1 | 0.0029 |
| Gnb5 | NM_138719.5 | 0.0030 |
| Mtor | NM_020009.2 | 0.0030 |
| Bax | NM_007527.3 | 0.0030 |
| Camk2g | NM_001039138.1 | 0.0030 |
| Cxcr4 | NM_009911.3 | 0.0030 |
| Rapgef2 | NM_001099624.2 | 0.0031 |
| Itga5 | NM_001314041.1 | 0.0032 |
| Trpm2 | NM_138301.2 | 0.0032 |
| Gusb | NM_010368.1 | 0.0033 |
| AI464131 | NM_001085515.2 | 0.0033 |
| Cers1 | NM_138647.3 | 0.0034 |
| Park2 | NM_016694.3 | 0.0034 |
| Pkn1 | NM_001199593.1 | 0.0035 |
| Ppt1 | NM_008917.3 | 0.0035 |
| Gabra1 | NM_010250.4 | 0.0035 |
| Frmpd4 | NM_001033330.2 | 0.0035 |
| Msn | NM_010833.2 | 0.0036 |
| Prpf31 | NM_001159714.1 | 0.0036 |
| Pla2g4b | XM_925095.2 | 0.0037 |
| Gria4 | NM_001113180.1 | 0.0038 |
| Flt1 | NM_010228.3 | 0.0038 |
| Cx3cr1 | NM_009987.3 | 0.0038 |
| Gabrb3 | NM_008071.3 | 0.0039 |
| Il1r1 | NM_001123382.1 | 0.0039 |
| Sorcs3 | NM_025696.3 | 0.0039 |
| Cadm3 | NM_053199.3 | 0.0041 |
| Grin1 | NM_008169.2 | 0.0041 |
| Arsa | NM_009713.4 | 0.0041 |
| Cd33 | NM_001111058.1 | 0.0042 |
| Il10ra | NM_008348.2 | 0.0043 |
| Ache | NM_009599.3 | 0.0043 |
| Grm8 | NM_008174.2 | 0.0044 |
| Gprasp1 | NM_001004359.2 | 0.0046 |
| Epha4 | NM_007936.3 | 0.0048 |
| Egf7 | NM_001164564.1 | 0.0048 |
| Cacnb2 | NM_023116.3 | 0.0048 |
| Gucy1b3 | NM_017469.4 | 0.0049 |
| Scn2a1 | NM_001099298.3 | 0.0049 |
| Stat3 | NM_213659.2 | 0.0049 |
| Nell2 | NM_016743.2 | 0.0050 |
| Tspo | NM_009775.4 | 0.0050 |
| Adra2a | NM_007417.4 | 0.0050 |
| Apaf1 | NM_009684.2 | 0.0050 |
| Mapkapk2 | NM_008551.1 | 0.0050 |
| Thy1 | NM_009382.3 | 0.0051 |
| Hif1a | NM_010431.2 | 0.0052 |
| Cds1 | NM_173370.3 | 0.0053 |
| Cacna1d | NM_028981.2 | 0.0053 |
| Hpgds | NM_019455.4 | 0.0055 |
| Eif2s1 | NM_026114.3 | 0.0055 |
| Chrna7 | NM_007390.3 | 0.0056 |
| Jun | NM_010591.2 | 0.0059 |
| Atcay | NM_178662.3 | 0.0063 |
| Cast | NM_009817.1 | 0.0063 |
| Spast | NM_016962.2 | 0.0063 |
| Ncam1 | NM_001113204.1 | 0.0063 |
| Pcsk2 | NM_008792.3 | 0.0064 |
| Ggt1 | NM_008116.2 | 0.0066 |
| Ran | NM_009391.3 | 0.0069 |
| Gria3 | NM_016886.3 | 0.0069 |
| Ccr5 | NM_009917.5 | 0.0069 |
| Plcb4 | NM_013829.2 | 0.0069 |
| Eng | NM_001146350.1 | 0.0070 |
| L1cam | NM_008478.3 | 0.0071 |
| Fgf12 | NM_001276419.1 | 0.0072 |
| Slc9a6 | NM_172780.3 | 0.0073 |
| Cdc40 | NM_027879.2 | 0.0074 |
| Unc13a | NM_001029873.2 | 0.0075 |
| Emp2 | NM_007929.2 | 0.0076 |
| Magee1 | NM_053201.3 | 0.0076 |
| Slc12a5 | NM_020333.2 | 0.0077 |
| Grm1 | NM_001114333.2 | 0.0077 |
| Sri | NM_001080974.2 | 0.0077 |
| Tnc | NM_011607.1 | 0.0079 |
| Atp6v0e | NM_025272.2 | 0.0080 |
| Cxcl16 | NM_023158.6 | 0.0080 |
| Vegfa | NM_001025250.3 | 0.0080 |
| Naglu | NM_013792.2 | 0.0080 |
| B4galt6 | NM_019737.2 | 0.0080 |
| Hdac1 | NM_008228.2 | 0.0082 |
| Nos1 | NM_008712.2 | 0.0084 |
| Dll4 | NM_019454.2 | 0.0089 |
| Gria2 | NM_001039195.1 | 0.0091 |
| Myc | NM_010849.4 | 0.0092 |
| Atxn7 | NM_139227.4 | 0.0094 |
| Shh | NM_009170.3 | 0.0096 |
| Cadps | NM_001042617.1 | 0.0096 |
| Lsm2 | NM_001110101.2 | 0.0096 |
| Adcy8 | NM_009623.2 | 0.0098 |
| Sorl1 | NM_011436.3 | 0.0099 |
| Camk2b | NM_001174053.1 | 0.0099 |
| Nefh | NM_010904.3 | 0.0099 |
| Sf3b2 | NM_030109.2 | 0.0100 |
| Mapk10 | NM_001081567.1 | 0.0100 |
| Lama2 | NM_008481.2 | 0.0100 |
| Gls | NM_001113383.1 | 0.0101 |
| Gaa | NM_008064.3 | 0.0102 |
| Apoe | NM_001305844.1 | 0.0102 |
| Lox | NM_010728.2 | 0.0104 |
| Acvrl1 | NM_009612.2 | 0.0105 |
| Entpd2 | NM_009849.2 | 0.0105 |
| Kif3a | NM_008443.3 | 0.0106 |
| Pfn1 | NM_011072.4 | 0.0106 |
| Tenm2 | NM_011856.3 | 0.0107 |
| Htt | NM_010414.1 | 0.0107 |
| Gabrg2 | NM_177408.5 | 0.0108 |
| Dgke | NM_019505.3 | 0.0108 |
| Pik3r1 | NM_001024955.1 | 0.0109 |
| Tnfrsf1a | NM_011609.2 | 0.0109 |
| Myh10 | NM_175260.2 | 0.0110 |
| Slc8a1 | NM_011406.2 | 0.0116 |
| Insr | NM_010568.2 | 0.0116 |
| Ctns | NM_031251.4 | 0.0118 |
| Gad1 | NM_008077.4 | 0.0119 |
| Ppp3ca | NM_008913.4 | 0.0119 |
| Camk4 | NM_009793.3 | 0.0119 |
| Sncb | NM_033610.2 | 0.0120 |
| Psen1 | NM_008943.2 | 0.0120 |
| Nptn | NM_009145.2 | 0.0122 |
| Sf3a2 | NM_013651.4 | 0.0124 |
| Grin2a | NM_008170.2 | 0.0125 |
| Nfe2l2 | NR_132727.1 | 0.0125 |
| Atg5 | NM_053069.5 | 0.0126 |
| Fmr1 | NM_008031.2 | 0.0127 |
| Trp53 | NM_011640.1 | 0.0128 |
| Txnll | NM_016792.2 | 0.0130 |
| Grm2 | NM_001160353.1 | 0.0131 |
| Sp100 | NM_013673.3 | 0.0131 |
| Cacna1a | NM_007578.3 | 0.0131 |
| Polr2k | NM_001039368.1 | 0.0131 |
| Inpp4a | NM_172971.2 | 0.0132 |
| Csf1 | NM_001113530.1 | 0.0132 |
| Efna5 | NM_207654.2 | 0.0133 |
| Mmp19 | NM_021412.2 | 0.0135 |
| Dlat | NM_145614.4 | 0.0138 |
| Drd1 | NM_010076.3 | 0.0139 |
| Kcnb1 | NM_008420.3 | 0.0140 |
| Npy | NM_023456.2 | 0.0141 |
| Slc4a10 | NM_001242380.1 | 0.0142 |
| Ccdc127 | NM_024201.3 | 0.0142 |
| Sgpl1 | NM_009163.3 | 0.0144 |
| Gng2 | NM_010315.4 | 0.0144 |
| Tcirg1 | NM_001136091.1 | 0.0147 |
| Lsm7 | NM_025349.2 | 0.0149 |
| Atp2b3 | NM_177236.3 | 0.0149 |
| Mmp12 | NM_008605.3 | 0.0149 |
| Mapk1 | NM_011949.3 | 0.0152 |
| Ngf | NM_001112698.1 | 0.0153 |
| Trim28 | NM_011588.3 | 0.0153 |
| Psen2 | NM_001128605.1 | 0.0154 |
| Vip | NM_011702.2 | 0.0161 |
| Cldn5 | NM_013805.4 | 0.0162 |
| Cdk5rap3 | NM_030248.1 | 0.0165 |
| Mto1 | NM_026658.2 | 0.0165 |
| Epha3 | NM_010140.3 | 0.0166 |
| Dgkb | NM_178681.4 | 0.0169 |
| Casp1 | NM_009807.2 | 0.0170 |
| Ppp3cc | NM_008915.2 | 0.0171 |
| Pmp22 | NM_008885.2 | 0.0172 |
| Sp1 | NM_013672.2 | 0.0173 |
| Tnfrsf12a | NM_001161746.1 | 0.0178 |
| Htr1a | NM_008308.4 | 0.0178 |
| Slc1a1 | NM_009199.2 | 0.0180 |
| Nlrp3 | NM_145827.3 | 0.0180 |
| Efna1 | NM_010107.4 | 0.0182 |
| Sirt1 | NM_019812.2 | 0.0187 |
| Rhoa | NM_016802.4 | 0.0190 |
| Gfap | NM_001131020.1 | 0.0191 |
| Src | NM_001025395.2 | 0.0191 |
| Raf1 | NM_029780.3 | 0.0191 |
| Vep | NM_009503.3 | 0.0194 |
| Angpt2 | NM_007426.3 | 0.0195 |
| Sirt7 | NM_153056.2 | 0.0196 |
| Lrrc4 | NM_138682.2 | 0.0196 |
| Nqo1 | NM_008706.5 | 0.0197 |
| Mfn2 | XM_006535920.1 | 0.0197 |
| Pgk1 | NM_008828.2 | 0.0198 |
| Prkcsh | NM_008925.1 | 0.0199 |
| I16ra | NM_010559.2 | 0.0201 |
| Itpr2 | NM_010586.1 | 0.0202 |
| Tie1 | NM_011587.2 | 0.0204 |
| Rdx | NM_001104617.1 | 0.0206 |
| Adcy9 | NM_009624.1 | 0.0206 |
| Bcas1 | NM_029815.2 | 0.0207 |
| Atxn3 | NM_001167914.1 | 0.0212 |
| Ppp3cb | NM_008914.1 | 0.0223 |
| Ang | NM_007447.2 | 0.0224 |
| Col4a2 | NM_009932.3 | 0.0225 |
| Map2 | NM_008632.2 | 0.0227 |
| Xbp1 | NM_013842.2 | 0.0229 |
| Grn | NM_008175.3 | 0.0233 |
| Cacna1c | NM_001159535.1 | 0.0234 |
| Adrb2 | NM_007420.2 | 0.0235 |
| Sucla2 | NM_011506.1 | 0.0235 |
| Mapk9 | NM_207692.1 | 0.0240 |
| Ipcef1 | NM_001033391.2 | 0.0243 |
| Tnr | NM_022312.3 | 0.0244 |
| Snea | NM_009221.2 | 0.0244 |
| Epha6 | NM_007938.2 | 0.0246 |
| Cnot10 | NM_153585.5 | 0.0246 |
| Mapk8 | NM_016700.3 | 0.0246 |
| Gad2 | NM_008078.2 | 0.0248 |
| Cyp4x1 | NM_001003947.1 | 0.0249 |
| Tbp | NM_013684.3 | 0.0251 |
| Synj1 | NM_001045515.1 | 0.0251 |
| Serpinb6a | NM_001164117.1 | 0.0252 |
| Trem2 | NM_031254.2 | 0.0253 |
| Ptgs2 | NM_011198.3 | 0.0254 |
| Fcrls | NM_030707.3 | 0.0257 |
| Mgmt | NM_008598.2 | 0.0260 |
| Plxncl | NM_018797.2 | 0.0266 |
| Itgam | NM_008401.2 | 0.0266 |
| I14ra | NM_001008700.3 | 0.0267 |
| Mmp24 | NM_010808.3 | 0.0267 |
| Akt3 | NM_011785.3 | 0.0269 |
| Gsk3b | NM_019827.3 | 0.0272 |
| Icam1 | NM_010493.2 | 0.0274 |
| Mmp14 | NM_008608.3 | 0.0276 |
| Ugcg | NM_011673.3 | 0.0279 |
| Cdc27 | NM_145436.2 | 0.0281 |
| Fas | NM_007987.2 | 0.0282 |
| Tardbp | NM_001003899.2 | 0.0283 |
| Olig2 | NM_016967.2 | 0.0284 |
| Lamp1 | NM_010684.2 | 0.0287 |
| Atp6v1d | NM_023721.2 | 0.0288 |
| Hcn1 | NM_010408.3 | 0.0288 |
| Rac1 | NM_009007.2 | 0.0288 |
| Pde4d | NM_011056.3 | 0.0292 |
| Ptprr | NM_011217.2 | 0.0296 |
| Plekho2 | NM_153119.2 | 0.0301 |
| Dnml1 | NM_001025947.1 | 0.0304 |
| Jam3 | NM_023277.4 | 0.0305 |
| Hexb | NM_010422.2 | 0.0305 |
| Nsf | NM_008740.2 | 0.0309 |
| Htr5a | NM_008314.2 | 0.0310 |
| Ap2b1 | NM_001035854.2 | 0.0310 |
| Prkaa2 | NM_178143.1 | 0.0311 |
| Cdk2 | NM_016756.4 | 0.0311 |
| Polr2j | NM_011293.2 | 0.0316 |
| Nos3 | NM_008713.4 | 0.0319 |
| Homer1 | NM_147176.2 | 0.0319 |
| Ap4s1 | NM_021710.3 | 0.0319 |
| Chmp2b | NM_026879.2 | 0.0320 |
| Ikbkb | NM_010546.2 | 0.0321 |
| Nrg1 | NM_178591.2 | 0.0323 |
| Syt13 | NM_030725.4 | 0.0324 |
| Fxn | NM_008044.2 | 0.0326 |
| Grik2 | NM_010349.2 | 0.0331 |
| Galc | NM_008079.3 | 0.0331 |
| Srsf4 | NM_020587.2 | 0.0339 |
| Ap3m2 | NM_029505.3 | 0.0341 |
| Cd34 | NM_001111059.1 | 0.0346 |
| Rab3a | NM_009001.6 | 0.0347 |
| Ube2n | NM_080560.3 | 0.0347 |
| Ppp2ca | NM_019411.4 | 0.0350 |
| Rad23b | NM_009011.4 | 0.0357 |
| Amph | NM_175007.1 | 0.0358 |
| Tnf | NM_013693.2 | 0.0361 |
| Erg | NM_133659.2 | 0.0361 |
| Arhgap44 | NM_001099288.1 | 0.0369 |
| Marco | NM_010766.2 | 0.0370 |
| Cybb | NM_007807.2 | 0.0371 |
| Zfp24 | NM_021559.2 | 0.0375 |
| Ccl12 | NM_011331.2 | 0.0376 |
| Ring1 | NM_009066.3 | 0.0376 |
| Fgf14 | NM_207667.3 | 0.0396 |
| Nes | NM_016701.3 | 0.0399 |
| Gpdl1 | NM_175380.5 | 0.0404 |
| Cntnap1 | NM_016782.2 | 0.0409 |
| Nme5 | NM_080637.3 | 0.0411 |
| Grm5 | NM_001143834.1 | 0.0415 |
| Gnaq | NM_008139.5 | 0.0415 |
| Lamb2 | NM_008483.3 | 0.0416 |
| Map2k1 | NM_008927.3 | 0.0418 |
| Pnkd | NM_019999.2 | 0.0418 |
| Optn | NM_181848.4 | 0.0419 |
| Cln8 | NM_012000.3 | 0.0423 |
| Cers4 | NM_026058.4 | 0.0425 |
| Atp6v0c | NM_009729.3 | 0.0425 |
| Ina | NM_146100.4 | 0.0426 |
| Gnai1 | NM_010305.1 | 0.0427 |
| Phf19 | NM_028716.4 | 0.0431 |
| Ltbr | NM_010736.3 | 0.0435 |
| Ces | XM_006531645.1 | 0.0440 |
| Park7 | NM_020569.3 | 0.0442 |
| Rasgrp1 | NM_011246.2 | 0.0443 |
| Sox10 | XM_128139.6 | 0.0443 |
| Cers2 | NM_029789.1 | 0.0448 |
| Atp6v0e2 | NM_133764.3 | 0.0455 |
| Pgam1 | NM_023418.2 | 0.0456 |
| Osmr | NM_011019.3 | 0.0460 |
| Atp7a | NM_001109757.2 | 0.0465 |
| Fyn | NM_008054.2 | 0.0470 |
| Cul1 | NM_012042.3 | 0.0471 |
| Man2b1 | NM_010764.2 | 0.0472 |
| Ryr1 | NM_009109.1 | 0.0478 |
| Notch3 | NM_008716.2 | 0.0478 |
| Gnao1 | NM_010308.3 | 0.0489 |
| Oxr1 | NM_001130163.1 | 0.0491 |
| Dld | NM_007861.4 | 0.0494 |
| Cycs | NM_007808.4 | 0.0496 |

TABLE 11
From TTPB_0400_final document,
Differentially Expressed Genes-DIV14

| Gene Name | Accession # | Pval |
|---|---|---|
| Slc2a1 | NM_011400.3 | 0.0000 |
| Cd44 | NM_009851.2 | 0.0000 |
| Nf1 | NM_010897.2 | 0.0000 |
| Cnr1 | NM_007726.3 | 0.0000 |
| Cp | NM_001042611.1 | 0.0001 |
| Abat | NM_001170978.1 | 0.0001 |
| Osmr | NM_011019.3 | 0.0001 |
| Adrb2 | NM_007420.2 | 0.0001 |
| Flt1 | NM_010228.3 | 0.0001 |
| Syt1 | NM_009306.2 | 0.0001 |
| Chrna7 | NM_007390.3 | 0.0001 |
| Mmp2 | NM_008610.2 | 0.0002 |
| Ccnd1 | NM_007631.1 | 0.0002 |
| Serpinb6a | NM_001164117.1 | 0.0002 |
| Cntn4 | NM_001109751.1 | 0.0002 |
| Slc4a10 | NM_001242380.1 | 0.0002 |
| Epha5 | NM_007937.3 | 0.0002 |
| PlC12 | NM_013880.3 | 0.0002 |
| Hdac1 | NM_008228.2 | 0.0002 |
| P2ry12 | NM_027571.3 | 0.0003 |
| Plcb1 | NM_019677.1 | 0.0003 |
| Atm | NM_009730.2 | 0.0003 |
| Synj1 | NM_001045515.1 | 0.0004 |
| Ptprn2 | NM_011215.2 | 0.0004 |
| Lox | NM_010728.2 | 0.0004 |
| Kcnb1 | NM_008420.3 | 0.0004 |
| App | NM_007471.2 | 0.0004 |
| Tada2b | NM_001170454.1 | 0.0005 |
| Thy1 | NM_009382.3 | 0.0005 |
| Gprasp1 | NM_001004359.2 | 0.0005 |
| Rapgef2 | NM_001099624.2 | 0.0005 |
| Csflr | NM_001037859.1 | 0.0005 |
| Illr1 | NM_001123382.1 | 0.0006 |
| Sp100 | NM_013673.3 | 0.0006 |
| Nes | NM_016701.3 | 0.0007 |
| Glrb | NM_010298.5 | 0.0007 |
| Dgkb | NM_178681.4 | 0.0007 |
| Slc1a2 | NM_001077514.3 | 0.0008 |
| Rasgrp1 | NM_011246.2 | 0.0008 |
| Atp8a2 | NM_015803.2 | 0.0008 |
| Dlg3 | NM_001177778.1 | 0.0008 |
| Camk2g | NM_001039138.1 | 0.0008 |
| Cdkn1a | NM_007669.4 | 0.0009 |
| Pfn1 | NM_011072.4 | 0.0009 |
| Olig2 | NM_016967.2 | 0.0009 |
| Scn1a | NM_018733.2 | 0.0010 |
| Apaf1 | NM_009684.2 | 0.0010 |
| Casp7 | NM_007611.2 | 0.0011 |
| Igf1r | NM_010513.2 | 0.0011 |
| Gtf2h1 | NM_008186.4 | 0.0011 |
| Grin2b | NM_008171.3 | 0.0012 |
| Gstp1 | NM_013541.1 | 0.0012 |
| Cxcl12 | NM_021704.3 | 0.0012 |
| Gata2 | NM_008090.4 | 0.0012 |
| Islr2 | NM_001161538.1 | 0.0012 |
| Prkce | NM_011104.2 | 0.0012 |
| Cadm3 | NM_053199.3 | 0.0013 |
| Syt7 | NM_018801.3 | 0.0014 |
| Slc12a5 | NM_020333.2 | 0.0015 |
| Hmox1 | NM_010442.2 | 0.0015 |
| Insr | NM_010568.2 | 0.0015 |
| Trpm2 | NM_138301.2 | 0.0015 |
| Ncam1 | NM_001113204.1 | 0.0016 |
| Nrxn1 | NM_001346960.1 | 0.0016 |
| Tmem119 | NM_146162.2 | 0.0016 |
| Myc | NM_010849.4 | 0.0017 |
| Negr1 | NM_001039094.2 | 0.0017 |
| C3 | XM_011246258.1 | 0.0018 |
| Prkacb | NM_011100.3 | 0.0019 |
| Lsr | NM_001164184.1 | 0.0019 |
| Vegfa | NM_001025250.3 | 0.0019 |
| L1cam | NM_008478.3 | 0.0020 |
| Cul1 | NM_012042.3 | 0.0020 |
| Casp1 | NM_009807.2 | 0.0021 |
| Atp2b3 | NM_177236.3 | 0.0022 |
| Grm8 | NM_008174.2 | 0.0022 |
| Epha7 | NM_001122889.1 | 0.0022 |
| Gabra1 | NM_010250.4 | 0.0022 |
| Ppp3ca | NM_008913.4 | 0.0023 |
| Grin2c | NM_010350.2 | 0.0023 |
| Comt | NM_007744.3 | 0.0024 |
| Cacna1b | NM_001042528.1 | 0.0025 |
| Gucy1b3 | NM_017469.4 | 0.0026 |
| Mmp14 | NM_008608.3 | 0.0026 |
| Msn | NM_010833.2 | 0.0026 |
| Mmp12 | NM_008605.3 | 0.0027 |
| Cycs | NM_007808.4 | 0.0027 |
| Dll4 | NM_019454.2 | 0.0028 |
| P2rx4 | NM_011026.2 | 0.0028 |
| Nefh | NM_010904.3 | 0.0028 |
| Ryr2 | NM_023868.1 | 0.0029 |
| Cast | NM_009817.1 | 0.0029 |
| Sod2 | NM_013671.3 | 0.0030 |
| Slc11a1 | NM_013612.2 | 0.0032 |
| Mmrn2 | NM_153127.3 | 0.0032 |
| Cers1 | NM_138647.3 | 0.0033 |
| Atg5 | NM_053069.5 | 0.0033 |
| Cxcl10 | NM_021274.1 | 0.0034 |
| Camk2b | NM_001174053.1 | 0.0035 |
| Vcp | NM_009503.3 | 0.0035 |
| Bid | NM_007544.3 | 0.0035 |
| Sncb | NM_033610.2 | 0.0036 |
| Pla2g4b | XM_925095.2 | 0.0037 |
| Egfl7 | NM_001164564.1 | 0.0038 |
| Cxcl16 | NM_023158.6 | 0.0038 |
| Tenm2 | NM_011856.3 | 0.0039 |
| Prkcb | NM_008855.2 | 0.0039 |
| Scn2a1 | NM_001099298.3 | 0.0039 |
| Ptprr | NM_011217.2 | 0.0040 |
| Cntn1 | NM_001159647.1 | 0.0041 |
| Gabrb2 | NM_008070.3 | 0.0041 |
| Grin1 | NM_008169.2 | 0.0041 |
| Gsr | NM_010344.4 | 0.0042 |
| Csf1 | NM_001113530.1 | 0.0042 |
| Mag | NM_010758.2 | 0.0042 |
| Esam | NM_027102.3 | 0.0043 |
| Itga5 | NM_001314041.1 | 0.0043 |
| Icam2 | NM_010494.1 | 0.0044 |
| Gls | NM_001113383.1 | 0.0045 |
| Ppp3cc | NM_008915.2 | 0.0045 |
| Nsf | NM_008740.2 | 0.0045 |
| Taz | NM_181516.5 | 0.0046 |
| Prkcg | NM_011102.3 | 0.0047 |
| Ap2a2 | NM_007459.3 | 0.0048 |
| Ap4s1 | NM_021710.3 | 0.0048 |
| Trp53 | NM_011640.1 | 0.0049 |
| Ipcef1 | NM_001033391.2 | 0.0049 |
| Lmna | NM_001002011.2 | 0.0051 |
| Tnfrsf12a | NM_001161746.1 | 0.0051 |
| Cybb | NM_007807.2 | 0.0051 |
| Lama2 | NM_008481.2 | 0.0051 |
| Gad1 | NM_008077.4 | 0.0052 |
| Sorl1 | NM_011436.3 | 0.0053 |
| Cnksr2 | NM_177751.2 | 0.0053 |
| Magee1 | NM_053201.3 | 0.0054 |
| Ache | NM_009599.3 | 0.0055 |
| Gria4 | NM_001113180.1 | 0.0056 |
| Tspo | NM_009775.4 | 0.0056 |
| Cadps | NM_001042617.1 | 0.0057 |
| Cacnb2 | NM_023116.3 | 0.0057 |
| Tnfrsf1a | NM_011609.2 | 0.0057 |
| I16ra | NM_010559.2 | 0.0059 |
| Cxcr4 | NM_009911.3 | 0.0059 |
| Slc8a1 | NM_011406.2 | 0.0060 |
| Cd68 | NM_009853.1 | 0.0060 |
| Chl1 | NM_007697.2 | 0.0060 |
| Epha4 | NM_007936.3 | 0.0061 |
| Cd9 | NM_007657.3 | 0.0063 |
| Efna5 | NM_207654.2 | 0.0065 |
| Syt13 | NM_030725.4 | 0.0065 |
| Cntnap1 | NM_016782.2 | 0.0065 |
| Tcirg1 | NM_001136091.1 | 0.0066 |
| Mapk10 | NM_001081567.1 | 0.0066 |
| Atcay | NM_178662.3 | 0.0066 |
| Pak1 | NM_011035.2 | 0.0067 |
| Cers2 | NM_029789.1 | 0.0067 |
| Hcn1 | NM_010408.3 | 0.0067 |
| Ugeg | NM_011673.3 | 0.0068 |
| Stx1b | NM_024414.2 | 0.0069 |
| Fgf14 | NM_207667.3 | 0.0069 |
| Pcsk2 | NM_008792.3 | 0.0070 |
| AI464131 | NM_001085515.2 | 0.0072 |
| Nfe2l2 | NR_132727.1 | 0.0072 |
| Calb1 | NM_009788.4 | 0.0072 |
| Eng | NM_001146350.1 | 0.0073 |
| Hras | NM_001130443.1 | 0.0074 |
| Gad2 | NM_008078.2 | 0.0075 |
| Frmpd4 | NM_001033330.2 | 0.0075 |
| Rab3a | NM_009001.6 | 0.0077 |
| Grm1 | NM_001114333.2 | 0.0077 |
| Ltbr | NM_010736.3 | 0.0077 |
| Gpdl1 | NM_175380.5 | 0.0078 |
| Cacna1a | NM_007578.3 | 0.0080 |
| Gria2 | NM_001039195.1 | 0.0081 |
| Cntnap2 | NM_001004357.2 | 0.0082 |
| Fgf2 | NM_008006.2 | 0.0084 |
| Nos1 | NM_008712.2 | 0.0084 |
| Epha6 | NM_007938.2 | 0.0085 |
| Jun | NM_010591.2 | 0.0086 |
| Nlrp3 | NM_145827.3 | 0.0087 |
| Unc13a | NM_001029873.2 | 0.0092 |
| Map2k1 | NM_008927.3 | 0.0093 |
| Ugt8a | NM_011674.4 | 0.0093 |
| Emp2 | NM_007929.2 | 0.0093 |
| Atp6v0e2 | NM_133764.3 | 0.0095 |
| Shh | NM_009170.3 | 0.0096 |
| Nell2 | NM_016743.2 | 0.0096 |
| Nwd1 | NM_176940.5 | 0.0097 |
| Bax | NM_007527.3 | 0.0098 |
| Tnc | NM_011607.1 | 0.0098 |
| Il10ra | NM_008348.2 | 0.0100 |
| Cdk5r1 | NM_009871.2 | 0.0103 |
| Cds1 | NM_173370.3 | 0.0103 |
| Flt4 | NM_008029.3 | 0.0104 |
| Ina | NM_146100.4 | 0.0105 |
| Ube2k | NM_016786.3 | 0.0105 |
| Camk4 | NM_009793.3 | 0.0106 |
| Gria3 | NM_016886.3 | 0.0107 |
| Gabrg2 | NM_177408.5 | 0.0108 |
| Mfn2 | XM_006535920.1 | 0.0111 |
| Dlgap1 | NM_027712.3 | 0.0113 |
| Ggt1 | NM_008116.2 | 0.0115 |
| Atp6v1g2 | NM_023179.3 | 0.0115 |
| Vip | NM_011702.2 | 0.0117 |
| Ep300 | NM_177821.6 | 0.0120 |
| Akt3 | NM_011785.3 | 0.0120 |
| Amigol | NM_001004293.2 | 0.0121 |
| Stat3 | NM_213659.2 | 0.0121 |
| Sgpll | NM_009163.3 | 0.0121 |
| Grm2 | NM_001160353.1 | 0.0124 |
| Grin2a | NM_008170.2 | 0.0125 |
| Map2 | NM_008632.2 | 0.0125 |
| Hspb1 | NM_013560.2 | 0.0126 |
| Ptgs2 | NM_011198.3 | 0.0128 |
| Ddc | NM_016672.4 | 0.0128 |
| Arsa | NM_009713.4 | 0.0130 |
| Aars | NM_146217.4 | 0.0130 |
| Drd1 | NM_010076.3 | 0.0132 |
| Nqo1 | NM_008706.5 | 0.0133 |
| Inpp4a | NM_172971.2 | 0.0137 |
| Car2 | NM_009801.4 | 0.0138 |
| Dgke | NM_019505.3 | 0.0138 |
| Stx1a | NM_016801.3 | 0.0139 |
| Ap3m2 | NM_029505.3 | 0.0140 |
| Tbp | NM_013684.3 | 0.0140 |
| Snap91 | NM_001277986.1 | 0.0140 |
| Slc17a6 | NM_080853.3 | 0.0141 |
| Dag1a | NM_198114.2 | 0.0141 |
| Pmp22 | NM_008885.2 | 0.0142 |
| Trim37 | NM_197987.2 | 0.0142 |
| Notch3 | NM_008716.2 | 0.0143 |
| Dlat | NM_145614.4 | 0.0145 |
| Mmp19 | NM_021412.2 | 0.0145 |
| Tnfrsf1b | NM_011610.3 | 0.0145 |
| Nostrin | NM_181547.3 | 0.0147 |
| Plxnb3 | NM_019587.2 | 0.0147 |
| Ube2n | NM_080560.3 | 0.0148 |
| Gss | NM_008180.1 | 0.0148 |
| Myct1 | NM_026793.2 | 0.0149 |
| Itpr2 | NM_010586.1 | 0.0149 |
| Aldh1l1 | NM_027406.1 | 0.0150 |
| Cx3cl1 | NM_009142.3 | 0.0151 |
| Myrf | NM_001033481.1 | 0.0153 |
| Cldn5 | NM_013805.4 | 0.0154 |
| Prkcq | NM_008859.2 | 0.0154 |
| Igf1 | NM_010512.4 | 0.0156 |
| Xab2 | NM_026156.2 | 0.0157 |
| Gnao1 | NM_010308.3 | 0.0157 |
| Casp3 | NM_009810.2 | 0.0160 |
| Gtf2h3 | NM_181410.3 | 0.0162 |
| Mog | NM_010814.2 | 0.0163 |
| Gal3st1 | NM_001177691.1 | 0.0165 |
| Epha3 | NM_010140.3 | 0.0166 |
| Gsk3b | NM_019827.3 | 0.0167 |
| Dlg4 | NM_001109752.1 | 0.0168 |
| Slc32a1 | NM_009508.2 | 0.0171 |
| Chmb2 | NM_009602.4 | 0.0173 |
| Eif2s1 | NM_026114.3 | 0.0173 |
| Opa1 | NM_001199177.1 | 0.0175 |
| Gaa | NM_008064.3 | 0.0176 |
| Htr1a | NM_008308.4 | 0.0178 |
| Cacna1d | NM_028981.2 | 0.0179 |
| Gnai1 | NM_010305.1 | 0.0182 |
| Src | NM_001025395.2 | 0.0182 |
| Kras | NM_021284.5 | 0.0183 |
| Ppp3cb | NM_008914.1 | 0.0184 |
| Grik2 | NM_010349.2 | 0.0186 |
| Bace1 | NM_011792.4 | 0.0189 |
| Mapk8 | NM_016700.3 | 0.0196 |
| Fas | NM_007987.2 | 0.0199 |
| Calm1 | NM_009790.4 | 0.0199 |
| Acvrl1 | NM_009612.2 | 0.0200 |
| Sptbn2 | NM_021287.1 | 0.0200 |
| Rab3c | NM_023852.5 | 0.0200 |
| Stab1 | NM_138672.2 | 0.0203 |
| Gnaq | NM_008139.5 | 0.0203 |
| Tie1 | NM_011587.2 | 0.0207 |
| Gria1 | NM_001252403.1 | 0.0208 |
| Gusb | NM_010368.1 | 0.0212 |
| Atxn2 | NM_009125.2 | 0.0212 |
| Ccr5 | NM_009917.5 | 0.0212 |
| Cx3cr1 | NM_009987.3 | 0.0213 |
| Mapkapk2 | NM_008551.1 | 0.0213 |
| Cck | NM_031161.2 | 0.0218 |
| Sirt7 | NM_153056.2 | 0.0226 |
| Cd34 | NM_001111059.1 | 0.0229 |
| Fgf12 | NM_001276419.1 | 0.0231 |
| Pls1 | NM_001033210.3 | 0.0233 |
| Polr2k | NM_001039368.1 | 0.0234 |
| Pdpk1 | NM_001080773.2 | 0.0234 |
| Mbp | NM_010777.3 | 0.0235 |
| Polr2h | NM_145632.2 | 0.0236 |
| Cln8 | NM_012000.3 | 0.0241 |
| Cdk2 | NM_016756.4 | 0.0248 |
| Slc9a6 | NM_172780.3 | 0.0249 |
| Cyp4x1 | NM_001003947.1 | 0.0249 |
| Ppp2r5e | NM_012024.2 | 0.0261 |
| Trem2 | NM_031254.2 | 0.0262 |
| Ap2b1 | NM_001035854.2 | 0.0269 |
| Snca | NM_009221.2 | 0.0275 |
| Bcas1 | NM_029815.2 | 0.0276 |
| Ran | NM_009391.3 | 0.0279 |
| Ptdss2 | NM_013782.4 | 0.0282 |
| Lamb2 | NM_008483.3 | 0.0283 |
| Tnf | NM_013693.2 | 0.0284 |
| Rit2 | NM_009065.2 | 0.0286 |
| Ccl12 | NM_011331.2 | 0.0286 |
| Atxn7 | NM_139227.4 | 0.0294 |
| Dnml1 | NM_001025947.1 | 0.0299 |
| Parp1 | NM_007415.2 | 0.0299 |
| Crebbp | NM_001025432.1 | 0.0301 |
| Itgam | NM_008401.2 | 0.0302 |
| Psen1 | NM_008943.2 | 0.0303 |
| Lars | NM_134137.2 | 0.0304 |
| Ring1 | NM_009066.3 | 0.0305 |
| Htr5a | NM_008314.2 | 0.0310 |
| Nos3 | NM_008713.4 | 0.0319 |
| Pecam1 | NM_008816.2 | 0.0320 |
| Tardbp | NM_001003899.2 | 0.0322 |
| Amph | NM_175007.1 | 0.0322 |
| Ddit3 | NM_007837.3 | 0.0325 |
| Gsn | NM_146120.3 | 0.0327 |
| Nmnat2 | NM_175460.3 | 0.0337 |
| Icam1 | NM_010493.2 | 0.0339 |
| Rras | NM_009101.2 | 0.0340 |
| Atp13a2 | NM_029097.2 | 0.0341 |
| Col4a1 | NM_009931.2 | 0.0341 |
| Gabrb3 | NM_008071.3 | 0.0343 |
| Prkaca | NM_008854.3 | 0.0349 |
| Neo1 | NM_001042752.1 | 0.0352 |
| Nptn | NM_009145.2 | 0.0355 |
| Tbr1 | NM_009322.3 | 0.0360 |
| Erg | NM_133659.2 | 0.0361 |
| Grm5 | NM_001143834.1 | 0.0363 |
| Gnb5 | NM_138719.5 | 0.0365 |
| Itpr1 | NM_010585.2 | 0.0365 |
| Plcb4 | NM_013829.2 | 0.0369 |
| Ikbkb | NM_010546.2 | 0.0370 |
| Gabra4 | NM_010251.2 | 0.0375 |
| Notch4 | NM_010929.2 | 0.0378 |
| Sf3a2 | NM_013651.4 | 0.0379 |
| Wfs1 | NM_011716.2 | 0.0390 |
| Gba | NM_001077411.1 | 0.0395 |
| Kcnj10 | NM_001039484.1 | 0.0396 |
| Axin2 | NM_015732.4 | 0.0400 |
| Homer1 | NM_147176.2 | 0.0401 |
| Clu | NM_013492.2 | 0.0410 |
| Pde1b | NM_008800.1 | 0.0416 |
| Nts | NM_024435.2 | 0.0417 |
| Mmp24 | NM_010808.3 | 0.0422 |
| Txnl1 | NM_016792.2 | 0.0424 |
| Fus | NM_139149.2 | 0.0426 |
| B4galt6 | NM_019737.2 | 0.0426 |
| Egr1 | NM_007913.5 | 0.0426 |
| Atp6v0e | NM_025272.2 | 0.0427 |
| Adcy9 | NM_009624.1 | 0.0435 |
| Optn | NM_181848.4 | 0.0435 |
| Prnp | NM_011170.2 | 0.0438 |
| Sox9 | NM_011448.4 | 0.0439 |
| Gnptg | NM_172529.3 | 0.0440 |
| Cul2 | NM_029402.3 | 0.0451 |
| Lrrk2 | NM_025730.3 | 0.0456 |
| Mtor | NM_020009.2 | 0.0462 |
| Erlec1 | NM_025745.3 | 0.0472 |
| Ryr1 | NM_009109.1 | 0.0478 |
| Gfap | NM_001131020.1 | 0.0479 |

TABLE 12
From TTPB_0400_final document, Differentially Expressed Genes - GProfiler Results - DIV01

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Human cytomegalovirus infection | KEGG: 05163 | 1.88E−24 | 23.72543 | 242 | 289 | 51 | 8258 |
| KEGG | Long-term potentiation | KEGG: 04720 | 5.64E−23 | 22.24889 | 62 | 289 | 28 | 8258 |
| KEGG | MAPK signaling pathway | KEGG: 04010 | 4.59E−20 | 19.33812 | 286 | 289 | 50 | 8258 |
| KEGG | Glutamatergic synapse | KEGG: 04724 | 4.08E−19 | 18.3895 | 112 | 289 | 32 | 8258 |
| KEGG | Dopaminergic synapse | KEGG: 04728 | 2.53E−18 | 17.59637 | 127 | 289 | 33 | 8258 |
| KEGG | Oxytocin signaling pathway | KEGG: 04921 | 3.63E−17 | 16.43964 | 147 | 289 | 34 | 8258 |
| KEGG | Kaposi sarcoma-associated | KEGG: 05167 | 1.18E−16 | 15.92691 | 205 | 289 | 39 | 8258 |
| | herpesvirus infection | | | | | | | |
| KEGG | GnRH signaling pathway | KEGG: 04912 | 9.58E−16 | 15.01846 | 88 | 289 | 26 | 8258 |
| KEGG | Rapt signaling pathway | KEGG: 04015 | 1.72E−15 | 14.76553 | 209 | 289 | 38 | 8258 |
| KEGG | Relaxin signaling pathway | KEGG: 04926 | 2.70E−15 | 14.56817 | 127 | 289 | 30 | 8258 |
| KEGG | Amphetamine addiction | KEGG: 05031 | 9.04E−15 | 14.04391 | 64 | 289 | 22 | 8258 |
| KEGG | Apoptosis | KEGG: 04210 | 1.67E−14 | 13.77651 | 135 | 289 | 30 | 8258 |
| KEGG | AGE-RAGE signaling pathway in | KEGG: 04933 | 2.40E−14 | 13.61917 | 99 | 289 | 26 | 8258 |
| | diabetic complications | | | | | | | |
| KEGG | Endocrine resistance | KEGG: 01522 | 2.88E−14 | 13.54125 | 91 | 289 | 25 | 8258 |
| KEGG | Ras signaling pathway | KEGG: 04014 | 3.59E−14 | 13.44541 | 228 | 289 | 38 | 8258 |
| KEGG | Sphingolipid signaling pathway | KEGG: 04071 | 3.67E−14 | 13.43496 | 119 | 289 | 28 | 8258 |
| KEGG | HIF-1 signaling pathway | KEGG: 04066 | 3.08E−13 | 12.51189 | 109 | 289 | 26 | 8258 |
| KEGG | Amyotrophic lateral sclerosis (ALS) | KEGG: 05014 | 3.27E−13 | 12.4861 | 52 | 289 | 19 | 8258 |
| KEGG | Chagas disease (American | KEGG: 05142 | 4.23E−13 | 12.3741 | 101 | 289 | 25 | 8258 |
| | trypanosomiasis) | | | | | | | |
| KEGG | EGFR tyrosinekinase inhibitor | KEGG: 01521 | 5.60E−13 | 12.25212 | 76 | 289 | 22 | 8258 |
| | resistance | | | | | | | |
| KEGG | Retrograde endocannabinoid signaling | KEGG: 04723 | 7.24E−13 | 12.14021 | 143 | 289 | 29 | 8258 |
| KEGG | Circadian entrainment | KEGG: 04713 | 1.20E−12 | 11.92054 | 96 | 289 | 24 | 8258 |
| KEGG | PI3K-Akt signaling pathway | KEGG: 04151 | 1.31E−12 | 11.88425 | 350 | 289 | 45 | 8258 |
| KEGG | Hepatitis B | KEGG: 05161 | 1.88E−12 | 11.72531 | 159 | 289 | 30 | 8258 |
| KEGG | Human T-cell leukemia virus 1 | KEGG: 05166 | 3.03E−12 | 11.51877 | 234 | 289 | 36 | 8258 |
| | infection | | | | | | | |
| KEGG | Cholinergic synapse | KEGG: 04725 | 3.59E−12 | 11.44494 | 110 | 289 | 25 | 8258 |
| KEGG | Prion diseases | KEGG: 05020 | 1.03E−11 | 10.98551 | 34 | 289 | 15 | 8258 |
| KEGG | cAMP signaling pathway | KEGG: 04024 | 1.83E−11 | 10.73855 | 209 | 289 | 33 | 8258 |
| KEGG | TNF signaling pathway | KEGG: 04668 | 2.52E−11 | 10.59823 | 109 | 289 | 24 | 8258 |
| KEGG | Long-term depression | KEGG: 04730 | 6.53E−11 | 10.18511 | 59 | 289 | 18 | 8258 |
| KEGG | Human immunodeficiency virus 1 | KEGG: 05170 | 1.36E−10 | 9.866462 | 224 | 289 | 33 | 8258 |
| | infection | | | | | | | |
| KEGG | ErbB signaling pathway | KEGG: 04012 | 2.71E−10 | 9.566307 | 81 | 289 | 20 | 8258 |
| KEGG | Cocaine addiction | KEGG: 05030 | 2.77E−10 | 9.558236 | 48 | 289 | 16 | 8258 |
| KEGG | VEGF signaling pathway | KEGG: 04370 | 3.19E−10 | 9.496367 | 56 | 289 | 17 | 8258 |
| KEGG | Chemokine signaling pathway | KEGG: 04062 | 3.27E−10 | 9.486057 | 192 | 289 | 30 | 8258 |
| KEGG | Focal adhesion | KEGG: 04510 | 4.30E−10 | 9.366951 | 194 | 289 | 30 | 8258 |
| KEGG | Gap junction | KEGG: 04540 | 7.15E−10 | 9.145548 | 85 | 289 | 20 | 8258 |
| KEGG | Neurotrophin signaling pathway | KEGG: 04722 | 1.20E−09 | 8.919614 | 118 | 289 | 23 | 8258 |
| KEGG | Serotonergic synapse | KEGG: 04726 | 1.43E−09 | 8.844398 | 130 | 289 | 24 | 8258 |
| KEGG | Osteoclast differentiation | KEGG: 04380 | 2.07E−09 | 8.68355 | 121 | 289 | 23 | 8258 |
| KEGG | Apelin signaling pathway | KEGG: 04371 | 2.80E−09 | 8.552086 | 134 | 289 | 24 | 8258 |
| KEGG | Amoebiasis | KEGG: 05146 | 3.97E−09 | 8.401241 | 103 | 289 | 21 | 8258 |
| KEGG | Parathyroid hormone synthesis, | KEGG: 04928 | 4.82E−09 | 8.317035 | 104 | 289 | 21 | 8258 |
| | secretion and action | | | | | | | |
| KEGG | B cell receptor signaling pathway | KEGG: 04662 | 9.32E−09 | 8.030483 | 77 | 289 | 18 | 8258 |
| KEGG | Alzheimer disease | KEGG: 05010 | 9.35E−09 | 8.029398 | 166 | 289 | 26 | 8258 |
| KEGG | Estrogen signaling pathway | KEGG: 04915 | 1.32E−08 | 7.87939 | 132 | 289 | 23 | 8258 |
| KEGG | T cell receptor signaling pathway | KEGG: 04660 | 1.70E−08 | 7.770066 | 100 | 289 | 20 | 8258 |
| KEGG | Calcium signaling pathway | KEGG: 04020 | 3.90E−08 | 7.408456 | 190 | 289 | 27 | 8258 |
| KEGG | Axon guidance | KEGG: 04360 | 5.16E−08 | 7.287537 | 179 | 289 | 26 | 8258 |
| KEGG | Pertussis | KEGG: 05133 | 5.29E−08 | 7.276605 | 75 | 289 | 17 | 8258 |
| KEGG | GnRH secretion | KEGG: 04929 | 7.76E−08 | 7.110335 | 58 | 289 | 15 | 8258 |
| KEGG | Longevity regulating pathway | KEGG: 04211 | 9.73E−08 | 7.011845 | 88 | 289 | 18 | 8258 |
| KEGG | GABAergic synapse | KEGG: 04727 | 9.73E−08 | 7.011845 | 88 | 289 | 18 | 8258 |
| KEGG | Inflammatory mediator regulation of | KEGG: 04750 | 1.09E−07 | 6.961059 | 122 | 289 | 21 | 8258 |
| | TRP channels | | | | | | | |
| KEGG | Hepatitis C | KEGG: 05160 | 1.11E−07 | 6.954288 | 159 | 289 | 24 | 8258 |
| KEGG | Longevity regulating pathway - | KEGG: 04213 | 1.30E−07 | 6.885467 | 60 | 289 | 15 | 8258 |
| | multiple species | | | | | | | |
| KEGG | FoxO signaling pathway | KEGG: 04068 | 2.02E−07 | 6.695139 | 126 | 289 | 21 | 8258 |
| KEGG | Mitophagy - animal | KEGG: 04137 | 2.72E−07 | 6.565704 | 63 | 289 | 15 | 8258 |
| KEGG | Chronic myeloid leukemia | KEGG: 05220 | 4.46E−07 | 6.350634 | 75 | 289 | 16 | 8258 |
| KEGG | Autophagy - animal | KEGG: 04140 | 4.82E−07 | 6.316554 | 132 | 289 | 21 | 8258 |
| KEGG | Non-alcoholic fatty liver disease | KEGG: 04932 | 5.12E−07 | 6.290895 | 145 | 289 | 22 | 8258 |
| | (NAFLD) | | | | | | | |
| KEGG | C-type lectin receptor signaling | KEGG: 04625 | 5.61E−07 | 6.250661 | 109 | 289 | 19 | 8258 |
| | pathway | | | | | | | |
| KEGG | Nicotine addiction | KEGG: 05033 | 6.27E−07 | 6.202453 | 40 | 289 | 12 | 8258 |
| KEGG | Tuberculosis | KEGG: 05152 | 7.97E−07 | 6.098531 | 175 | 289 | 24 | 8258 |
| KEGG | Acute myeloid leukemia | KEGG: 05221 | 8.43E−07 | 6.074158 | 68 | 289 | 15 | 8258 |
| KEGG | Thl7 cell differentiation | KEGG: 04659 | 9.94E−07 | 6.002416 | 101 | 289 | 18 | 8258 |
| KEGG | Prolactin signaling pathway | KEGG: 04917 | 1.29E−06 | 5.890427 | 70 | 289 | 15 | 8258 |
| KEGG | Melanoma | KEGG: 05218 | 1.58E−06 | 5.801085 | 71 | 289 | 15 | 8258 |
| KEGG | Insulin resistance | KEGG: 04931 | 1.61E−06 | 5.793425 | 104 | 289 | 18 | 8258 |
| KEGG | Measles | KEGG: 05162 | 1.84E−06 | 5.734025 | 142 | 289 | 21 | 8258 |
| KEGG | Adrenergic signaling in | KEGG: 04261 | 2.38E−06 | 5.624117 | 144 | 289 | 21 | 8258 |
| | cardiomyocytes | | | | | | | |
| KEGG | Toxoplasmosis | KEGG: 05145 | 2.56E−06 | 5.592219 | 107 | 289 | 18 | 8258 |
| KEGG | Phospholipase D signaling pathway | KEGG: 04072 | 3.05E−06 | 5.516254 | 146 | 289 | 21 | 8258 |
| KEGG | Wnt signaling pathway | KEGG: 04310 | 3.27E−06 | 5.485011 | 160 | 289 | 22 | 8258 |
| KEGG | Toll-like receptor signaling pathway | KEGG: 04620 | 3.36E−06 | 5.474249 | 97 | 289 | 17 | 8258 |
| KEGG | Aldosterone synthesis and secretion | KEGG: 04925 | 4.6E−06 | 5.337193 | 99 | 289 | 17 | 8258 |
| KEGG | Influenza A | KEGG: 05164 | 5.16E−06 | 5.287334 | 164 | 289 | 22 | 8258 |
| KEGG | Huntington disease | KEGG: 05016 | 9.65E−06 | 5.015584 | 184 | 289 | 23 | 8258 |
| KEGG | Cushing syndrome | KEGG: 04934 | 9.87E−06 | 5.00561 | 156 | 289 | 21 | 8258 |
| KEGG | Platelet activation | KEGG: 04611 | 1.39E−05 | 4.856431 | 119 | 289 | 18 | 8258 |
| KEGG | Yersinia infection | KEGG: 05135 | 1.39E−05 | 4.856431 | 119 | 289 | 18 | 8258 |
| KEGG | Insulin secretion | KEGG: 04911 | 1.44E−05 | 4.841933 | 83 | 289 | 15 | 8258 |
| KEGG | Natural killer cell mediated | KEGG: 04650 | 2.27E−05 | 4.643763 | 110 | 289 | 17 | 8258 |
| | cytotoxicity | | | | | | | |
| KEGG | Fluid shear stress and atherosclerosis | KEGG: 05418 | 2.86E−05 | 4.543652 | 138 | 289 | 19 | 8258 |
| KEGG | Fc epsilon RI signaling pathway | KEGG: 04664 | 2.9E−05 | 4.537334 | 65 | 289 | 13 | 8258 |
| KEGG | Morphine addiction | KEGG: 05032 | 3.2E−05 | 4.494191 | 88 | 289 | 15 | 8258 |
| KEGG | cGMP-PKG signaling pathway | KEGG: 04022 | 3.21E−05 | 4.493502 | 167 | 289 | 21 | 8258 |
| KEGG | Cellular senescence | KEGG: 04218 | 4.35E−05 | 4.361928 | 170 | 289 | 21 | 8258 |
| KEGG | Glucagon signaling pathway | KEGG: 04922 | 4.83E−05 | 4.316014 | 103 | 289 | 16 | 8258 |
| KEGG | Endocrine and other factor-regulated | KEGG: 04961 | 6.92E−05 | 4.159592 | 59 | 289 | 12 | 8258 |
| | calcium reabsorption | | | | | | | |
| KEGG | Apoptosis - multiple species | KEGG: 04215 | 9.64E−05 | 4.016025 | 32 | 289 | 9 | 8258 |
| KEGG | Thyroid hormone synthesis | KEGG: 04918 | 0.000101 | 3.997653 | 72 | 289 | 13 | 8258 |
| KEGG | Th1 and Th2 cell differentiation | KEGG: 04658 | 0.000142 | 3.846598 | 86 | 289 | 14 | 8258 |
| KEGG | Epstein-Barr virus infection | KEGG: 05169 | 0.000189 | 3.72379 | 217 | 289 | 23 | 8258 |
| KEGG | Regulation of lipolysis in adipocytes | KEGG: 04923 | 0.000243 | 3.614982 | 55 | 289 | 11 | 8258 |
| KEGG | mTOR signaling pathway | KEGG: 04150 | 0.000337 | 3.472881 | 147 | 289 | 18 | 8258 |
| KEGG | Phosphatidylinositol signaling system | KEGG: 04070 | 0.000373 | 3.42805 | 93 | 289 | 14 | 8258 |
| KEGG | Type II diabetes mellitus | KEGG: 04930 | 0.000389 | 3.410212 | 47 | 289 | 10 | 8258 |
| KEGG | Rheumatoid arthritis | KEGG: 05323 | 0.000531 | 3.275032 | 83 | 289 | 13 | 8258 |
| KEGG | Progesterone-mediated oocyte | KEGG: 04914 | 0.000795 | 3.099656 | 86 | 289 | 13 | 8258 |
| | maturation | | | | | | | |
| KEGG | Salivary secretion | KEGG: 04970 | 0.001127 | 2.948153 | 76 | 289 | 12 | 8258 |
| KEGG | Vascular smooth muscle contraction | KEGG: 04270 | 0.001982 | 2.702835 | 136 | 289 | 16 | 8258 |
| KEGG | Leishmaniasis | KEGG: 05140 | 0.002073 | 2.683304 | 68 | 289 | 11 | 8258 |
| KEGG | Legionellosis | KEGG: 05134 | 0.002787 | 2.55493 | 58 | 289 | 10 | 8258 |
| KEGG | Adipocytokine signaling pathway | KEGG: 04920 | 0.003154 | 2.501076 | 71 | 289 | 11 | 8258 |
| KEGG | Viral protein interaction with cytokine | KEGG: 04061 | 0.004186 | 2.378164 | 100 | 289 | 13 | 8258 |
| | and cytokine receptor | | | | | | | |
| KEGG | Platinum drug resistance | KEGG: 01524 | 0.004691 | 2.328775 | 74 | 289 | 11 | 8258 |
| KEGG | Synaptic vesicle cycle | KEGG: 04721 | 0.004691 | 2.328775 | 74 | 289 | 11 | 8258 |
| KEGG | IL-17 signaling pathway | KEGG: 04657 | 0.005127 | 2.290156 | 88 | 289 | 12 | 8258 |
| KEGG | JAK-STAT signaling pathway | KEGG: 04630 | 0.005278 | 2.277534 | 163 | 289 | 17 | 8258 |
| KEGG | Insulin signaling pathway | KEGG: 04910 | 0.005932 | 2.226821 | 133 | 289 | 15 | 8258 |
| KEGG | Leukocyte transendothelial migration | KEGG: 04670 | 0.012529 | 1.902077 | 111 | 289 | 13 | 8258 |
| KEGG | Cortisol synthesis and secretion | KEGG: 04927 | 0.012924 | 1.888587 | 69 | 289 | 10 | 8258 |
| KEGG | African trypanosomiasis | KEGG: 05143 | 0.017367 | 1.760274 | 35 | 289 | 7 | 8258 |
| KEGG | NOD-like receptor signaling pathway | KEGG: 04621 | 0.017872 | 1.747834 | 197 | 289 | 18 | 8258 |
| KEGG | Alcoholism | KEGG: 05034 | 0.017872 | 1.747834 | 197 | 289 | 18 | 8258 |
| KEGG | Fc gamma R-mediated phagocytosis | KEGG: 04666 | 0.018912 | 1.723255 | 86 | 289 | 11 | 8258 |
| KEGG | Salmonella infection | KEGG: 05132 | 0.026184 | 1.581972 | 75 | 289 | 10 | 8258 |
| KEGG | Lysosome | KEGG: 04142 | 0.032486 | 1.488303 | 122 | 289 | 13 | 8258 |
| KEGG | Necroptosis | KEGG: 04217 | 0.035536 | 1.449331 | 173 | 289 | 16 | 8258 |
| KEGG | Hematopoietic cell lineage | KEGG: 04640 | 0.041653 | 1.380352 | 94 | 289 | 11 | 8258 |

TABLE 13
From TTPB_0400_final document, Differentially Expressed Genes - GProfiler Results - DIV03

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Glutamatergic synapse | KEGG: 04724 | 3.93E−28 | 27.40555 | 112 | 338 | 42 | 8258 |
| KEGG | Dopaminergic synapse | KEGG: 04728 | 2.38E−23 | 22.62274 | 127 | 338 | 40 | 8258 |
| KEGG | MAPK signaling pathway | KEGG: 04010 | 4.29E−20 | 19.36787 | 286 | 338 | 54 | 8258 |
| KEGG | Long-term potentiation | KEGG: 04720 | 9.63E−20 | 19.01643 | 62 | 338 | 27 | 8258 |
| KEGG | Retrograde endocannabinoid signaling | KEGG: 04723 | 2.99E−17 | 16.52393 | 143 | 338 | 36 | 8258 |
| KEGG | Human cytomegalovirus infection | KEGG: 05163 | 5.24E−17 | 16.28101 | 242 | 338 | 46 | 8258 |
| KEGG | Cholinergic synapse | KEGG: 04725 | 2.95E−16 | 15.53012 | 110 | 338 | 31 | 8258 |
| KEGG | Circadian entrainment | KEGG: 04713 | 4.62E−16 | 15.33565 | 96 | 338 | 29 | 8258 |
| KEGG | Amphetamine addiction | KEGG: 05031 | 1.29E−15 | 14.88777 | 64 | 338 | 24 | 8258 |
| KEGG | cAMP signaling pathway | KEGG: 04024 | 1.56E−15 | 14.80707 | 209 | 338 | 41 | 8258 |
| KEGG | HIF-1 signaling pathway | KEGG: 04066 | 2.21E−15 | 14.65596 | 109 | 338 | 30 | 8258 |
| KEGG | Long-term depression | KEGG: 04730 | 2.26E−15 | 14.6463 | 59 | 338 | 23 | 8258 |
| KEGG | Oxytocin signaling pathway | KEGG: 04921 | 5.37E−15 | 14.27031 | 147 | 338 | 34 | 8258 |
| KEGG | Relaxin signaling pathway | KEGG: 04926 | 2.20E−13 | 12.65839 | 127 | 338 | 30 | 8258 |
| KEGG | Amyotrophic lateral sclerosis (ALS) | KEGG: 05014 | 4.01E−13 | 12.39717 | 52 | 338 | 20 | 8258 |
| KEGG | Calcium signaling pathway | KEGG: 04020 | 5.29E−13 | 12.27672 | 190 | 338 | 36 | 8258 |
| KEGG | Endocrine resistance | KEGG: 01522 | 1.20E−12 | 11.92193 | 91 | 338 | 25 | 8258 |
| KEGG | EGFR tyrosine kinase inhibitor | KEGG: 01521 | 1.41E−12 | 11.84958 | 76 | 338 | 23 | 8258 |
| | resistance | | | | | | | |
| KEGG | Rap1 signaling pathway | KEGG: 04015 | 2.07E−12 | 11.68496 | 209 | 338 | 37 | 8258 |
| KEGG | Sphingolipid signaling pathway | KEGG: 04071 | 2.19E−12 | 11.6586 | 119 | 338 | 28 | 8258 |
| KEGG | GnRH signaling pathway | KEGG: 04912 | 4.96E−12 | 11.30443 | 88 | 338 | 24 | 8258 |
| KEGG | PI3K-Akt signaling pathway | KEGG: 04151 | 6.28E−12 | 11.20185 | 350 | 338 | 48 | 8258 |
| KEGG | Nicotine addiction | KEGG: 05033 | 6.75E−12 | 11.17071 | 40 | 338 | 17 | 8258 |
| KEGG | Apelin signaling pathway | KEGG: 04371 | 7.61E−12 | 11.1185 | 134 | 338 | 29 | 8258 |
| KEGG | AGE-RAGE signaling pathway in | KEGG: 04933 | 1.01E−11 | 10.99646 | 99 | 338 | 25 | 8258 |
| | diabetic complications | | | | | | | |
| KEGG | Pertussis | KEGG: 05133 | 1.11E−11 | 10.95349 | 75 | 338 | 22 | 8258 |
| KEGG | Human T-cell leukemia virus 1 | KEGG: 05166 | 1.58E−11 | 10.80178 | 234 | 338 | 38 | 8258 |
| | infection | | | | | | | |
| KEGG | Chagas disease (American | KEGG: 05142 | 1.66E−11 | 10.7805 | 101 | 338 | 25 | 8258 |
| | trypanosomiasis) | | | | | | | |
| KEGG | Serotonergic synapse | KEGG: 04726 | 2.38E−11 | 10.62419 | 130 | 338 | 28 | 8258 |
| KEGG | VEGF signaling pathway | KEGG: 04370 | 2.89E−11 | 10.53901 | 56 | 338 | 19 | 8258 |
| KEGG | Ras signaling pathway | KEGG: 04014 | 3.46E−11 | 10.46114 | 228 | 338 | 37 | 8258 |
| KEGG | ErbB signaling pathway | KEGG: 04012 | 6.40E−11 | 10.19363 | 81 | 338 | 22 | 8258 |
| KEGG | Apoptosis | KEGG: 04210 | 6.43E−11 | 10.19181 | 135 | 338 | 28 | 8258 |
| KEGG | Focal adhesion | KEGG: 04510 | 1.86E−10 | 9.730179 | 194 | 338 | 33 | 8258 |
| KEGG | GABAergic synapse | KEGG: 04727 | 4.01E−10 | 9.396315 | 88 | 338 | 22 | 8258 |
| KEGG | Human immunodeficiency virus 1 | KEGG: 05170 | 4.89E−10 | 9.310272 | 224 | 338 | 35 | 8258 |
| | infection | | | | | | | |
| KEGG | Hepatitis B | KEGG: 05161 | 7.41E−10 | 9.130098 | 159 | 338 | 29 | 8258 |
| KEGG | TNF signaling pathway | KEGG: 04668 | 7.93E−10 | 9.100983 | 109 | 338 | 24 | 8258 |
| KEGG | Cocaine addiction | KEGG: 05030 | 3.14E−09 | 8.503578 | 48 | 338 | 16 | 8258 |
| KEGG | Thyroid hormone signaling pathway | KEGG: 04919 | 3.25E−09 | 8.48873 | 116 | 338 | 24 | 8258 |
| KEGG | FoxO signaling pathway | KEGG: 04068 | 3.27E−09 | 8.484816 | 126 | 338 | 25 | 8258 |
| KEGG | Osteoclast differentiation | KEGG: 04380 | 8.30E−09 | 8.08087 | 121 | 338 | 24 | 8258 |
| KEGG | Tuberculosis | KEGG: 05152 | 8.57E−09 | 8.067181 | 175 | 338 | 29 | 8258 |
| KEGG | Toxoplasmosis | KEGG: 05145 | 2.52E−08 | 7.59921 | 107 | 338 | 22 | 8258 |
| KEGG | Neurotrophin signaling pathway | KEGG: 04722 | 3.01E−08 | 7.520845 | 118 | 338 | 23 | 8258 |
| KEGG | Prion diseases | KEGG: 05020 | 3.22E−08 | 7.491706 | 34 | 338 | 13 | 8258 |
| KEGG | Alzheimer disease | KEGG: 05010 | 6.11E−08 | 7.214155 | 166 | 338 | 27 | 8258 |
| KEGG | Morphine addiction | KEGG: 05032 | 1.83E−07 | 6.738226 | 88 | 338 | 19 | 8258 |
| KEGG | Platelet activation | KEGG: 04611 | 2.14E−07 | 6.670446 | 119 | 338 | 22 | 8258 |
| KEGG | C-type lectin receptor signaling | KEGG: 04625 | 2.29E−07 | 6.639482 | 109 | 338 | 21 | 8258 |
| | pathway | | | | | | | |
| KEGG | T cell receptor signaling pathway | KEGG: 04660 | 2.82E−07 | 6.550032 | 100 | 338 | 20 | 8258 |
| KEGG | Estrogen signaling pathway | KEGG: 04915 | 3.05E−07 | 6.516363 | 132 | 338 | 23 | 8258 |
| KEGG | cGMP-PKG signaling pathway | KEGG: 04022 | 3.39E−07 | 6.46979 | 167 | 338 | 26 | 8258 |
| KEGG | Chemokine signaling pathway | KEGG: 04062 | 3.76E−07 | 6.4246 | 192 | 338 | 28 | 8258 |
| KEGG | Parathyroid hormone synthesis, | KEGG: 04928 | 5.78E−07 | 6.238044 | 104 | 338 | 20 | 8258 |
| | secretion and action | | | | | | | |
| KEGG | Insulin resistance | KEGG: 04931 | 5.78E−07 | 6.238044 | 104 | 338 | 20 | 8258 |
| KEGG | Human papillomavirus infection | KEGG: 05165 | 6.36E−07 | 6.196243 | 346 | 338 | 39 | 8258 |
| KEGG | GnRH secretion | KEGG: 04929 | 7.01E−07 | 6.154113 | 58 | 338 | 15 | 8258 |
| KEGG | Longevity regulating pathway - | KEGG: 04213 | 1.16E−06 | 5.934211 | 60 | 338 | 15 | 8258 |
| | multiple species | | | | | | | |
| KEGG | Longevity regulating pathway | KEGG: 04211 | 1.23E−06 | 5.909862 | 88 | 338 | 18 | 8258 |
| KEGG | Inflammatory mediator regulation of | KEGG: 04750 | 1.9E−06 | 5.722252 | 122 | 338 | 21 | 8258 |
| | TRP channels | | | | | | | |
| KEGG | Amoebiasis | KEGG: 05146 | 2.87E−06 | 5.542063 | 103 | 338 | 19 | 8258 |
| KEGG | Synaptic vesicle cycle | KEGG: 04721 | 3.54E−06 | 5.451346 | 74 | 338 | 16 | 8258 |
| KEGG | Gap junction | KEGG: 04540 | 4.5E−06 | 5.346485 | 85 | 338 | 17 | 8258 |
| KEGG | Autophagy - animal | KEGG: 04140 | 7.89E−06 | 5.103068 | 132 | 338 | 21 | 8258 |
| KEGG | Thl7 cell differentiation | KEGG: 04659 | 1.17E−05 | 4.933031 | 101 | 338 | 18 | 8258 |
| KEGG | Glucagon signaling pathway | KEGG: 04922 | 1.59E−05 | 4.797821 | 103 | 338 | 18 | 8258 |
| KEGG | Fluid shear stress and atherosclerosis | KEGG: 05418 | 1.73E−05 | 4.761921 | 138 | 338 | 21 | 8258 |
| KEGG | Axon guidance | KEGG: 04360 | 2.61E−05 | 4.582925 | 179 | 338 | 24 | 8258 |
| KEGG | Fc epsilon RI signaling pathway | KEGG: 04664 | 2.72E−05 | 4.564743 | 65 | 338 | 14 | 8258 |
| KEGG | Type II diabetes mellitus | KEGG: 04930 | 2.74E−05 | 4.56219 | 47 | 338 | 12 | 8258 |
| KEGG | Measles | KEGG: 05162 | 2.85E−05 | 4.545824 | 142 | 338 | 21 | 8258 |
| KEGG | Melanogenesis | KEGG: 04916 | 2.91E−05 | 4.535845 | 96 | 338 | 17 | 8258 |
| KEGG | Yersinia infection | KEGG: 05135 | 3.15E−05 | 4.501662 | 119 | 338 | 19 | 8258 |
| KEGG | Apoptosis - multiple species | KEGG: 04215 | 3.5E−05 | 4.455585 | 32 | 338 | 10 | 8258 |
| KEGG | Huntington disease | KEGG: 05016 | 4.39E−05 | 4.357306 | 184 | 338 | 24 | 8258 |
| KEGG | Aldosterone synthesis and secretion | KEGG: 04925 | 4.61E−05 | 4.336334 | 99 | 338 | 17 | 8258 |
| KEGG | Acute myeloid leukemia | KEGG: 05221 | 4.92E−05 | 4.307775 | 68 | 338 | 14 | 8258 |
| KEGG | Platinum drug resistance | KEGG: 01524 | 0.000146 | 3.836464 | 74 | 338 | 14 | 8258 |
| KEGG | Cellular senescence | KEGG: 04218 | 0.000154 | 3.811224 | 170 | 338 | 22 | 8258 |
| KEGG | Toll-like receptor signaling pathway | KEGG: 04620 | 0.000177 | 3.752948 | 97 | 338 | 16 | 8258 |
| KEGG | Fc gamma R-mediated phagocytosis | KEGG: 04666 | 0.000181 | 3.743142 | 86 | 338 | 15 | 8258 |
| KEGG | Phospholipase D signaling pathway | KEGG: 04072 | 0.000191 | 3.719478 | 146 | 338 | 20 | 8258 |
| KEGG | B cell receptor signaling pathway | KEGG: 04662 | 0.00024 | 3.619598 | 77 | 338 | 14 | 8258 |
| KEGG | Insulin secretion | KEGG: 04911 | 0.000605 | 3.218303 | 83 | 338 | 14 | 8258 |
| KEGG | Hepatitis C | KEGG: 05160 | 0.000725 | 3.139588 | 159 | 338 | 20 | 8258 |
| KEGG | Mitophagy - animal | KEGG: 04137 | 0.000775 | 3.110793 | 63 | 338 | 12 | 8258 |
| KEGG | Wnt signaling pathway | KEGG: 04310 | 0.000798 | 3.097867 | 160 | 338 | 20 | 8258 |
| KEGG | Influenza A | KEGG: 05164 | 0.001162 | 2.934781 | 164 | 338 | 20 | 8258 |
| KEGG | Neuroactive ligand-receptor interaction | KEGG: 04080 | 0.001398 | 2.854369 | 348 | 338 | 32 | 8258 |
| KEGG | Cushing syndrome | KEGG: 04934 | 0.001966 | 2.706326 | 156 | 338 | 19 | 8258 |
| KEGG | Rheumatoid arthritis | KEGG: 05323 | 0.002982 | 2.525435 | 83 | 338 | 13 | 8258 |
| KEGG | Th1 and Th2 cell differentiation | KEGG: 04658 | 0.004392 | 2.357312 | 86 | 338 | 13 | 8258 |
| KEGG | Viral protein interaction with cytokine | KEGG: 04061 | 0.005379 | 2.26932 | 100 | 338 | 14 | 8258 |
| | and cytokine receptor | | | | | | | |
| KEGG | Alcoholism | KEGG: 05034 | 0.005497 | 2.259872 | 197 | 338 | 21 | 8258 |
| KEGG | Epstein-Barr virus infection | KEGG: 05169 | 0.007832 | 2.10614 | 217 | 338 | 22 | 8258 |
| KEGG | Leishmaniasis | KEGG: 05140 | 0.009101 | 2.040919 | 68 | 338 | 11 | 8258 |
| KEGG | Insulin signaling pathway | KEGG: 04910 | 0.010424 | 1.981948 | 133 | 338 | 16 | 8258 |
| KEGG | mTOR signaling pathway | KEGG: 04150 | 0.010445 | 1.981074 | 147 | 338 | 17 | 8258 |
| KEGG | Legionellosis | KEGG: 05134 | 0.010963 | 1.960059 | 58 | 338 | 10 | 8258 |
| KEGG | JAK-STAT signaling pathway | KEGG: 04630 | 0.011971 | 1.921887 | 163 | 338 | 18 | 8258 |
| KEGG | Endocrine and other factor-regulated | KEGG: 04961 | 0.012729 | 1.8952 | 59 | 338 | 10 | 8258 |
| | calcium reabsorption | | | | | | | |
| KEGG | AMPK signaling pathway | KEGG: 04152 | 0.013386 | 1.87334 | 122 | 338 | 15 | 8258 |
| KEGG | Natural killer cell mediated | KEGG: 04650 | 0.015442 | 1.811291 | 110 | 338 | 14 | 8258 |
| | cytotoxicity | | | | | | | |
| KEGG | Leukocyte transendothelial migration | KEGG: 04670 | 0.017029 | 1.768807 | 111 | 338 | 14 | 8258 |
| KEGG | Salmonella infection | KEGG: 05132 | 0.022511 | 1.647614 | 75 | 338 | 11 | 8258 |
| KEGG | Hematopoietic cell lineage | KEGG: 04640 | 0.044337 | 1.353235 | 94 | 338 | 12 | 8258 |
| KEGG | Vascular smooth muscle contraction | KEGG: 04270 | 0.044729 | 1.349406 | 136 | 338 | 15 | 8258 |
| KEGG | Tight junction | KEGG: 04530 | 0.045567 | 1.34135 | 166 | 338 | 17 | 8258 |

TABLE 14
From TTPB_0400_final document, Differentially Expressed Genes - GProfiler Results - DIV04

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Glutamatergic synapse | KEGG: 047 | 2.93E−26 | 25.53266 | 112 | 306 | 39 | 8258 |
| KEGG | Long-term potentiation | KEGG: 047 | 3.01E−22 | 21.52177 | 62 | 306 | 28 | 8258 |
| KEGG | Dopaminergic synapse | KEGG: 047 | 1.43E−19 | 18.84328 | 127 | 306 | 35 | 8258 |
| KEGG | MAPK signaling pathway | KEGG: 040 | 6.77E−19 | 18.1697 | 286 | 306 | 50 | 8258 |
| KEGG | Circadian entrainment | KEGG: 047 | 2.34E−18 | 17.63096 | 96 | 306 | 30 | 8258 |
| KEGG | Oxytocin signaling pathway | KEGG: 049 | 2.87E−18 | 17.5416 | 147 | 306 | 36 | 8258 |
| KEGG | Amphetamine addiction | KEGG: 050 | 7.37E−18 | 17.13279 | 64 | 306 | 25 | 8258 |
| KEGG | Retrograde endocannabinoid signaling | KEGG: 047 | 1.02E−17 | 16.99198 | 143 | 306 | 35 | 8258 |
| KEGG | Cholinergic synapse | KEGG: 047 | 1.61E−17 | 16.7926 | 110 | 306 | 31 | 8258 |
| KEGG | Human cytomegalovirus infection | KEGG: 051 | 3.98E−17 | 16.40048 | 242 | 306 | 44 | 8258 |
| KEGG | cAMP signaling pathway | KEGG: C40 | 1.96E−15 | 14.70823 | 209 | 306 | 39 | 8258 |
| KEGG | GnRH signaling pathway | KEGG: 049 | 4.26E−15 | 14.37074 | 88 | 306 | 26 | 8258 |
| KEGG | HIF-1 signaling pathway | KEGG: C40 | 1.47E−14 | 13.83292 | 109 | 306 | 28 | 8258 |
| KEGG | GABAergic synapse | KEGG: 047 | 4.96E−14 | 13.30449 | 88 | 306 | 25 | 8258 |
| KEGG | Long-term depression | KEGG: 047 | 6.97E−14 | 13.15706 | 59 | 306 | 21 | 8258 |
| KEGG | Calcium signaling pathway | KEGG: 040 | 1.5CE−13 | 12.82288 | 190 | 306 | 35 | 8258 |
| KEGG | EGFR tyrosine kinase inhibitor | KEGG: 015 | 1.66E−13 | 12.78076 | 76 | 306 | 23 | 8258 |
| | resistance | | | | | | | |
| KEGG | Serotonergic synapse | KEGG: 047 | 2.44E−13 | 12.61339 | 130 | 306 | 29 | 8258 |
| KEGG | Rap1 signaling pathway | KEGG: 040 | 5.2CE−13 | 12.28359 | 209 | 306 | 36 | 8258 |
| KEGG | Endocrine resistance | KEGG: 015 | 1.25E−12 | 11.90308 | 91 | 306 | 24 | 8258 |
| KEGG | PI3K-Akt signaling pathway | KEGG: 041 | 2.66E−12 | 11.57538 | 350 | 306 | 46 | 8258 |
| KEGG | Relaxin signaling pathway | KEGG: 049 | 8.29E−12 | 11.08168 | 127 | 306 | 27 | 8258 |
| KEGG | Nicotine addiction | KEGG: 060 | 2.59E−11 | 10.58748 | 40 | 306 | 16 | 8258 |
| KEGG | Ras signaling pathway | KEGG: 040 | 4.55E−11 | 10.34193 | 228 | 306 | 35 | 8258 |
| KEGG | AGE-RAGE signaling pathway in | KEGG: 049 | 8.58E−11 | 10.06631 | 99 | 306 | 23 | 8258 |
| | diabetic complications | | | | | | | |
| KEGG | Sphingolipid signaling pathway | KEGG: 040 | 9.61E−11 | 10.01714 | 119 | 306 | 25 | 8258 |
| KEGG | Human T-cell leukemia virus 1 | KEGG: 061 | 9.97E−11 | 10.00111 | 234 | 306 | 35 | 8258 |
| | infection | | | | | | | |
| KEGG | Amyotrophic lateral sclerosis (ALS) | KEGG: 060 | 2.19E−10 | 9.659108 | 52 | 306 | 17 | 8258 |
| KEGG | Apelin signaling pathway | KEGG: 043 | 2.33E−10 | 9.632777 | 134 | 306 | 26 | 8258 |
| KEGG | Apoptosis | KEGG: 042 | 2.79E−10 | 9.554434 | 135 | 306 | 26 | 8258 |
| KEGG | Focal adhesion | KEGG: 045 | 3.69E−10 | 9.433019 | 194 | 306 | 31 | 8258 |
| KEGG | ErbB signaling pathway | KEGG: 040 | 8.35E−10 | 9.078108 | 81 | 306 | 20 | 8258 |
| KEGG | Chagas disease (American | KEGG: 061 | 1.10E−09 | 8.959363 | 101 | 306 | 22 | 8258 |
| | trypanosomiasis) | | | | | | | |
| KEGG | Alzheimer disease | KEGG: 060 | 1.14E−09 | 8.942114 | 166 | 306 | 28 | 8258 |
| KEGG | Pertussis | KEGG: 061 | 1.73E−09 | 8.761422 | 75 | 306 | 19 | 8258 |
| KEGG | Parathyroid hormone synthesis, secretion | KEGG: 049 | 2.04E−09 | 8.690133 | 104 | 306 | 22 | 8258 |
| | and action | | | | | | | |
| KEGG | Thyroid hormone signaling pathway | KEGG: 049 | 2.87E−09 | 8.541825 | 116 | 306 | 23 | 8258 |
| KEGG | Toxoplasmosis | KEGG: 061 | 3.71E−09 | 8.430888 | 107 | 306 | 22 | 8258 |
| KEGG | TNF signaling pathway | KEGG: 046 | 5.45E−09 | 8.263295 | 109 | 306 | 22 | 8258 |
| KEGG | Inflammatory mediator regulation of | KEGG: 047 | 8.47E−09 | 8.072311 | 122 | 306 | 23 | 8258 |
| | TRP channels | | | | | | | |
| KEGG | Cocaine addiction | KEGG: 060 | 9.42E−09 | 8.026051 | 48 | 306 | 15 | 8258 |
| KEGG | Prion diseases | KEGG: 060 | 9.53E−09 | 8.020827 | 34 | 306 | 13 | 8258 |
| KEGG | Amoebiasis | KEGG: 061 | 1.24E−08 | 7.905081 | 103 | 306 | 21 | 8258 |
| KEGG | Hepatitis B | KEGG: 061 | 1.33E−08 | 7.877567 | 159 | 306 | 26 | 8258 |
| KEGG | Gap junction | KEGG: 045 | 1.83E−08 | 7.73657 | 85 | 306 | 19 | 8258 |
| KEGG | Estrogen signaling pathway | KEGG: 049 | 4.43E−08 | 7.353997 | 132 | 306 | 23 | 8258 |
| KEGG | Osteoclast differentiation | KEGG: 043 | 4.61E−08 | 7.336305 | 121 | 306 | 22 | 8258 |
| KEGG | Human immunodeficiency virus 1 | KEGG: 061 | 7.58E−08 | 7.12015 | 224 | 306 | 30 | 8258 |
| | infection | | | | | | | |
| KEGG | FoxO signaling pathway | KEGG: 040 | 1.03E−07 | 6.985266 | 126 | 306 | 22 | 8258 |
| KEGG | Human papillomavirus infection | KEGG: 061 | 1.29E−07 | 6.888506 | 346 | 306 | 38 | 8258 |
| KEGG | Melanogenesis | KEGG: 049 | 1.67E−07 | 6.777025 | 96 | 306 | 19 | 8258 |
| KEGG | Neurotrophin signaling pathway | KEGG: 047 | 1.75E−07 | 6.755762 | 118 | 306 | 21 | 8258 |
| KEGG | Chemokine signaling pathway | KEGG: 040 | 1.90E−07 | 6.721141 | 192 | 306 | 27 | 8258 |
| KEGG | Aldosterone synthesis and secretion | KEGG: 049 | 2.88E−07 | 6.540109 | 99 | 306 | 19 | 8258 |
| KEGG | Tuberculosis | KEGG: 061 | 5.62E−07 | 6.250225 | 175 | 306 | 25 | 8258 |
| KEGG | Insulin secretion | KEGG: 049 | 7.17E−07 | 6.144703 | 83 | 306 | 17 | 8258 |
| KEGG | Type II diabetes mellitus | KEGG: 049 | 9.35E−07 | 6.029212 | 47 | 306 | 13 | 8258 |
| KEGG | VEGF signaling pathway | KEGG: 043 | 1.05E−06 | 5.980662 | 56 | 306 | 14 | 8258 |
| KEGG | Platelet activation | KEGG: 046 | 1.21E−06 | 5.915695 | 119 | 306 | 20 | 8258 |
| KEGG | Cushing syndrome | KEGG: 049 | 1.28E−06 | 5.893013 | 156 | 306 | 23 | 8258 |
| KEGG | Longevity regulating pathway | KEGG: 042 | 1.82E−06 | 5.739402 | 88 | 306 | 17 | 8258 |
| KEGG | Morphine addiction | KEGG: 060 | 1.82E−06 | 5.739402 | 88 | 306 | 17 | 8258 |
| KEGG | T cell receptor signaling pathway | KEGG: 046 | 2.21E−06 | 5.656555 | 100 | 306 | 18 | 8258 |
| KEGG | Th17 cell differentiation | KEGG: 046 | 2.59E−06 | 5.585942 | 101 | 306 | 18 | 8258 |
| KEGG | Insulin resistance | KEGG: 049 | 4.17E−06 | 5.379555 | 104 | 306 | 18 | 8258 |
| KEGG | cGMP-PKG signaling pathway | KEGG: 040 | 4.76E−06 | 5.322141 | 167 | 306 | 23 | 8258 |
| KEGG | Autophagy - animal | KEGG: 041 | 7.43E−06 | 5.129117 | 132 | 306 | 20 | 8258 |
| KEGG | Phospholipase D signaling pathway | KEGG: 040 | 8.75E−06 | 5.05786 | 146 | 306 | 21 | 8258 |
| KEGG | GnRH secretion | KEGG: 049 | 1.45E−05 | 4.83816 | 58 | 306 | 13 | 8258 |
| KEGG | Fluid shear stress and atherosclerosis | KEGG: 054 | 1.58E−05 | 4.800725 | 138 | 306 | 20 | 8258 |
| KEGG | Glucagon signaling pathway | KEGG: 049 | 2.06E−05 | 4.685849 | 103 | 306 | 17 | 8258 |
| KEGG | Longevity regulating pathway - multiple | KEGG: 042 | 2.22E−05 | 4.653049 | 60 | 306 | 13 | 8258 |
| | species | | | | | | | |
| KEGG | Yersinia infection | KEGG: 061 | 3.5E−05 | 4.455564 | 119 | 306 | 18 | 8258 |
| KEGG | Wnt signaling pathway | KEGG: 043 | 4.29E−05 | 4.367614 | 160 | 306 | 21 | 8258 |
| KEGG | Vascular smooth muscle contraction | KEGG: 042 | 5.86E−05 | 4.232272 | 136 | 306 | 19 | 8258 |
| KEGG | Axon guidance | KEGG: 043 | 7.16E−05 | 4.144943 | 179 | 306 | 22 | 8258 |
| KEGG | Dilated cardiomyopathy (DCM) | KEGG: 054 | 0.000111 | 3.952967 | 91 | 306 | 15 | 8258 |
| KEGG | Measles | KEGG: 061 | 0.000115 | 3.93847 | 142 | 306 | 19 | 8258 |
| KEGG | Apoptosis - multiple species | KEGG: 042 | 0.000166 | 3.779708 | 32 | 306 | 9 | 8258 |
| KEGG | Adipocytokine signaling pathway | KEGG: 049 | 0.000173 | 3.761654 | 71 | 306 | 13 | 8258 |
| KEGG | Neuroactive ligand-receptor interaction | KEGG: 040 | 0.000173 | 3.761062 | 348 | 306 | 32 | 8258 |
| KEGG | C-type lectin receptor signaling pathway | KEGG: 046 | 0.000241 | 3.618061 | 109 | 306 | 16 | 8258 |
| KEGG | Renin secretion | KEGG: 049 | 0.000282 | 3.54959 | 74 | 306 | 13 | 8258 |
| KEGG | Fc gamma R-mediated phagocytosis | KEGG: 046 | 0.000299 | 3.524042 | 86 | 306 | 14 | 8258 |
| KEGG | Prolactin signaling pathway | KEGG: 049 | 0.000895 | 3.048031 | 70 | 306 | 12 | 8258 |
| KEGG | JAK-STAT signaling pathway | KEGG: 046 | 0.000924 | 3.034148 | 163 | 306 | 19 | 8258 |
| KEGG | Endocrine and other factor-regulated | KEGG: 049 | 0.000925 | 3.033814 | 59 | 306 | 11 | 8258 |
| | calcium reabsorption | | | | | | | |
| KEGG | Toll-like receptor signaling pathway | KEGG: 046 | 0.001276 | 2.89406 | 97 | 306 | 14 | 8258 |
| KEGG | Platinum drug resistance | KEGG: 015 | 0.001617 | 2.791318 | 74 | 306 | 12 | 8258 |
| KEGG | Mitophagy - animal | KEGG: 041 | 0.001783 | 2.7488 | 63 | 306 | 11 | 8258 |
| KEGG | Salivary secretion | KEGG: 049 | 0.002139 | 2.669804 | 76 | 306 | 12 | 8258 |
| KEGG | Hepatitis C | KEGG: 061 | 0.002393 | 2.621069 | 159 | 306 | 18 | 8258 |
| KEGG | Fc epsilon RI signaling pathway | KEGG: 046 | 0.002426 | 2.615089 | 65 | 306 | 11 | 8258 |
| KEGG | B cell receptor signaling pathway | KEGG: 046 | 0.002451 | 2.610643 | 77 | 306 | 12 | 8258 |
| KEGG | Insulin signaling pathway | KEGG: 049 | 0.003232 | 2.490514 | 133 | 306 | 16 | 8258 |
| KEGG | Influenza A | KEGG: C61 | 0.003645 | 2.438296 | 164 | 306 | 18 | 8258 |
| KEGG | Leishmaniasis | KEGG: C61 | 0.003763 | 2.424434 | 68 | 306 | 11 | 8258 |
| KEGG | Cortisol synthesis and secretion | KEGG: 049 | 0.004332 | 2.363356 | 69 | 306 | 11 | 8258 |
| KEGG | Huntington disease | KEGG: C60 | 0.005187 | 2.285123 | 184 | 306 | 19 | 8258 |
| KEGG | Rheumatoid arthritis | KEGG: C63 | 0.005294 | 2.276235 | 83 | 306 | 12 | 8258 |
| KEGG | Thyroid hormone synthesis | KEGG: 049 | 0.006501 | 2.187012 | 72 | 306 | 11 | 8258 |
| KEGG | Th1 and Th2 cell differentiation | KEGG: 046 | 0.007566 | 2.121115 | 86 | 306 | 12 | 8258 |
| KEGG | Viral protein interaction with cytokine | KEGG: 040 | 0.00806 | 2.093661 | 100 | 306 | 13 | 8258 |
| | and cytokine receptor | | | | | | | |
| KEGG | Synaptic vesicle cycle | KEGG: 047 | 0.008417 | 2.074849 | 74 | 306 | 11 | 8258 |
| KEGG | Salmonella infection | KEGG: C61 | 0.009544 | 2.020292 | 75 | 306 | 11 | 8258 |
| KEGG | mTOR signaling pathway | KEGG: 041 | 0.011003 | 1.958478 | 147 | 306 | 16 | 8258 |
| KEGG | Alcoholism | KEGG: C60 | 0.013085 | 1.883213 | 197 | 306 | 19 | 8258 |
| KEGG | NOD-like receptor signaling pathway | KEGG: 046 | 0.013085 | 1.883213 | 197 | 306 | 19 | 8258 |
| KEGG | Hematopoietic cell lineage | KEGG: M& | 0.018131 | 1.741573 | 94 | 306 | 12 | 8258 |
| KEGG | Cellular senescence | KEGG: 042 | 0.019206 | 1.716559 | 170 | 306 | 17 | 8258 |
| KEGG | Cytokine-cytokine receptor interaction | KEGG: 040 | 0.019487 | 1.710251 | 290 | 306 | 24 | 8258 |
| KEGG | Legionellosis | KEGG: C61 | 0.026995 | 1.56872 | 58 | 306 | 9 | 8258 |
| KEGG | Sphingolipid metabolism | KEGG: C06 | 0.030987 | 1.508821 | 47 | 306 | 8 | 8258 |
| KEGG | Aldosterone-regulated sodium | KEGG: 049 | 0.031761 | 1.498103 | 36 | 306 | 7 | 8258 |
| | reabsorption | | | | | | | |
| KEGG | IL-17 signaling pathway | KEGG: 046 | 0.040642 | 1.391026 | 88 | 306 | 11 | 8258 |
| KEGG | Epstein-Barr virus infection | KEGG: C61 | 0.04568 | 1.340278 | 217 | 306 | 19 | 8258 |

TABLE 15
From TTPB_0400_final document, Differentially Expressed Genes - GProfiler Results - DIV06

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Glutamatergic synapse | KEGG: 04724 | 9.45E−30 | 29.02451446 | 112 | 353 | 44 | 8258 |
| KEGG | Dopaminergic synapse | KEGG: 04728 | 1.43E−21 | 20.8432963 | 127 | 353 | 39 | 8258 |
| KEGG | Long-term potentiation | KEGG: 04720 | 1.55E−20 | 19.80858726 | 62 | 353 | 28 | 8258 |
| KEGG | Oxytocin signaling pathway | KEGG: 04921 | 5.79E−20 | 19.23704523 | 147 | 353 | 40 | 8258 |
| KEGG | Circadian entrainment | KEGG: 04713 | 7.88E−20 | 19.10346841 | 96 | 353 | 33 | 8258 |
| KEGG | Retrograde endocannabinoid | KEGG: 04723 | 1.79E−19 | 18.74693954 | 143 | 353 | 39 | 8258 |
| | signaling | | | | | | | |
| KEGG | Human cytomegalovirus | KEGG: 05163 | 1.90E−19 | 18.72154419 | 242 | 353 | 50 | 8258 |
| | infection | | | | | | | |
| KEGG | MAPK signaling pathway | KEGG: 04010 | 3.48E−19 | 18.45808527 | 286 | 353 | 54 | 8258 |
| KEGG | Cholinergic synapse | KEGG: 04725 | 1.00E−17 | 16.99899228 | 110 | 353 | 33 | 8258 |
| KEGG | Amphetamine addiction | KEGG: 05031 | 1.45E−17 | 16.83943344 | 64 | 353 | 26 | 8258 |
| KEGG | cAMP signaling pathway | KEGG: 04024 | 1.86E−16 | 15.73156372 | 209 | 353 | 43 | 8258 |
| KEGG | GABAergic synapse | KEGG: 04727 | 1.18E−15 | 14.92853643 | 88 | 353 | 28 | 8258 |
| KEGG | Amyotrophic lateral sclerosis | KEGG: 05014 | 3.45E−15 | 14.46197358 | 52 | 353 | 22 | 8258 |
| | (ALS) | | | | | | | |
| KEGG | Kaposi sarcoma-associated | KEGG: 05167 | 3.61E−15 | 14.44309188 | 205 | 353 | 41 | 8258 |
| | herpesvirus infection | | | | | | | |
| KEGG | Long-term depression | KEGG: 04730 | 5.85E−15 | 14.23280157 | 59 | 353 | 23 | 8258 |
| KEGG | Relaxin signaling pathway | KEGG: 04926 | 1.12E−14 | 13.95216121 | 127 | 353 | 32 | 8258 |
| KEGG | GnRH signaling pathway | KEGG: 04912 | 1.31E−14 | 13.88306132 | 88 | 353 | 27 | 8258 |
| KEGG | Human T-cell leukemia virus 1 | KEGG: C6166 | 1.56E−14 | 13.80611057 | 234 | 353 | 43 | 8258 |
| | infection | | | | | | | |
| KEGG | Nicotine addiction | KEGG: C6033 | 3.61E−14 | 13.4430532 | 40 | 353 | 19 | 8258 |
| KEGG | Sphingolipid signaling | KEGG: 04071 | 1.04E−13 | 12.98290089 | 119 | 353 | 30 | 8258 |
| | pathway | | | | | | | |
| KEGG | Rap1 signaling pathway | KEGG: 04015 | 2.63E−13 | 12.58045075 | 209 | 353 | 39 | 8258 |
| KEGG | Endocrine resistance | KEGG: 01522 | 3.40E−13 | 12.46840632 | 91 | 353 | 26 | 8258 |
| KEGG | Calcium signaling pathway | KEGG: 04020 | 3.44E−13 | 12.46311331 | 190 | 353 | 37 | 8258 |
| KEGG | HIF-1 signaling pathway | KEGG: 04066 | 5.80E−13 | 12.23654418 | 109 | 353 | 28 | 8258 |
| KEGG | EGFR tyrosine kinase inhibitor | KEGG: 01521 | 3.55E−12 | 11.44944815 | 76 | 353 | 23 | 8258 |
| | resistance | | | | | | | |
| KEGG | Apoptosis | KEGG: 04210 | 4.11E−12 | 11.38658517 | 135 | 353 | 30 | 8258 |
| KEGG | Ras signaling pathway | KEGG: 04014 | 5.26E−12 | 11.27935174 | 228 | 353 | 39 | 8258 |
| KEGG | Serotonergic synapse | KEGG: 04726 | 1.00E−11 | 10.99929159 | 130 | 353 | 29 | 8258 |
| KEGG | Apelin signaling pathway | KEGG: 04371 | 2.30E−11 | 10.63803092 | 134 | 353 | 29 | 8258 |
| KEGG | AGE-RAGE signaling | KEGG: 04933 | 2.68E−11 | 10.57261882 | 99 | 353 | 25 | 8258 |
| | pathway in diabetic | | | | | | | |
| | complications | | | | | | | |
| KEGG | TNF signaling pathway | KEGG: 04668 | 3.75E−11 | 10.42580806 | 109 | 353 | 26 | 8258 |
| KEGG | ErbB signaling pathway | KEGG: 04012 | 1.52E−10 | 9.818243767 | 81 | 353 | 22 | 8258 |
| KEGG | Synaptic vesicle cycle | KEGG: 04721 | 1.94E−10 | 9.711985713 | 74 | 353 | 21 | 8258 |
| KEGG | FoxO signaling pathway | KEGG: 04068 | 2.06E−10 | 9.686491524 | 126 | 353 | 27 | 8258 |
| KEGG | Human papillomavirus | KEGG: C6165 | 3.35E−10 | 9.474724308 | 346 | 353 | 46 | 8258 |
| | infection | | | | | | | |
| KEGG | Human immunodeficiency | KEGG: C6170 | 3.61E−10 | 9.442493717 | 224 | 353 | 36 | 8258 |
| | virus 1 infection | | | | | | | |
| KEGG | Hepatitis B | KEGG: C6161 | 3.83E−10 | 9.416875182 | 159 | 353 | 30 | 8258 |
| KEGG | Toxoplasmosis | KEGG: C6145 | 1.29E−09 | 8.888990068 | 107 | 353 | 24 | 8258 |
| KEGG | PI3K-Akt signaling pathway | KEGG: 04151 | 1.90E−09 | 8.720472687 | 350 | 353 | 45 | 8258 |
| KEGG | T cell receptor signaling | KEGG: 04660 | 2.02E−09 | 8.695073 | 100 | 353 | 23 | 8258 |
| | pathway | | | | | | | |
| KEGG | Estrogen signaling pathway | KEGG: 04915 | 4.06E−09 | 8.391582848 | 132 | 353 | 26 | 8258 |
| KEGG | Alzheimer disease | KEGG: C6010 | 6.41E−09 | 8.192822988 | 166 | 353 | 29 | 8258 |
| KEGG | Morphine addiction | KEGG: C6032 | 7.36E−09 | 8.13314315 | 88 | 353 | 21 | 8258 |
| KEGG | Thyroid hormone signaling | KEGG: 04919 | 7.96E−09 | 8.099187752 | 116 | 353 | 24 | 8258 |
| | pathway | | | | | | | |
| KEGG | Chemokine signaling pathway | KEGG: 04062 | 1.11E−08 | 7.955970993 | 192 | 353 | 31 | 8258 |
| KEGG | Focal adhesion | KEGG: 04510 | 1.45E−08 | 7.838277399 | 194 | 353 | 31 | 8258 |
| KEGG | Pertussis | KEGG: C6133 | 1.99E−08 | 7.700730149 | 75 | 353 | 19 | 8258 |
| KEGG | Tuberculosis | KEGG: C6152 | 2.40E−08 | 7.619316696 | 175 | 353 | 29 | 8258 |
| KEGG | Longevity regulating pathway - | KEGG: 04213 | 2.71E−08 | 7.567598113 | 60 | 353 | 17 | 8258 |
| | multiple species | | | | | | | |
| KEGG | Gap junction | KEGG: 04540 | 2.78E−08 | 7.556654802 | 85 | 353 | 20 | 8258 |
| KEGG | Parathyroid hormone synthesis, | KEGG: 04928 | 3.19E−08 | 7.496077731 | 104 | 353 | 22 | 8258 |
| | secretion and action | | | | | | | |
| KEGG | Insulin resistance | KEGG: 04931 | 3.19E−08 | 7.496077731 | 104 | 353 | 22 | 8258 |
| KEGG | Melanogenesis | KEGG: 04916 | 4.22E−08 | 7.374261208 | 96 | 353 | 21 | 8258 |
| KEGG | Longevity regulating pathway | KEGG: 04211 | 5.40E−08 | 7.267473592 | 88 | 353 | 20 | 8258 |
| KEGG | Cocaine addiction | KEGG: C6030 | 6.77E−08 | 7.169557467 | 48 | 353 | 15 | 8258 |
| KEGG | VEGF signaling pathway | KEGG: 04370 | 8.02E−08 | 7.095780802 | 56 | 353 | 16 | 8258 |
| KEGG | Chagas disease (American | KEGG: C6142 | 1.14E−07 | 6.941753568 | 101 | 353 | 21 | 8258 |
| | trypanosomiasis) | | | | | | | |
| KEGG | Inflammatory mediator | KEGG: 04750 | 1.40E−07 | 6.853967098 | 122 | 353 | 23 | 8258 |
| | regulation of TRP channels | | | | | | | |
| KEGG | Fluid shear stress and | KEGG: C5418 | 3.34E−07 | 6.475789928 | 138 | 353 | 24 | 8258 |
| | atherosclerosis | | | | | | | |
| KEGG | Neurotrophin signaling | KEGG: 04722 | 4.01E−07 | 6.39648433 | 118 | 353 | 22 | 8258 |
| | pathway | | | | | | | |
| KEGG | Platelet activation | KEGG: 04611 | 4.73E−07 | 6.324777138 | 119 | 353 | 22 | 8258 |
| KEGG | C-type lectin receptor signaling | KEGG: 04625 | 4.94E−07 | 6.306662102 | 109 | 353 | 21 | 8258 |
| | pathway | | | | | | | |
| KEGG | Type II diabetes mellitus | KEGG: 04930 | 5.16E−07 | 6.287627544 | 47 | 353 | 14 | 8258 |
| KEGG | Axon guidance | KEGG: 04360 | 8.62E−07 | 6.064334693 | 179 | 353 | 27 | 8258 |
| KEGG | Renin secretion | KEGG: 04924 | 9.26E−07 | 6.033290454 | 74 | 353 | 17 | 8258 |
| KEGG | Glucagon signaling pathway | KEGG: 04922 | 1.00552E−06 | 5.997608854 | 103 | 353 | 20 | 8258 |
| KEGG | Aldosterone synthesis and | KEGG: 04925 | 2.90034E−06 | 5.53755066 | 99 | 353 | 19 | 8258 |
| | secretion | | | | | | | |
| KEGG | Adipocytokine signaling | KEGG: 04920 | 3.42631E−06 | 5.465173601 | 71 | 353 | 16 | 8258 |
| | pathway | | | | | | | |
| KEGG | Autophagy - animal | KEGG: 04140 | 3.45047E−06 | 5.462122323 | 132 | 353 | 22 | 8258 |
| KEGG | cGMP-PKG signaling pathway | KEGG: 04022 | 3.66505E−06 | 5.435920285 | 167 | 353 | 25 | 8258 |
| KEGG | Cushing syndrome | KEGG: 04934 | 4.04879E−06 | 5.392675155 | 156 | 353 | 24 | 8258 |
| KEGG | Apoptosis - multiple species | KEGG: 04215 | 4.43931E−06 | 5.352684372 | 32 | 353 | 11 | 8258 |
| KEGG | Phospholipase D signaling | KEGG: 04072 | 4.98255E−06 | 5.302548397 | 146 | 353 | 23 | 8258 |
| | pathway | | | | | | | |
| KEGG | Insulin secretion | KEGG: 04911 | 5.77972E−06 | 5.238093015 | 83 | 353 | 17 | 8258 |
| KEGG | Hepatitis C | KEGG: C6160 | 5.89899E−06 | 5.229222068 | 159 | 353 | 24 | 8258 |
| KEGG | Wnt signaling pathway | KEGG: 04310 | 6.6725E−06 | 5.175711716 | 160 | 353 | 24 | 8258 |
| KEGG | GnRH secretion | KEGG: 04929 | 9.9678E−06 | 5.001400511 | 58 | 353 | 14 | 8258 |
| KEGG | Th17 cell differentiation | KEGG: 04659 | 2.21C62E−05 | 4.655486369 | 101 | 353 | 18 | 8258 |
| KEGG | Amoebiasis | KEGG: C6146 | 3.00739E−05 | 4.521810727 | 103 | 353 | 18 | 8258 |
| KEGG | Fc epsilon RI signaling | KEGG: 04664 | 4.56282E−05 | 4.340766824 | 65 | 353 | 14 | 8258 |
| | pathway | | | | | | | |
| KEGG | Measles | KEGG: C6162 | 5.77287E−05 | 4.238607925 | 142 | 353 | 21 | 8258 |
| KEGG | Acute myeloid leukemia | KEGG: C6221 | 8.20239E−05 | 4.086059391 | 68 | 353 | 14 | 8258 |
| KEGG | Leukocyte transendothelial | KEGG: 04670 | 9.52796E−05 | 4.021000001 | 111 | 353 | 18 | 8258 |
| | migration | | | | | | | |
| KEGG | Prolactin signaling pathway | KEGG: 04917 | 0.000118991 | 3.924484201 | 70 | 353 | 14 | 8258 |
| KEGG | Rheumatoid arthritis | KEGG: C6323 | 0.000191842 | 3.717057211 | 83 | 353 | 15 | 8258 |
| KEGG | Yersinia infection | KEGG: C6135 | 0.000270227 | 3.568271749 | 119 | 353 | 18 | 8258 |
| KEGG | Huntington disease | KEGG: C6016 | 0.000339492 | 3.469170972 | 184 | 353 | 23 | 8258 |
| KEGG | B cell receptor signaling | KEGG: 04662 | 0.0C0393869 | 3.404648301 | 77 | 353 | 14 | 8258 |
| | pathway | | | | | | | |
| KEGG | mTOR signaling pathway | KEGG: 04150 | 0.000408037 | 3.389300251 | 147 | 353 | 20 | 8258 |
| KEGG | Epstein-Barr virus infection | KEGG: C6169 | 0.0C0563614 | 3.249018568 | 217 | 353 | 25 | 8258 |
| KEGG | JAK-STAT signaling pathway | KEGG: 04630 | 0.0C0565041 | 3.247920354 | 163 | 353 | 21 | 8258 |
| KEGG | Endocrine and other factor- | KEGG: 04961 | 0.0C0581406 | 3.235520688 | 59 | 353 | 12 | 8258 |
| | regulated calcium reabsorption | | | | | | | |
| KEGG | Influenza A | KEGG: C6164 | 0.0C0623064 | 3.205467094 | 164 | 353 | 21 | 8258 |
| KEGG | Prion diseases | KEGG: C6020 | 0.0C0904325 | 3.043675691 | 34 | 353 | 9 | 8258 |
| KEGG | Mitophagy - animal | KEGG: 04137 | 0.001187492 | 2.925369375 | 63 | 353 | 12 | 8258 |
| KEGG | Platinum drug resistance | KEGG: 01524 | 0.001296358 | 2.887275093 | 74 | 353 | 13 | 8258 |
| KEGG | Insulin signaling pathway | KEGG: 04910 | 0.001342623 | 2.872045787 | 133 | 353 | 18 | 8258 |
| KEGG | Toll-like receptor signaling | KEGG: 04620 | 0.001413876 | 2.849588527 | 97 | 353 | 15 | 8258 |
| | pathway | | | | | | | |
| KEGG | Necroptosis | KEGG: 04217 | 0.001444871 | 2.840170838 | 173 | 353 | 21 | 8258 |
| KEGG | Viral protein interaction with | KEGG: 04061 | 0.002059497 | 2.686238742 | 100 | 353 | 15 | 8258 |
| | cytokine and cytokine receptor | | | | | | | |
| KEGG | Sphingolipid metabolism | KEGG: C0600 | 0.0023343 | 2.631843369 | 47 | 353 | 10 | 8258 |
| KEGG | Alcoholism | KEGG: C6034 | 0.003391669 | 2.469586534 | 197 | 353 | 22 | 8258 |
| KEGG | Cellular senescence | KEGG: 04218 | 0.003667168 | 2.435669162 | 170 | 353 | 20 | 8258 |
| KEGG | Cytokine-cytokine receptor | KEGG: 04060 | 0.004413466 | 2.355220235 | 290 | 353 | 28 | 8258 |
| | interaction | | | | | | | |
| KEGG | NF-kappa B signaling pathway | KEGG: 04064 | 0.004668349 | 2.33083669 | 107 | 353 | 15 | 8258 |
| KEGG | Natural killer cell mediated | KEGG: 04650 | 0.006477427 | 2.188597493 | 110 | 353 | 15 | 8258 |
| | cytotoxicity | | | | | | | |
| KEGG | Neuroactive ligand-receptor | KEGG: 04080 | 0.008056569 | 2.09384989 | 348 | 353 | 31 | 8258 |
| | interaction | | | | | | | |
| KEGG | Salivary secretion | KEGG: 04970 | 0.008363949 | 2.077588619 | 76 | 353 | 12 | 8258 |
| KEGG | Tight junction | KEGG: 04530 | 0.008379054 | 2.076805001 | 166 | 353 | 19 | 8258 |
| KEGG | IL-17 signaling pathway | KEGG: 04657 | 0.008652229 | 2.06287201 | 88 | 353 | 13 | 8258 |
| KEGG | Cortisol synthesis and | KEGG: 04927 | 0.015129502 | 1.820175368 | 69 | 353 | 11 | 8258 |
| | secretion | | | | | | | |
| KEGG | Hematopoietic cell lineage | KEGG: 04640 | 0.017217632 | 1.764026569 | 94 | 353 | 13 | 8258 |
| KEGG | Lysosome | KEGG: 04142 | 0.021280786 | 1.672012345 | 122 | 353 | 15 | 8258 |
| KEGG | Thyroid hormone synthesis | KEGG: 04918 | 0.022353456 | 1.650655329 | 72 | 353 | 11 | 8258 |
| KEGG | Th1 and Th2 cell | KEGG: 04658 | 0.028065928 | 1.551820594 | 86 | 353 | 12 | 8258 |
| | differentiation | | | | | | | |
| KEGG | Fc gamma R-mediated | KEGG: 04666 | 0.028065928 | 1.551820594 | 86 | 353 | 12 | 8258 |
| | phagocytosis | | | | | | | |
| KEGG | Cell adhesion molecules | KEGG: 04514 | 0.044707655 | 1.349618107 | 159 | 353 | 17 | 8258 |
| | (CAMs) | | | | | | | |

TABLE 16
From TTPB_0400_final document, Differentially Expressed Genes - GProfiler Results - DIV08

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Glutamatergic synapse | KEGG: 047 | 1.19E−24 | 23.92478 | 112 | 314 | 38 | 8258 |
| KEGG | Long-term potentiation | KEGG: 047 | 5.40E−18 | 17.26783 | 62 | 314 | 25 | 8258 |
| KEGG | MAPK signaling pathway | KEGG: 040 | 1.33E−17 | 16.87718 | 286 | 314 | 49 | 8258 |
| KEGG | Retrograde endocannabinoid | KEGG: 047 | 2.13E−16 | 15.67226 | 143 | 314 | 34 | 8258 |
| | signaling | | | | | | | |
| KEGG | Dopaminergic synapse | KEGG: 047 | 3.52E−16 | 15.45308 | 127 | 314 | 32 | 8258 |
| KEGG | Human cytomegalovirus infection | KEGG: 051 | 6.96E−16 | 15.15717 | 242 | 314 | 43 | 8258 |
| KEGG | Amphetamine addiction | KEGG: 050 | 3.74E−15 | 14.4266 | 64 | 314 | 23 | 8258 |
| KEGG | Amyotrophic lateral sclerosis (ALS) | KEGG: 050 | 5.47E−15 | 14.26184 | 52 | 314 | 21 | 8258 |
| KEGG | Circadian entrainment | KEGG: 047 | 8.02E−15 | 14.09604 | 96 | 314 | 27 | 8258 |
| KEGG | Oxytocin signaling pathway | KEGG: 049 | 3.61E−14 | 13.44295 | 147 | 314 | 32 | 8258 |
| KEGG | Nicotine addiction | KEGG: 050 | 9.60E−14 | 13.01792 | 40 | 314 | 18 | 8258 |
| KEGG | Calcium signaling pathway | KEGG: 040 | 3.32E−13 | 12.479 | 190 | 314 | 35 | 8258 |
| KEGG | GABAergic synapse | KEGG: 047 | 9.63E−13 | 12.01636 | 88 | 314 | 24 | 8258 |
| KEGG | cAMP signaling pathway | KEGG: 040 | 6.74E−12 | 11.17102 | 209 | 314 | 35 | 8258 |
| KEGG | GnRH signaling pathway | KEGG: 049 | 9.69E−12 | 11.01368 | 88 | 314 | 23 | 8258 |
| KEGG | Cholinergic synapse | KEGG: 047 | 2.55E−11 | 10.5943 | 110 | 314 | 25 | 8258 |
| KEGG | Human T-cell leukemia virus 1 | KEGG: 051 | 4.16E−11 | 10.38073 | 234 | 314 | 36 | 8258 |
| | infection | | | | | | | |
| KEGG | AGE-RAGE signaling pathway in | KEGG: 049 | 1.46E−10 | 9.836225 | 99 | 314 | 23 | 8258 |
| | diabetic complications | | | | | | | |
| KEGG | HIF-1 signaling pathway | KEGG: 040 | 1.63E−10 | 9.786505 | 109 | 314 | 24 | 8258 |
| KEGG | Sphingolipid signaling pathway | KEGG: 040 | 1.70E−10 | 9.770493 | 119 | 314 | 25 | 8258 |
| KEGG | Rap1 signaling pathway | KEGG: 040 | 2.03E−10 | 9.692007 | 209 | 314 | 33 | 8258 |
| KEGG | Long-term depression | KEGG: 047 | 2.84E−10 | 9.547018 | 59 | 314 | 18 | 8258 |
| KEGG | Relaxin signaling pathway | KEGG: 049 | 7.87E−10 | 9.10428 | 127 | 314 | 25 | 8258 |
| KEGG | TNF signaling pathway | KEGG: 0461 | 1.24E−09 | 8.905785 | 109 | 314 | 23 | 8258 |
| KEGG | Endocrine resistance | KEGG: 015 | 1.63E−09 | 8.787079 | 91 | 314 | 21 | 8258 |
| KEGG | Apoptosis | KEGG: 042 | 3.23E−09 | 8.491416 | 135 | 314 | 25 | 8258 |
| KEGG | EGFR tyrosine kinase inhibitor | KEGG: 015 | 3.46E−09 | 8.46085 | 76 | 314 | 19 | 8258 |
| | resistance | | | | | | | |
| KEGG | FoxO signaling pathway | KEGG: 040 | 4.37E−09 | 8.359213 | 126 | 314 | 24 | 8258 |
| KEGG | Morphine addiction | KEGG: 050 | 6.81E−09 | 8.166803 | 88 | 314 | 20 | 8258 |
| KEGG | Ras signaling pathway | KEGG: 040 | 1.11E−08 | 7.956248 | 228 | 314 | 32 | 8258 |
| KEGG | Alzheimer disease | KEGG: 050 | 1.16E−08 | 7.934656 | 166 | 314 | 27 | 8258 |
| KEGG | Apelin signaling pathway | KEGG: 043 | 1.69E−08 | 7.773018 | 134 | 314 | 24 | 8258 |
| KEGG | Human immunodeficiency virus 1 | KEGG: 051 | 3.17E−08 | 7.499391 | 224 | 314 | 31 | 8258 |
| | infection | | | | | | | |
| KEGG | ErbB signaling pathway | KEGG: 040 | 9.34E−08 | 7.029583 | 81 | 314 | 18 | 8258 |
| KEGG | Chagas disease (American | KEGG: 051 | 9.44E−08 | 7.025135 | 101 | 314 | 20 | 8258 |
| | trypanosomiasis) | | | | | | | |
| KEGG | PI3K-Akt signaling pathway | KEGG: 041 | 1.06E−07 | 6.975677 | 350 | 314 | 39 | 8258 |
| KEGG | Hepatitis B | KEGG: 051 | 1.23E−07 | 6.910816 | 159 | 314 | 25 | 8258 |
| KEGG | Synaptic vesicle cycle | KEGG: 047 | 1.60E−07 | 6.794829 | 74 | 314 | 17 | 8258 |
| KEGG | Fluid shear stress and atherosclerosis | KEGG: 054 | 1.81E−07 | 6.743085 | 138 | 314 | 23 | 8258 |
| KEGG | Serotonergic synapse | KEGG: 047 | 3.07E−07 | 6.512742 | 130 | 314 | 22 | 8258 |
| KEGG | Parathyroid hormone synthesis, | KEGG: 049 | 1.03E−06 | 5.986829 | 104 | 314 | 19 | 8258 |
| | secretion and action | | | | | | | |
| KEGG | VEGF signaling pathway | KEGG: 043 | 1.44E−06 | 5.842926 | 56 | 314 | 14 | 8258 |
| KEGG | Chemokine signaling pathway | KEGG: 040 | 1.47E−06 | 5.831823 | 192 | 314 | 26 | 8258 |
| KEGG | Gap junction | KEGG: 045 | 1.53E−06 | 5.81664 | 85 | 314 | 17 | 8258 |
| KEGG | Pertussis | KEGG: 051 | 1.54E−06 | 5.81125 | 75 | 314 | 16 | 8258 |
| KEGG | Inflammatory mediator regulation of | KEGG: 047 | 2.88E−06 | 5.540654 | 122 | 314 | 20 | 8258 |
| | TRP channels | | | | | | | |
| KEGG | T cell receptor signaling pathway | KEGG: 046 | 3.25E−06 | 5.488352 | 100 | 314 | 18 | 8258 |
| KEGG | Insulin resistance | KEGG: 049 | 6.12E−06 | 5.212987 | 104 | 314 | 18 | 8258 |
| KEGG | Toxoplasmosis | KEGG: 051 | 9.65E−06 | 5.01561 | 107 | 314 | 18 | 8258 |
| KEGG | Human papillomavirus infection | KEGG: 051 | 9.81E−06 | 5.008265 | 346 | 314 | 35 | 8258 |
| KEGG | Type II diabetes mellitus | KEGG: 049 | 1.24E−05 | 4.90695 | 47 | 314 | 12 | 8258 |
| KEGG | Sphingolipid metabolism | KEGG: 006 | 1.24E−05 | 4.90695 | 47 | 314 | 12 | 8258 |
| KEGG | C-type lectin receptor signaling | KEGG: 046 | 1.29E−05 | 4.888116 | 109 | 314 | 18 | 8258 |
| | pathway | | | | | | | |
| KEGG | Apoptosis - multiple species | KEGG: 042 | 1.78E−05 | 4.750261 | 32 | 314 | 10 | 8258 |
| KEGG | Tuberculosis | KEGG: 051 | 1.82E−05 | 4.740376 | 175 | 314 | 23 | 8258 |
| KEGG | Glucagon signaling pathway | KEGG: 049 | 2.94E−05 | 4.530983 | 103 | 314 | 17 | 8258 |
| KEGG | Focal adhesion | KEGG: 045 | 3.08E−05 | 4.511081 | 194 | 314 | 24 | 8258 |
| KEGG | Adipocytokine signaling pathway | KEGG: 049 | 3.55E−05 | 4.449608 | 71 | 314 | 14 | 8258 |
| KEGG | Huntington disease | KEGG: 050 | 4.56E−05 | 4.341371 | 184 | 314 | 23 | 8258 |
| KEGG | Estrogen signaling pathway | KEGG: 049 | 5.36E−05 | 4.271061 | 132 | 314 | 19 | 8258 |
| KEGG | Hepatitis C | KEGG: 051 | 5.84E−05 | 4.23354 | 159 | 314 | 21 | 8258 |
| KEGG | Aldosterone synthesis and secretion | KEGG: 049 | 8.91E−05 | 4.050105 | 99 | 314 | 16 | 8258 |
| KEGG | Longevity regulating pathway | KEGG: 042 | 9.77E−05 | 4.009961 | 88 | 314 | 15 | 8258 |
| KEGG | Influenza A | KEGG: 051 | 9.83E−05 | 4.007296 | 164 | 314 | 21 | 8258 |
| KEGG | Th17 cell differentiation | KEGG: 046 | 0.000118 | 3.929475 | 101 | 314 | 16 | 8258 |
| KEGG | cGMP-PKG signaling pathway | KEGG: 040 | 0.000133 | 3.876228 | 167 | 314 | 21 | 8258 |
| KEGG | Cocaine addiction | KEGG: 050 | 0.000137 | 3.863414 | 48 | 314 | 11 | 8258 |
| KEGG | Thyroid hormone signaling pathway | KEGG: 049 | 0.000167 | 3.777068 | 116 | 314 | 17 | 8258 |
| KEGG | Measles | KEGG: 051 | 0.000168 | 3.775289 | 142 | 314 | 19 | 8258 |
| KEGG | Longevity regulating pathway - | KEGG: 042 | 0.000213 | 3.672333 | 60 | 314 | 12 | 8258 |
| | multiple species | | | | | | | |
| KEGG | Neurotrophin signaling pathway | KEGG: 047 | 0.000213 | 3.671345 | 118 | 314 | 17 | 8258 |
| KEGG | Phospholipase D signaling pathway | KEGG: 040 | 0.000257 | 3.590526 | 146 | 314 | 19 | 8258 |
| KEGG | Rheumatoid arthritis | KEGG: 053 | 0.000258 | 3.588043 | 83 | 314 | 14 | 8258 |
| KEGG | Insulin secretion | KEGG: 049 | 0.000258 | 3.588043 | 83 | 314 | 14 | 8258 |
| KEGG | Wnt signaling pathway | KEGG: 043 | 0.000263 | 3.579757 | 160 | 314 | 20 | 8258 |
| KEGG | Neuroactive ligand-receptor | KEGG: 040 | 0.0003 | 3.523063 | 348 | 314 | 32 | 8258 |
| | interaction | | | | | | | |
| KEGG | Melanogenesis | KEGG: 049 | 0.000306 | 3.514536 | 96 | 314 | 15 | 8258 |
| KEGG | Prion diseases | KEGG: 050 | 0.000355 | 3.449386 | 34 | 314 | 9 | 8258 |
| KEGG | Renin secretion | KEGG: 049 | 0.000371 | 3.43101 | 74 | 314 | 13 | 8258 |
| KEGG | Viral protein interaction with | KEGG: 040 | 0.000516 | 3.287338 | 100 | 314 | 15 | 8258 |
| | cytokine and cytokine receptor | | | | | | | |
| KEGG | GnRH secretion | KEGG: 049 | 0.000982 | 3.007867 | 58 | 314 | 11 | 8258 |
| KEGG | Yersinia infection | KEGG: 051 | 0.001063 | 2.973313 | 119 | 314 | 16 | 8258 |
| KEGG | Platelet activation | KEGG: 046 | 0.001063 | 2.973313 | 119 | 314 | 16 | 8258 |
| KEGG | Endocrine and other factor-regulated | KEGG: 049 | 0.001167 | 2.933019 | 59 | 314 | 11 | 8258 |
| | calcium reabsorption | | | | | | | |
| KEGG | Axon guidance | KEGG: 043 | 0.001474 | 2.831554 | 179 | 314 | 20 | 8258 |
| KEGG | Mitophagy - animal | KEGG: 041 | 0.002241 | 2.649565 | 63 | 314 | 11 | 8258 |
| KEGG | Cushing syndrome | KEGG: 049 | 0.002574 | 2.589395 | 156 | 314 | 18 | 8258 |
| KEGG | Epstein-Barr virus infection | KEGG: 051 | 0.002587 | 2.587195 | 217 | 314 | 22 | 8258 |
| KEGG | Autophagy - animal | KEGG: 041 | 0.003977 | 2.400434 | 132 | 314 | 16 | 8258 |
| KEGG | Natural killer cell mediated | KEGG: 046 | 0.007097 | 2.148908 | 110 | 314 | 14 | 8258 |
| | cytotoxicity | | | | | | | |
| KEGG | p53 signaling pathway | KEGG: 041 | 0.007103 | 2.148553 | 71 | 314 | 11 | 8258 |
| KEGG | Leukocyte transendothelial | KEGG: 046 | 0.007848 | 2.105243 | 111 | 314 | 14 | 8258 |
| | migration | | | | | | | |
| KEGG | Cellular senescence | KEGG: 042 | 0.008132 | 2.089794 | 170 | 314 | 18 | 8258 |
| KEGG | Necroptosis | KEGG: 042 | 0.010215 | 1.990755 | 173 | 314 | 18 | 8258 |
| KEGG | Platinum drag resistance | KEGG: 015 | 0.010474 | 1.979882 | 74 | 314 | 11 | 8258 |
| KEGG | IL-17 signaling pathway | KEGG: 046 | 0.012009 | 1.920506 | 88 | 314 | 12 | 8258 |
| KEGG | Salivary secretion | KEGG: 049 | 0.013412 | 1.87252 | 76 | 314 | 11 | 8258 |
| KEGG | Amoebiasis | KEGG: 051 | 0.014055 | 1.852181 | 103 | 314 | 13 | 8258 |
| KEGG | Fc epsilon RI signaling pathway | KEGG: 046 | 0.016163 | 1.79147 | 65 | 314 | 10 | 8258 |
| KEGG | Lysosome | KEGG: 041 | 0.021902 | 1.659515 | 122 | 314 | 14 | 8258 |
| KEGG | Leishmaniasis | KEGG: 051 | 0.023732 | 1.624666 | 68 | 314 | 10 | 8258 |
| KEGG | Cytokine-cytokine receptor | KEGG: 040 | 0.028383 | 1.546941 | 290 | 314 | 24 | 8258 |
| | interaction | | | | | | | |
| KEGG | Prolactin signaling pathway | KEGG: 049 | 0.030275 | 1.518923 | 70 | 314 | 10 | 8258 |
| KEGG | African trypanosomiasis | KEGG: 051 | 0.030547 | 1.51503 | 35 | 314 | 7 | 8258 |
| KEGG | Cell adhesion molecules (CAMs) | KEGG: 045 | 0.036437 | 1.438457 | 159 | 314 | 16 | 8258 |
| KEGG | Th1 and Th2 cell differentiation | KEGG: 046 | 0.04085 | 1.38881 | 86 | 314 | 11 | 8258 |
| KEGG | mTOR signaling pathway | KEGG: 041 | 0.047852 | 1.320103 | 147 | 314 | 15 | 8258 |

TABLE 17
From TTPB_0400_final document, Differentially Expressed Genes - GProfiler Results - DIV11

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Glutamatergic synapse | KEGG: 047 | 4.72E−23 | 22.32599 | 112 | 300 | 36 | 8258 |
| KEGG | Human cytomegalovirus infection | KEGG: 051 | 6.42E−21 | 20.19262 | 242 | 300 | 48 | 8258 |
| KEGG | Dopaminergic synapse | KEGG: 047 | 8.10E−19 | 18.09146 | 127 | 300 | 34 | 8258 |
| KEGG | Long-term potentiation | KEGG: 047 | 1.75E−18 | 17.75725 | 62 | 300 | 25 | 8258 |
| KEGG | Oxytocin signaling pathway | KEGG: 049 | 1.38E−17 | 16.86109 | 147 | 300 | 35 | 8258 |
| KEGG | Retrograde endocannabinoid | KEGG: 047 | 4.96E−17 | 16.3046 | 143 | 300 | 34 | 8258 |
| | signaling | | | | | | | |
| KEGG | Circadian entrainment | KEGG: 047 | 2.48E−15 | 14.60498 | 96 | 300 | 27 | 8258 |
| KEGG | Long-term depression | KEGG: 047 | 2.78E−15 | 14.55635 | 59 | 300 | 22 | 8258 |
| KEGG | cAMP signaling pathway | KEGG: 040 | 6.53E−15 | 14.18479 | 209 | 300 | 38 | 8258 |
| KEGG | Cholinergic synapse | KEGG: 047 | 1.12E−14 | 13.9526 | 110 | 300 | 28 | 8258 |
| KEGG | MAPK signaling pathway | KEGG: 040 | 1.35E−14 | 13.86962 | 286 | 300 | 44 | 8258 |
| KEGG | Amyotrophic lateral sclerosis (ALS) | KEGG: 050 | 6.85E−13 | 12.16441 | 52 | 300 | 19 | 8258 |
| KEGG | Sphingolipid signaling pathway | KEGG: 040 | 9.05E−13 | 12.04316 | 119 | 300 | 27 | 8258 |
| KEGG | Nicotine addiction | KEGG: 050 | 9.41E−13 | 12.02642 | 40 | 300 | 17 | 8258 |
| KEGG | Rap1 signaling pathway | KEGG: 040 | 1.67E−12 | 11.77688 | 209 | 300 | 35 | 8258 |
| KEGG | Calcium signaling pathway | KEGG: 040 | 3.36E−12 | 11.47356 | 190 | 300 | 33 | 8258 |
| KEGG | GABAergic synapse | KEGG: 047 | 3.63E−12 | 11.43982 | 88 | 300 | 23 | 8258 |
| KEGG | GnRH signaling pathway | KEGG: 049 | 3.63E−12 | 11.43982 | 88 | 300 | 23 | 8258 |
| KEGG | Amphetamine addiction | KEGG: 050 | 4.18E−12 | 11.37878 | 64 | 300 | 20 | 8258 |
| KEGG | HIF-1 signaling pathway | KEGG: 040 | 7.14E−12 | 11.14619 | 109 | 300 | 25 | 8258 |
| KEGG | Endocrine resistance | KEGG: 015 | 7.98E−12 | 11.09812 | 91 | 300 | 23 | 8258 |
| KEGG | PI3K-Akt signaling pathway | KEGG: 041 | 2.48E−11 | 10.60561 | 350 | 300 | 44 | 8258 |
| KEGG | Human T-cell leukemia virus 1 | KEGG: 051 | 5.45E−11 | 10.26321 | 234 | 300 | 35 | 8258 |
| | infection | | | | | | | |
| KEGG | AGE-RAGE signaling pathway in | KEGG: 049 | 5.57E−11 | 10.25433 | 99 | 300 | 23 | 8258 |
| | diabetic complications | | | | | | | |
| KEGG | Serotonergic synapse | KEGG: 047 | 6.90E−11 | 10.1611 | 130 | 300 | 26 | 8258 |
| KEGG | Human papillomavirus infection | KEGG: 051 | 7.16E−11 | 10.14482 | 346 | 300 | 43 | 8258 |
| KEGG | Relaxin signaling pathway | KEGG: 049 | 2.84E−10 | 9.545969 | 127 | 300 | 25 | 8258 |
| KEGG | Axon guidance | KEGG: 043 | 8.12E−10 | 9.09022 | 179 | 300 | 29 | 8258 |
| KEGG | Apelin signaling pathway | KEGG: 043 | 9.96E−10 | 9.001906 | 134 | 300 | 25 | 8258 |
| KEGG | Ras signaling pathway | KEGG: 040 | 3.31E−09 | 8.47964 | 228 | 300 | 32 | 8258 |
| KEGG | Alzheimer disease | KEGG: 050 | 4.06E−09 | 8.391988 | 166 | 300 | 27 | 8258 |
| KEGG | VEGF signaling pathway | KEGG: 043 | 7.32E−09 | 8.135448 | 56 | 300 | 16 | 8258 |
| KEGG | Apoptosis | KEGG: 042 | 7.64E−09 | 8.116814 | 135 | 300 | 24 | 8258 |
| KEGG | Human immunodeficiency virus 1 | KEGG: 051 | 9.93E−09 | 8.003214 | 224 | 300 | 31 | 8258 |
| | infection | | | | | | | |
| KEGG | Tuberculosis | KEGG: 051 | 1.42E−08 | 7.848392 | 175 | 300 | 27 | 8258 |
| KEGG | Neurotrophin signaling pathway | KEGG: 047 | 1.87E−08 | 7.728887 | 118 | 300 | 22 | 8258 |
| KEGG | cGMP-PKG signaling pathway | KEGG: 040 | 2.58E−08 | 7.587816 | 167 | 300 | 26 | 8258 |
| KEGG | TNF signaling pathway | KEGG: 046 | 2.59E−08 | 7.58628 | 109 | 300 | 21 | 8258 |
| KEGG | C-type lectin receptor signaling | KEGG: 046 | 2.59E−08 | 7.58628 | 109 | 300 | 21 | 8258 |
| | pathway | | | | | | | |
| KEGG | Chagas disease (American | KEGG: 051 | 4.20E−08 | 7.376294 | 101 | 300 | 20 | 8258 |
| | trypanosomiasis) | | | | | | | |
| KEGG | FoxO signaling pathway | KEGG: 040 | 6.99E−08 | 7.155352 | 126 | 300 | 22 | 8258 |
| KEGG | Fluid shear stress and | KEGG: 054 | 7.37E−08 | 7.132744 | 138 | 300 | 23 | 8258 |
| | atherosclerosis | | | | | | | |
| KEGG | EGER tyrosine kinase inhibitor | KEGG: 015 | 1.24E−07 | 6.9077 | 76 | 300 | 17 | 8258 |
| | resistance | | | | | | | |
| KEGG | Focal adhesion | KEGG: 045 | 1.53E−07 | 6.816314 | 194 | 300 | 27 | 8258 |
| KEGG | Estrogen signaling pathway | KEGG: 049 | 1.75E−07 | 6.755991 | 132 | 300 | 22 | 8258 |
| KEGG | Hepatitis B | KEGG: 051 | 2.49E−07 | 6.604079 | 159 | 300 | 24 | 8258 |
| KEGG | ErbB signaling pathway | KEGG: 040 | 3.52E−07 | 6.452894 | 81 | 300 | 17 | 8258 |
| KEGG | Parathyroid hormone synthesis, | KEGG: 049 | 4.85E−07 | 6.314196 | 104 | 300 | 19 | 8258 |
| | secretion and action | | | | | | | |
| KEGG | Chemokine signaling pathway | KEGG: 040 | 5.65E−07 | 6.247905 | 192 | 300 | 26 | 8258 |
| KEGG | Type II diabetes mellitus | KEGG: 049 | 7.25E−07 | 6.139497 | 47 | 300 | 13 | 8258 |
| KEGG | Yersinia infection | KEGG: 051 | 8.53E−07 | 6.06908 | 119 | 300 | 20 | 8258 |
| KEGG | Platelet activation | KEGG: 046 | 8.53E−07 | 6.06908 | 119 | 300 | 20 | 8258 |
| KEGG | Morphine addiction | KEGG: 050 | 1.34E−06 | 5.874264 | 88 | 300 | 17 | 8258 |
| KEGG | T cell receptor signaling pathway | KEGG: 046 | 1.6E−06 | 5.797062 | 100 | 300 | 18 | 8258 |
| KEGG | Glucagon signaling pathway | KEGG: 049 | 2.59E−06 | 5.587047 | 103 | 300 | 18 | 8258 |
| KEGG | Thyroid hormone signaling pathway | KEGG: 049 | 3.15E−06 | 5.501662 | 116 | 300 | 19 | 8258 |
| KEGG | Cushing syndrome | KEGG: 049 | 4.23E−06 | 5.373496 | 156 | 300 | 22 | 8258 |
| KEGG | Toxoplasmosis | KEGG: 051 | 4.79E−06 | 5.319314 | 107 | 300 | 18 | 8258 |
| KEGG | Melanogenesis | KEGG: 049 | 5.22E−06 | 5.282683 | 96 | 300 | 17 | 8258 |
| KEGG | Gap junction | KEGG: 045 | 5.32E−06 | 5.274054 | 85 | 300 | 16 | 8258 |
| KEGG | Pertussis | KEGG: 051 | 6.01E−06 | 5.221142 | 75 | 300 | 15 | 8258 |
| KEGG | Hepatitis C | KEGG: 051 | 6.01E−06 | 5.221121 | 159 | 300 | 22 | 8258 |
| KEGG | Phospholipase D signaling pathway | KEGG: 040 | 6.13E−06 | 5.2123 | 146 | 300 | 21 | 8258 |
| KEGG | Wnt signaling pathway | KEGG: 043 | 6.74E−06 | 5.171209 | 160 | 300 | 22 | 8258 |
| KEGG | Inflammatory mediator regulation of | KEGG: 047 | 7.31E−06 | 5.136305 | 122 | 300 | 19 | 8258 |
| | TRP channels | | | | | | | |
| KEGG | Cocaine addiction | KEGG: 050 | 9.67E−06 | 5.014687 | 48 | 300 | 12 | 8258 |
| KEGG | GnRH secretion | KEGG: 049 | 1.13E−05 | 4.945146 | 58 | 300 | 13 | 8258 |
| KEGG | Renin secretion | KEGG: 049 | 3.48E−05 | 4.458874 | 74 | 300 | 14 | 8258 |
| KEGG | Fc epsilon RI signaling pathway | KEGG: 046 | 4.7E−05 | 4.328332 | 65 | 300 | 13 | 8258 |
| KEGG | Longevity regulating pathway | KEGG: 042 | 5.46E−05 | 4.263176 | 88 | 300 | 15 | 8258 |
| KEGG | Longevity regulating pathway - | KEGG: 042 | 0.000131 | 3.881816 | 60 | 300 | 12 | 8258 |
| | multiple species | | | | | | | |
| KEGG | Synaptic vesicle cycle | KEGG: 047 | 0.000223 | 3.6518 | 74 | 300 | 13 | 8258 |
| KEGG | Leukocyte transendothelial | KEGG: 046 | 0.000234 | 3.629985 | 111 | 300 | 16 | 8258 |
| | migration | | | | | | | |
| KEGG | Aldosterone synthesis and secretion | KEGG: 049 | 0.000258 | 3.588452 | 99 | 300 | 15 | 8258 |
| KEGG | NOD-like receptor signaling | KEGG: 046 | 0.00026 | 3.585625 | 197 | 300 | 22 | 8258 |
| | pathway | | | | | | | |
| KEGG | Huntington disease | KEGG: 050 | 0.000312 | 3.5062 | 184 | 300 | 21 | 8258 |
| KEGG | Th17 cell differentiation | KEGG: 0461 | 0.000334 | 3.476564 | 101 | 300 | 15 | 8258 |
| KEGG | Measles | KEGG: 051 | 0.000364 | 3.438643 | 142 | 300 | 18 | 8258 |
| KEGG | Autophagy - animal | KEGG: 041 | 0.00055 | 3.259982 | 132 | 300 | 17 | 8258 |
| KEGG | Prolactin signaling pathway | KEGG: 049 | 0.000721 | 3.14232 | 70 | 300 | 12 | 8258 |
| KEGG | Influenza A | KEGG: 051 | 0.000749 | 3.125542 | 164 | 300 | 19 | 8258 |
| KEGG | Adipocytokine signaling pathway | KEGG: 049 | 0.000839 | 3.07605 | 71 | 300 | 12 | 8258 |
| KEGG | Rheumatoid arthritis | KEGG: 053 | 0.00084 | 3.075848 | 83 | 300 | 13 | 8258 |
| KEGG | Epstein-Barr virus infection | KEGG: 051 | 0.001265 | 2.89803 | 217 | 300 | 22 | 8258 |
| KEGG | Cellular senescence | KEGG: 042 | 0.001266 | 2.89768 | 170 | 300 | 19 | 8258 |
| KEGG | Apoptosis - multiple species | KEGG: 042 | 0.001434 | 2.843344 | 32 | 300 | 8 | 8258 |
| KEGG | Mitophagy - animal | KEGG: 041 | 0.001459 | 2.836081 | 63 | 300 | 11 | 8258 |
| KEGG | Necroptosis | KEGG: 042 | 0.001629 | 2.788113 | 173 | 300 | 19 | 8258 |
| KEGG | B cell receptor signaling pathway | KEGG: 046 | 0.001982 | 2.702873 | 77 | 300 | 12 | 8258 |
| KEGG | Neuroactive ligand-receptor | KEGG: 040 | 0.002458 | 2.609344 | 348 | 300 | 29 | 8258 |
| | interaction | | | | | | | |
| KEGG | Tight junction | KEGG: 045 | 0.003252 | 2.4879 | 166 | 300 | 18 | 8258 |
| KEGG | Lysosome | KEGG: 041 | 0.003468 | 2.459873 | 122 | 300 | 15 | 8258 |
| KEGG | Cortisol synthesis and secretion | KEGG: 049 | 0.003557 | 2.448888 | 69 | 300 | 11 | 8258 |
| KEGG | Insulin secretion | KEGG: 049 | 0.004298 | 2.366705 | 83 | 300 | 12 | 8258 |
| KEGG | Th1 and T112 cell differentiation | KEGG: 0461 | 0.006156 | 2.210707 | 86 | 300 | 12 | 8258 |
| KEGG | Viral protein interaction with | KEGG: 040 | 0.006484 | 2.188182 | 100 | 300 | 13 | 8258 |
| | cytokine and cytokine receptor | | | | | | | |
| KEGG | Platinum drug resistance | KEGG: 015 | 0.006935 | 2.158927 | 74 | 300 | 11 | 8258 |
| KEGG | mTOR signaling pathway | KEGG: 041 | 0.008584 | 2.066301 | 147 | 300 | 16 | 8258 |
| KEGG | Amoebiasis | KEGG: 051 | 0.008852 | 2.052977 | 103 | 300 | 13 | 8258 |
| KEGG | Hematopoietic cell lineage | KEGG: 046 | 0.014831 | 1.828833 | 94 | 300 | 12 | 8258 |
| KEGG | Natural killer cell mediated | KEGG: 0461 | 0.017459 | 1.757978 | 110 | 300 | 13 | 8258 |
| | cytotoxicity | | | | | | | |
| KEGG | Sphingolipid metabolism | KEGG: 006 | 0.026682 | 1.573776 | 47 | 300 | 8 | 8258 |
| KEGG | IL-17 signaling pathway | KEGG: 0461 | 0.033802 | 1.471062 | 88 | 300 | 11 | 8258 |

TABLE 18
From TTPB_0400_final document, Differentially Expressed Genes - GProfiler Results - DIV14

| | | Term | Adjusted | Negative | Term | Query | Intersection | Effective |
|---|---|---|---|---|---|---|---|---|
| Source | Term Name | Id | P Value | Log | Size | Size | Size | DOM |
| KEGG | Glutamatergic synapse | KEGG: 047 | 6.83E−25 | 24.16587 | 112 | 288 | 37 | 8258 |
| KEGG | Long-term potentiation | KEGG: 047 | 1.24E−21 | 20.90584 | 62 | 288 | 27 | 8258 |
| KEGG | Dopaminergic synapse | KEGG: 047 | 2.26E−18 | 17.64579 | 127 | 288 | 33 | 8258 |
| KEGG | Human cytomegalovirus infection | KEGG: 05H | 3.23E−18 | 17.49108 | 242 | 288 | 44 | 8258 |
| KEGG | Retrograde endocannabinoid | KEGG: 047 | 1.21E−16 | 15.91758 | 143 | 288 | 33 | 8258 |
| | signaling | | | | | | | |
| KEGG | Amphetamine addiction | KEGG: 050 | 5.10E−16 | 15.29258 | 64 | 288 | 23 | 8258 |
| KEGG | Nicotine addiction | KEGG: 050 | 7.66E−16 | 15.11599 | 40 | 288 | 19 | 8258 |
| KEGG | Amyotrophic lateral sclerosis (ALS) | KEGG: 050 | 8.70E−16 | 15.06072 | 52 | 288 | 21 | 8258 |
| KEGG | MAPK signaling pathway | KEGG: 040 | 2.63E−15 | 14.58087 | 286 | 288 | 44 | 8258 |
| KEGG | Oxytocin signaling pathway | KEGG: 049 | 2.65E−15 | 14.57725 | 147 | 288 | 32 | 8258 |
| KEGG | Calcium signaling pathway | KEGG: 040 | 2.87E−15 | 14.54209 | 190 | 288 | 36 | 8258 |
| KEGG | Long-term depression | KEGG: 047 | 1.89E−14 | 13.72454 | 59 | 288 | 21 | 8258 |
| KEGG | Circadian entrainment | KEGG: 047 | 1.06E−13 | 12.97527 | 96 | 288 | 25 | 8258 |
| KEGG | GABAergic synapse | KEGG: 047 | 1.42E−12 | 11.84708 | 88 | 288 | 23 | 8258 |
| KEGG | GnRH signaling pathway | KEGG: 049 | 1.42E−12 | 11.84708 | 88 | 288 | 23 | 8258 |
| KEGG | Serotonergic synapse | KEGG: 047 | 3.19E−12 | 11.49635 | 130 | 288 | 27 | 8258 |
| KEGG | Cholinergic synapse | KEGG: 047 | 3.30E−12 | 11.48153 | 110 | 288 | 25 | 8258 |
| KEGG | AGE-RAGE signaling pathway in | KEGG: 049 | 2.21E−11 | 10.65472 | 99 | 288 | 23 | 8258 |
| | diabetic complications | | | | | | | |
| KEGG | Endocrine resistance | KEGG: 015 | 3.13E−11 | 10.50494 | 91 | 288 | 22 | 8258 |
| KEGG | Apelin signaling pathway | KEGG: 043 | 5.29E−11 | 10.27644 | 134 | 288 | 26 | 8258 |
| KEGG | cAMP signaling pathway | KEGG: 040 | 9.42E−11 | 10.02603 | 209 | 288 | 32 | 8258 |
| KEGG | Sphingolipid signaling pathway | KEGG: 040 | 1.34E−09 | 8.872817 | 119 | 288 | 23 | 8258 |
| KEGG | Alzheimer disease | KEGG: 050 | 1.48E−09 | 8.829586 | 166 | 288 | 27 | 8258 |
| KEGG | HIF-1 signaling pathway | KEGG: 040 | 1.54E−09 | 8.813155 | 109 | 288 | 22 | 8258 |
| KEGG | Human T-cell leukemia virus 1 | KEGG: 051 | 2.13E−09 | 8.672017 | 234 | 288 | 32 | 8258 |
| | infection | | | | | | | |
| KEGG | Rap1 signaling pathway | KEGG: 040 | 2.73E−09 | 8.563207 | 209 | 288 | 30 | 8258 |
| KEGG | Human papillomavirus infection | KEGG: 051 | 5.20E−09 | 8.284028 | 346 | 288 | 39 | 8258 |
| KEGG | Thyroid hormone signaling pathway | KEGG: 049 | 5.62E−09 | 8.250451 | 116 | 288 | 22 | 8258 |
| KEGG | Human immunodeficiency virus 1 | KEGG: 051 | 1.60E−08 | 7.797198 | 224 | 288 | 30 | 8258 |
| | infection | | | | | | | |
| KEGG | Apoptosis | KEGG: 042 | 1.96E−08 | 7.70716 | 135 | 288 | 23 | 8258 |
| KEGG | VEGF signaling pathway | KEGG: 043 | 4.28E−08 | 7.368253 | 56 | 288 | 15 | 8258 |
| KEGG | Cocaine addiction | KEGG: 050 | 4.77E−08 | 7.321076 | 48 | 288 | 14 | 8258 |
| KEGG | C-type lectin receptor signaling | KEGG: 046 | 8.02E−08 | 7.095622 | 109 | 288 | 20 | 8258 |
| | pathway | | | | | | | |
| KEGG | TNF signaling pathway | KEGG: 046 | 8.02E−08 | 7.095622 | 109 | 288 | 20 | 8258 |
| KEGG | Ras signaling pathway | KEGG: 040 | 1.14E−07 | 6.944369 | 228 | 288 | 29 | 8258 |
| KEGG | Relaxin signaling pathway | KEGG: 049 | 2.19E−07 | 6.659409 | 127 | 288 | 21 | 8258 |
| KEGG | Synaptic vesicle cycle | KEGG: 047 | 3.43E−07 | 6.464615 | 74 | 288 | 16 | 8258 |
| KEGG | Gap junction | KEGG: 045 | 3.92E−07 | 6.406854 | 85 | 288 | 17 | 8258 |
| KEGG | EGFR tyrosine kinase inhibitor | KEGG: 015 | 5.19E−07 | 6.285135 | 76 | 288 | 16 | 8258 |
| | resistance | | | | | | | |
| KEGG | Hepatitis B | KEGG: 051 | 5.44E−07 | 6.264136 | 159 | 288 | 23 | 8258 |
| KEGG | Morphine addiction | KEGG: 050 | 6.84E−07 | 6.164672 | 88 | 288 | 17 | 8258 |
| KEGG | Chemokine signaling pathway | KEGG: 040 | 1.05E−06 | 5.979275 | 192 | 288 | 25 | 8258 |
| KEGG | FoxO signaling pathway | KEGG: 040 | 1.11E−06 | 5.954677 | 126 | 288 | 20 | 8258 |
| KEGG | Axon guidance | KEGG: 043 | 1.17E−06 | 5.932433 | 179 | 288 | 24 | 8258 |
| KEGG | ErbB signaling pathway | KEGG: 040 | 1.37E−06 | 5.861974 | 81 | 288 | 16 | 8258 |
| KEGG | Melanogenesis | KEGG: 049 | 2.7E−06 | 5.568207 | 96 | 288 | 17 | 8258 |
| KEGG | Hepatitis C | KEGG: 051 | 2.73E−06 | 5.564504 | 159 | 288 | 22 | 8258 |
| KEGG | Wnt signaling pathway | KEGG: 043 | 3.06E−06 | 5.513972 | 160 | 288 | 22 | 8258 |
| KEGG | Inflammatory mediator regulation of | KEGG: 047 | 3.59E−06 | 5.444375 | 122 | 288 | 19 | 8258 |
| | TRP channels | | | | | | | |
| KEGG | T cell receptor signaling pathway | KEGG: 046 | 5.08E−06 | 5.29385 | 100 | 288 | 17 | 8258 |
| KEGG | Longevity regulating pathway - | KEGG: 042 | 1.02E−05 | 4.990548 | 60 | 288 | 13 | 8258 |
| | multiple species | | | | | | | |
| KEGG | PI3K-Akt signaling pathway | KEGG: 041 | 1.51E−05 | 4.822032 | 350 | 288 | 33 | 8258 |
| KEGG | Renin secretion | KEGG: 049 | 1.99E−05 | 4.700529 | 74 | 288 | 14 | 8258 |
| KEGG | Aldosterone synthesis and secretion | KEGG: 049 | 2.62E−05 | 4.581372 | 99 | 288 | 16 | 8258 |
| KEGG | Fluid shear stress and atherosclerosis | KEGG: 054 | 2.7E−05 | 4.568806 | 138 | 288 | 19 | 8258 |
| KEGG | Longevity regulating pathway | KEGG: 042 | 3.05E−05 | 4.515381 | 88 | 288 | 15 | 8258 |
| KEGG | Neuroactive ligand-receptor | KEGG: 040 | 4.17E−05 | 4.379415 | 348 | 288 | 32 | 8258 |
| | interaction | | | | | | | |
| KEGG | Glucagon signaling pathway | KEGG: 049 | 4.59E−05 | 4.338066 | 103 | 288 | 16 | 8258 |
| KEGG | Insulin resistance | KEGG: 049 | 5.26E−05 | 4.279199 | 104 | 288 | 16 | 8258 |
| KEGG | GnRH secretion | KEGG: 049 | 5.46E−05 | 4.262597 | 58 | 288 | 12 | 8258 |
| KEGG | Neurotrophin signaling pathway | KEGG: 047 | 6.05E−05 | 4.217991 | 118 | 288 | 17 | 8258 |
| KEGG | Endocrine and other factor-regulated | KEGG: 049 | 6.65E−05 | 4.17741 | 59 | 288 | 12 | 8258 |
| | calcium reabsorption | | | | | | | |
| KEGG | Tuberculosis | KEGG: 051 | 6.66E−05 | 4.176277 | 175 | 288 | 21 | 8258 |
| KEGG | Insulin secretion | KEGG: 049 | 8.71E−05 | 4.059881 | 83 | 288 | 14 | 8258 |
| KEGG | Focal adhesion | KEGG: 045 | 9.56E−05 | 4.019462 | 194 | 288 | 22 | 8258 |
| KEGG | Viral protein interaction with | KEGG: 040 | 0.000167 | 3.777115 | 100 | 288 | 15 | 8258 |
| | cytokine and cytokine receptor | | | | | | | |
| KEGG | Chagas disease (American | KEGG: 051 | 0.00019 | 3.720949 | 101 | 288 | 15 | 8258 |
| | trypanosomiasis) | | | | | | | |
| KEGG | Amoebiasis | KEGG: 051 | 0.000245 | 3.610826 | 103 | 288 | 15 | 8258 |
| KEGG | Parathyroid hormone synthesis, | KEGG: 049 | 0.000277 | 3.556837 | 104 | 288 | 15 | 8258 |
| | secretion and action | | | | | | | |
| KEGG | Type II diabetes mellitus | KEGG: 049 | 0.000375 | 3.425435 | 47 | 288 | 10 | 8258 |
| KEGG | Influenza A | KEGG: 051 | 0.000391 | 3.407917 | 164 | 288 | 19 | 8258 |
| KEGG | Toxoplasmosis | KEGG: 051 | 0.000399 | 3.398988 | 107 | 288 | 15 | 8258 |
| KEGG | cGMP-PKG signaling pathway | KEGG: 040 | 0.000512 | 3.290668 | 167 | 288 | 19 | 8258 |
| KEGG | Huntington disease | KEGG: 050 | 0.000582 | 3.234858 | 184 | 288 | 20 | 8258 |
| KEGG | Cushing syndrome | KEGG: 049 | 0.000747 | 3.126436 | 156 | 288 | 18 | 8258 |
| KEGG | Measles | KEGG: 051 | 0.000818 | 3.087062 | 142 | 288 | 17 | 8258 |
| KEGG | Mitophagy - animal | KEGG: 041 | 0.000938 | 3.027832 | 63 | 288 | 11 | 8258 |
| KEGG | Pertussis | KEGG: 051 | 0.000941 | 3.026232 | 75 | 288 | 12 | 8258 |
| KEGG | Adrenergic signaling in | KEGG: 042 | 0.000989 | 3.004906 | 144 | 288 | 17 | 8258 |
| | cardiomyocytes | | | | | | | |
| KEGG | Apoptosis - multiple species | KEGG: 042 | 0.001006 | 2.997218 | 32 | 288 | 8 | 8258 |
| KEGG | Salivary secretion | KEGG: 049 | 0.001084 | 2.965135 | 76 | 288 | 12 | 8258 |
| KEGG | Phospholipase D signaling pathway | KEGG: 040 | 0.00119 | 2.924349 | 146 | 288 | 17 | 8258 |
| KEGG | Autophagy - animal | KEGG: 041 | 0.001292 | 2.888616 | 132 | 288 | 16 | 8258 |
| KEGG | Platelet activation | KEGG: 046 | 0.001502 | 2.823229 | 119 | 288 | 15 | 8258 |
| KEGG | Epstein-Barr virus infection | KEGG: 051 | 0.00208 | 2.682018 | 217 | 288 | 21 | 8258 |
| KEGG | Cellular senescence | KEGG: 042 | 0.002463 | 2.608511 | 170 | 288 | 18 | 8258 |
| KEGG | Natural killer cell mediated | KEGG: 046 | 0.002595 | 2.585908 | 110 | 288 | 14 | 8258 |
| | cytotoxicity | | | | | | | |
| KEGG | Rheumatoid arthritis | KEGG: 053 | 0.00272 | 2.565405 | 83 | 288 | 12 | 8258 |
| KEGG | Sphingolipid metabolism | KEGG: 006 | 0.002858 | 2.54387 | 47 | 288 | 9 | 8258 |
| KEGG | Adipocytokine signaling pathway | KEGG: 049 | 0.003043 | 2.516718 | 71 | 288 | 11 | 8258 |
| KEGG | Cell adhesion molecules (CAMs) | KEGG: 045 | 0.003658 | 2.436728 | 159 | 288 | 17 | 8258 |
| KEGG | B cell receptor signaling pathway | KEGG: 046 | 0.006593 | 2.180898 | 77 | 288 | 11 | 8258 |
| KEGG | Leukocyte transendothelial | KEGG: 046 | 0.012052 | 1.918932 | 111 | 288 | 13 | 8258 |
| | migration | | | | | | | |
| KEGG | Prion diseases | KEGG: 050 | 0.013955 | 1.855273 | 34 | 288 | 7 | 8258 |
| KEGG | Prolactin signaling pathway | KEGG: 049 | 0.014153 | 1.849162 | 70 | 288 | 10 | 8258 |
| KEGG | p53 signaling pathway | KEGG: 041 | 0.015974 | 1.796588 | 71 | 288 | 10 | 8258 |
| KEGG | African trypanosomiasis | KEGG: 051 | 0.01693 | 1.771351 | 35 | 288 | 7 | 8258 |
| KEGG | mTOR signaling pathway | KEGG: 041 | 0.017812 | 1.749285 | 147 | 288 | 15 | 8258 |
| KEGG | Thyroid hormone synthesis | KEGG: 049 | 0.017989 | 1.744999 | 72 | 288 | 10 | 8258 |
| KEGG | Th17 cell differentiation | KEGG: 046 | 0.019131 | 1.71826 | 101 | 288 | 12 | 8258 |
| KEGG | Yersinia infection | KEGG: 051 | 0.024426 | 1.612149 | 119 | 288 | 13 | 8258 |
| KEGG | Necroptosis | KEGG: 042 | 0.034049 | 1.467892 | 173 | 288 | 16 | 8258 |
| KEGG | Fc epsilon RI signaling pathway | KEGG: 046 | 0.038692 | 1.412373 | 65 | 288 | 9 | 8258 |
| KEGG | Ferroptosis | KEGG: 042 | 0.047318 | 1.324974 | 41 | 288 | 7 | 8258 |

### REFERENCES CITED

Durrenberger P F, Fernando F S, Kashefi S N, Bonnert T P et al. Common mechanisms in neurodegeneration and neuroinflammation: a BrainNet Europe gene expression microarray study. J Neural Transm 2015 July; 122(7): 1055-68. PMID: 25119539
Hodges A, Strand A D, Aragaki A K, Kuhn A et al. Regional and cellular gene expression changes in human Huntington's disease brain. Hum Mol Genet 2006 Mar. 15; 15(6):965-77. PMID: 16467349

1. A cultured whole brain slice obtained from an adult rodent of at least 17 days of age, wherein the brain slice comprises adult microglia and has been cultured for at least 3 days in vitro.

2. A cultured whole brain slice obtained from an adult rodent, wherein the cultured whole brain slice has been cultured in a serum-free or reduced serum culture medium, and wherein the cultured whole brain slice has maintained viability in the serum-free or reduced serum culture medium for one day or more.

3. The cultured whole brain slice of claim 2, wherein, prior to culturing in the serum-free or reduced serum culture medium, the cultured whole brain slice was cultured in a recovery cell culture medium.

4. The cultured whole brain slice of claim 3, wherein the recovery cell culture medium comprises serum, optionally more than about 5% serum, optionally at least about 10% serum, optionally about 20% serum, optionally wherein the serum is bovine, equine, ovine, caprine or porcine, optionally wherein the serum is heat-inactivated.

5. The cultured whole brain slice of any one of claims 2-4, wherein the cultured whole brain slice was cultured in the recovery cell culture medium for more than 2 days, optionally for at least 3 days, optionally for at least 4 days.

6. The cultured whole brain slice of any one of claims 2-5, wherein the serum-free or reduced serum culture medium comprises no more than about 5% serum, optionally wherein the serum-free or reduced serum culture medium is a serum-free culture medium.

7. The cultured whole brain slice of any one of claims 4-6, wherein the serum-free or reduced serum culture medium comprises no more than half the serum concentration present in the recovery cell culture medium.

8. A cultured whole brain slice, wherein the cultured whole brain slice exhibits at least one characteristic of chronic neuroinflammation.

9. The cultured whole brain slice of claim 8, wherein at least one characteristic of chronic inflammation comprises increased release of one or more pro-inflammatory cytokines as compared to naïve rodent cerebrospinal fluid, optionally wherein the cultured whole brain slice exhibits the increased release at DIV1, optionally wherein the cultured whole brain slice exhibits the increased release at DIV5, DIV6, DIV7, or greater than DIV7, optionally wherein the one or more pro-inflammatory cytokines are selected from IL-6, TNF-α, IL-1, and IFNγ.

10. The cultured whole brain slice of claim 8 or 9, wherein the at least one characteristic of chronic inflammation comprises a gene expression signature characteristic of chronic neuroinflammation.

11. The cultured whole brain slice of claim 9, wherein the gene expression signature comprises increases in one or more genes selected from the group consisting of: C3, Gfap, Cd9, Msn, Cd68, Klk6, Gjb1, Trem2, Gusb, Trf, Bcas1, Tgfb1, Ccnd1, Grn, Olig2, Tnfrsf1a, Fgf2, Sgpl1, C1qc, Mbp, S100b, C1qa, Myrf, Csf1, Cxcl16, Cd14, Nes, Gsn, Lrrc25, Mag, Ugt8a, Car2, Tspo, Sox10, Cybb, Spi1, Stat3, Pmp22, Serpinb6a, and Arhgef10, In certain embodiments, and, optionally, decreases in one or more genes selected from the group consisting of: in Gabra1, Syt1, Gabrb2, Slc12a5, Pcsk2, Calb1, Grin1, Rasgrp1, Scn1a, Grin2b, Cx3cl1, Drd1, Gabrg2, Scn2a1, Vip, Gad1, Camk4, Prkcg, Syt13, Thy1, Cntnap1, Cnksr2, Ryr2, Cnr1, Sncb, Unc13a, Nell2, Cdk5r1, Cacna1b, Epha7, Ptprn2, Grin2a, Gabra4, Cds1, Fgf14, Grm1, Ipcef1, Htr1a, Slc8a1, Gad2.

12. A cultured whole brain slice, wherein the cultured whole brain slice exhibits a gene expression signature, wherein the gene expression signature comprises stable correlation with a human neuroinflammatory disease signature of interest.

13. The cultured whole brain slice of claim 12, wherein the human neuroinflammatory disease signature of interest is selected from the group consisting of AD neuroinflammatory subset, Huntington's disease (HD), ALS neuroinflammatory subset, progressive supranuclear palsy (PSP), and frontotemporal dementia (FTD).

14. The cultured whole brain slice of any one of the preceding claims, wherein the cultured whole brain slice is obtained from a rodent.

15. The cultured whole brain slice of any one of the preceding claims, wherein the rodent is at least 17 days of age.

16. The cultured whole brain slice of claim 15, wherein the adult rodent is at least 19 days of age.

17. The cultured whole brain slice of claim 15, wherein the adult rodent is at least 21 days of age, optionally at least 42 days of age.

18. The method of any one of claims 14-17, wherein the rodent is a mouse or rat.

19. The cultured whole brain slice of any one of the preceding claims, comprising cortical brain tissue.

20. The cultured whole brain slice of claim 18, further comprising subcortical brain tissue.

21. The cultured whole brain slice of claim 20, wherein the cortical and subcortical brain tissue comprises neurons or their progenitor cells.

22. The cultured whole brain slice of claim 20, wherein the cortical and subcortical brain tissue comprises microglia or their progenitor cells.

23. The cultured whole brain slice of claim 20, wherein the cortical and subcortical brain tissue comprises astrocytes.

24. The cultured whole brain slice of claim 20, wherein the cortical and subcortical brain tissue comprises oligodendrocytes or their progenitor cells.

25. The cultured whole brain slice of claim 20, wherein the cortical and subcortical brain tissue comprises endothelial cells.

26. The cultured whole brain slice of any one of the preceding claims, comprising a thickness of about 100-400 μm, optionally about 300 μm.

27. An assay system, comprising the cultured whole brain slice of any one of the preceding claims and a cell culture medium.

28. The assay system of claim 27, wherein the cell culture medium is a serum-free or reduced serum culture medium.

29. The assay system of any one of the preceding claims, comprising a plurality of cultured whole brain slices according to any one of the preceding claims.

30. The assay system of any one of the preceding claims, further comprising a candidate therapeutic.

31. The assay system of claim 30, wherein the candidate therapeutic is administered to the cultured whole brain slice.

32. A method of producing a cultured whole brain slice, comprising:
a. culturing a whole brain slice obtained from a rodent in a recovery cell culture medium comprising serum; and
b. culturing the whole brain slice in a serum-free or reduced serum culture medium.

33. The method of claim 32, wherein the recovery cell culture medium comprises more than about 5% serum, optionally at least about 10% serum, optionally about 20% serum, optionally wherein the serum is bovine, equine, ovine, caprine or porcine, optionally wherein the serum is heat-inactivated.

34. The method of claim 32 or 33, comprising culturing the cultured whole brain slice in the recovery cell culture medium for more than 2 days, optionally for at least 3 days, optionally for at least 4 days.

35. The method of any one of claims 32-34, comprising culturing the brain slice in the serum-free or reduced serum culture medium for at least 1 day, optionally for at least 2, 3, 4, 5, or more than 5 days.

36. The method of any one of claims 32-35, wherein the serum-free or reduced serum culture medium comprises no more than about 5% serum, optionally wherein the serum-free or reduced serum culture medium is a serum-free culture medium.

37. The method of any one of claims 32-36, wherein the serum-free or reduced serum culture medium comprises no more than half the serum concentration present in the recovery cell culture medium.

38. The method of any one of claims 32-36, wherein the whole brain slice is obtained from an adult rodent at least 17 days of age, optionally at least 19 days of age, optionally at least 21 days of age, optionally at least 42 days of age.

39. The method of any one of claims 32-38, wherein the whole brain slice comprises cortical and subcortical brain tissue.

40. The method of claim 39, wherein the cortical and subcortical brain tissue comprises neurons or their progenitors.

41. The method of claim 39, wherein the cortical and subcortical brain tissue comprises microglia or their progenitors.

42. The method of claim 39, wherein the cortical and subcortical brain tissue comprises astrocytes.

43. The method of claim 39, wherein the cortical and subcortical brain tissue comprises oligodendrocytes or their precursors.
